SEQUENCE LISTING (33 sequences)

SEQ ID NO: 1 - protein, 2347 aa, Artificial Sequence
  Human ROS
MKNIYCLIPKLVNFATLGCLWISVVQCTVLNSCLKSCVTNLGQQLDLGTPHNLSEPCIQGCHFWNSVDQKNCALKCRESCEVGCSSAEGAYEEEVLENADLPTAPFASSIGSHNMTLRWKSANFSGVKYIIQWKYAQLLGSWTYTKTVSRPSYVVKPLHPFTEYIFRVVWIFTAQLQLYSPPSPSYRTHPHGVPETAPLIRNIESSSPDTVEVSWDPPQFPGGPILGYNLRLISKNQKLDAGTQRTSFQFYSTLPNTIYRFSIAAVNEVGEGPEAESSITTSSSAVQQEEQWLFLSRKTSLRKRSLKHLVDEAHCLRLDAIYHNITGISVDVHQQIVYFSEGTLIWAKKAANMSDVSDLRIFYRGSGLISSISIDWLYQRMYFIMDELVCVCDLENCSNIEEITPPSISAPQKIVADSYNGYVFYLLRDGIYRADLPVPSGRCAEAVRIVESCTLKDFAIKPQAKRIIYFNDTAQVFMSTFLDGSASHLILPRIPFADVKSFACENNDFLVTDGKVIFQQDALSFNEFIVGCDLSHIEEFGFGNLVIFGSSSQLHPLPGRPQELSVLFGSHQALVQWKPPALAIGANVILISDIIELFELGPSAWQNWTYEVKVSTQDPPEVTHIFLNISGTMLNVPELQSAMKYKVSVRASSPKRPGPWSEPSVGTTLVPASEPPFIMAVKEDGLWSKPLNSFGPGEFLSSDIGNVSDMDWYNNSLYYSDTKGDVFVWLLNGTDISENYHLPSIAGAGALAFEWLGHFLYWAGKTYVIQRQSVLTGHTDIVTHVKLLVNDMVVDSVGGYLYWTTLYSVESTRLNGESSLVLQTQPWFSGKKVIALTLDLSDGLLYWLVQDSQCIHLYTAVLRGQSTGDTTITEFAAWSTSEISQNALMYYSGRLFWINGFRIITTQEIGQKTSVSVLEPARFNQFTIIQTSLKPLPGNFSFTPKVIPDSVQESSFRIEGNASSFQILWNGPPAVDWGVVFYSVEFSAHSKFLASEQHSLPVFTVEGLEPYALFNLSVTPYTYWGKGPKTSLSLRAPETVPSAPENPRIFILPSGKCCNKNEVVVEFRWNKPKHENGVLTKFEIFYNISNQSITNKTCEDWIAVNVTPSVMSFQLEGMSPRCFIAFQVRAFTSKGPGPYADVVKSTTSEINPFPHLITLLGNKIVFLDMDQNQVVWTFSAERVISAVCYTADNEMGYYAEGDSLFLLHLHNRSSSELFQDSLVFDITVITIDWISRHLYFALKESQNGMQVFDVDLEHKVKYPREVKIHNRNSTIISFSVYPLLSRLYWTEVSNFGYQMFYYSIISHTLHRILQPTATNQQNKRNQCSCNVTEFELSGAMAIDTSNLEKPLIYFAKAQEIWAMDLEGCQCWRVITVPAMLAGKTLVSLTVDGDLIYWIITAKDSTQIYQAKKGNGAIVSQVKALRSRHILAYSSVMQPFPDKAFLSLASDTVEPTILNATNTSLTIRLPLAKTNLTWYGITSPTPTYLVYYAEVNDRKNSSDLKYRILEFQDSIALIEDLQPFSTYMIQIAVKNYYSDPLEHLPPGKEIWGKTKNGVPEAVQLINTTVRSDTSLIISWRESHKPNGPKESVRYQLAISHLALIPETPLRQSEFPNGRLTLLVTRLSGGNIYVLKVLACHSEEMWCTESHPVTVEMFNTPEKPYSLVPENTSLQFNWKAPLNVNLIRFWVELQKWKYNEFYHVKTSCSQGPAYVCNITNLQPYTSYNVRVVVVYKTGENSTSLPESFKTKAGVPNKPGIPKLLEGSKNSIQWEKAEDNGCRITYYILEIRKSTSNNLQNQNLRWKMTFNGSCSSVCTWKSKNLKGIFQFRVVAANNLGFGEYSGISENIILVGDDFWIPETSFILTIIVGIFLVVTIPLTFVWHRRLKNQKSAKEGVTVLINEDKELAELRGLAAGVGLANACYAIHTLPTQEEIENLPAFPREKLTLRLLLGSGAFGEVYEGTAVDILGVGSGEIKVAVKTLKKGSTDQEKIEFLKEAHLMSKFNHPNILKQLGVCLLNEPQYIILELMEGGDLLTYLRKARMATFYGPLLTLVDLVDLCVDISKGCVYLERMHFIHRDLAARNCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQDQLQLFRNFFLNSIYKSRDEANNSGVINESFEGEDGDVICLNSDDIMPVALMETKNREGLNYMVLATECGQGEEKSEGPLGSQESESCGLRKEEKEPHADKDFCQEKQVAYCPSGKPEGLNYACLTHSGYGDGSD

SEQ ID NO: 2 - DNA, 7368 bp, Artificial Sequence
  Human ROS
caagctttcaagcattcaaaggtctaaatgaaaaaggctaagtattatttcaaaaggcaagtatatcctaatatagcaaaacaaacaaagcaaaatccatcagctactcctccaattgaagtgatgaagcccaaataattcatatagcaaaatggagaaaattagaccggccatctaaaaatctgccattggtgaagtgatgaagaacatttactgtcttattccgaagcttgtcaattttgcaactcttggctgcctatggatttctgtggtgcagtgtacagttttaaatagctgcctaaagtcgtgtgtaactaatctgggccagcagcttgaccttggcacaccacataatctgagtgaaccgtgtatccaaggatgtcacttttggaactctgtagatcagaaaaactgtgctttaaagtgtcgggagtcgtgtgaggttggctgtagcagcgcggaaggtgcatatgaagaggaagtactggaaaatgcagacctaccaactgctccctttgcttcttccattggaagccacaatatgacattacgatggaaatctgcaaacttctctggagtaaaatacatcattcagtggaaatatgcacaacttctgggaagctggacttatactaagactgtgtccagaccgtcctatgtggtcaagcccctgcaccccttcactgagtacattttccgagtggtttggatcttcacagcgcagctgcagctctactcccctccaagtcccagttacaggactcatcctcatggagttcctgaaactgcacctttgattaggaatattgagagctcaagtcccgacactgtggaagtcagctgggatccacctcaattcccaggtggacctattttgggttataacttaaggctgatcagcaaaaatcaaaaattagatgcagggacacagagaaccagtttccagttttactccactttaccaaatactatctacaggttttctattgcagcagtaaatgaagttggtgagggtccagaagcagaatctagtattaccacttcatcttcagcagttcaacaagaggaacagtggctctttttatccagaaaaacttctctaagaaagagatctttaaaacatttagtagatgaagcacattgccttcggttggatgctatataccataatattacaggaatatctgttgatgtccaccagcaaattgtttatttctctgaaggaactctcatatgggcgaagaaggctgccaacatgtctgatgtatctgacctgagaattttttacagaggttcaggattaatttcttctatctccatagattggctttatcaaagaatgtatttcatcatggatgaactggtatgtgtctgtgatttagagaactgctcaaacatcgaggaaattactccaccctctattagtgcacctcaaaaaattgtggctgattcatacaatgggtatgtcttttacctcctgagagatggcatttatagagcagaccttcctgtaccatctggccggtgtgcagaagctgtgcgtattgtggagagttgcacgttaaaggactttgcaatcaagccacaagccaagcgaatcatttacttcaatgacactgcccaagtcttcatgtcaacatttctggatggctctgcttcccatctcatcctacctcgcatcccctttgctgatgtgaaaagttttgcttgtgaaaacaatgactttcttgtcacagatggcaaggtcattttccaacaggatgctttgtcttttaatgaattcatcgtgggatgtgacctgagtcacatagaagaatttgggtttggtaacttggtcatctttggctcatcctcccagctgcaccctctgccaggccgcccgcaggagctttcggtgctgtttggctctcaccaggctcttgttcaatggaagcctcctgcccttgccataggagccaatgtcatcctgatcagtgatattattgaactctttgaattaggcccttctgcctggcagaactggacctatgaggtgaaagtatccacccaagaccctcctgaagtcactcatattttcttgaacataagtggaaccatgctgaatgtacctgagctgcagagtgctatgaaatacaaggtttctgtgagagcaagttctccaaagaggccaggcccctggtcagagccctcagtgggtactaccctggtgccagctagtgaaccaccatttatcatggctgtgaaagaagatgggctttggagtaaaccattaaatagctttggcccaggagagttcttatcctctgatataggaaatgtgtcagacatggattggtataacaacagcctctactacagtgacacgaaaggcgacgtttttgtgtggctgctgaatgggacggatatctcagagaattatcacctacccagcattgcaggagcaggggctttagcttttgagtggctgggtcactttctctactgggctggaaagacatatgtgatacaaaggcagtctgtgttgacgggacacacagacattgttacccacgtgaagctattggtgaatgacatggtggtggattcagttggtggatatctctactggaccacactctattcagtggaaagcaccagactaaatggggaaagttcccttgtactacagacacagccttggttttctgggaaaaaggtaattgctctaactttagacctcagtgatgggctcctgtattggttggttcaagacagtcaatgtattcacctgtacacagctgttcttcggggacagagcactggggataccaccatcacagaatttgcagcctggagtacttctgaaatttcccagaatgcactgatgtactatagtggtcggctgttctggatcaatggctttaggattatcacaactcaagaaataggtcagaaaaccagtgtctctgttttggaaccagccagatttaatcagttcacaattattcagacatcccttaagcccctgccagggaacttttcctttacccctaaggttattccagattctgttcaagagtcttcatttaggattgaaggaaatgcttcaagttttcaaatcctgtggaatggtccccctgcggtagactggggtgtagttttctacagtgtagaatttagtgctcattctaagttcttggctagtgaacaacactctttacctgtatttactgtggaaggactggaaccttatgccttatttaatctttctgtcactccttatacctactggggaaagggccccaaaacatctctgtcacttcgagcacctgaaacagttccatcagcaccagagaaccccagaatatttatattaccaagtggaaaatgctgcaacaagaatgaagttgtggtggaatttaggtggaacaaacctaagcatgaaaatggggtgttaacaaaatttgaaattttctacaatatatccaatcaaagtattacaaacaaaacatgtgaagactggattgctgtcaatgtcactccctcagtgatgtcttttcaacttgaaggcatgagtcccagatgctttattgccttccaggttagggcctttacatctaaggggccaggaccatatgctgacgttgtaaagtctacaacatcagaaatcaacccatttcctcacctcataactcttcttggtaacaagatagtttttttagatatggatcaaaatcaagttgtgtggacgttttcagcagaaagagttatcagtgccgtttgctacacagctgataatgagatgggatattatgctgaaggggactcactctttcttctgcacttgcacaatcgctctagctctgagcttttccaagattcactggtttttgatatcacagttattacaattgactggatttcaaggcacctctactttgcactgaaagaatcacaaaatggaatgcaagtatttgatgttgatcttgaacacaaggtgaaatatcccagagaggtgaagattcacaataggaattcaacaataatttctttttctgtatatcctcttttaagtcgcttgtattggacagaagtttccaattttggctaccagatgttctactacagtattatcagtcacaccttgcaccgaattctgcaacccacagctacaaaccaacaaaacaaaaggaatcaatgttcttgtaatgtgactgaatttgagttaagtggagcaatggctattgatacctctaacctagagaaaccattgatatactttgccaaagcacaagagatctgggcaatggatctggaaggctgtcagtgttggagagttatcacagtacctgctatgctcgcaggaaaaacccttgttagcttaactgtggatggagatcttatatactggatcatcacagcaaaggacagcacacagatttatcaggcaaagaaaggaaatggggccatcgtttcccaggtgaaggccctaaggagtaggcatatcttggcttacagttcagttatgcagccttttccagataaagcgtttctgtctctagcttcagacactgtggaaccaactatacttaatgccactaacactagcctcacaatcagattacctctggccaagacaaacctcacatggtatggcatcaccagccctactccaacatacctggtttattatgcagaagttaatgacaggaaaaacagctctgacttgaaatatagaattctggaatttcaggacagtatagctcttattgaagatttacaaccattttcaacatacatgatacagatagctgtaaaaaattattattcagatcctttggaacatttaccaccaggaaaagagatttggggaaaaactaaaaatggagtaccagaggcagtgcagctcattaatacaactgtgcggtcagacaccagcctcattatatcttggagagaatctcacaagccaaatggacctaaagaatcagtccgttatcagttggcaatctcacacctggccctaattcctgaaactcctctaagacaaagtgaatttccaaatggaaggctcactctccttgttactagactgtctggtggaaatatttatgtgttaaaggttcttgcctgccactctgaggaaatgtggtgtacagagagtcatcctgtcactgtggaaatgtttaacacaccagagaaaccttattccttggttccagagaacactagtttgcaatttaattggaaggctccattgaatgttaacctcatcagattttgggttgagctacagaagtggaaatacaatgagttttaccatgttaaaacttcatgcagccaaggtcctgcttatgtctgtaatatcacaaatctacaaccttatacttcatataatgtcagagtagtggtggtttataagacgggagaaaatagcacctcacttccagaaagctttaagacaaaagctggagtcccaaataaaccaggcattcccaaattactagaagggagtaaaaattcaatacagtgggagaaagctgaagataatggatgtagaattacatactatatccttgagataagaaagagcacttcaaataatttacagaaccagaatttaaggtggaagatgacatttaatggatcctgcagtagtgtttgcacatggaagtccaaaaacctgaaaggaatatttcagttcagagtagtagctgcaaataatctagggtttggtgaatatagtggaatcagtgagaatattatattagttggagatgatttttggataccagaaacaagtttcatacttactattatagttggaatatttctggttgttacaatcccactgacctttgtctggcatagaagattaaagaatcaaaaaagtgccaaggaaggggtgacagtgcttataaacgaagacaaagagttggctgagctgcgaggtctggcagccggagtaggcctggctaatgcctgctatgcaatacatactcttccaacccaagaggagattgaaaatcttcctgccttccctcgggaaaaactgactctgcgtctcttgctgggaagtggagcctttggagaagtgtatgaaggaacagcagtggacatcttaggagttggaagtggagaaatcaaagtagcagtgaagactttgaagaagggttccacagaccaggagaagattgaattcctgaaggaggcacatctgatgagcaaatttaatcatcccaacattctgaagcagcttggagtttgtctgctgaatgaaccccaatacattatcctggaactgatggagggaggagaccttcttacttatttgcgtaaagcccggatggcaacgttttatggtcctttactcaccttggttgaccttgtagacctgtgtgtagatatttcaaaaggctgtgtctacttggaacggatgcatttcattcacagggatctggcagctagaaattgccttgtttccgtgaaagactataccagtccacggatagtgaagattggagactttggactcgccagagacatctataaaaatgattactatagaaagagaggggaaggcctgctcccagttcggtggatggctccagaaagtttgatggatggaatcttcactactcaatctgatgtatggtcttttggaattctgatttgggagattttaactcttggtcatcagccttatccagctcattccaaccttgatgtgttaaactatgtgcaaacaggagggagactggagccaccaagaaattgtcctgatgatctgtggaatttaatgacccagtgctgggctcaagaacccgaccaaagacctacttttcatagaattcaggaccaacttcagttattcagaaattttttcttaaatagcatttataagtccagagatgaagcaaacaacagtggagtcataaatgaaagctttgaaggtgaagatggcgatgtgatttgtttgaattcagatgacattatgccagttgctttaatggaaacgaagaaccgagaagggttaaactatatggtacttgctacagaatgtggccaaggtgaagaaaagtctgagggtcctctaggctcccaggaatctgaatcttgtggtctgaggaaagaagagaaggaaccacatgcagacaaagatttctgccaagaaaaacaagtggcttactgcccttctggcaagcctgaaggcctgaactatgcctgtctcactcacagtggatatggagatgggtctgattaatagcgttgtttgggaaatagagagttgagataaacactctcattcagtagttactgaaagaaaactctgctagaatgataaatgtcatggtggtctataactccaaataaacaatgcaacgttcc

SEQ ID NO: 3 - protein, 724 aa, Artificial Sequence
  SLC34A2-ROS(L)
MAPWPELGDAQPNPDKYLEGAAGQQPTAPDKSKETNKTDNTEAPVTKIELLPSYSTATLIDEPTEVDDPWNLPTLQDSGIKWSERDTKGKILCFFQGIGRLILLLGFLYFFVCSLDILSSAFQLVGAGVPNKPGIPKLLEGSKNSIQWEKAEDNGCRITYYILEIRKSTSNNLQNQNLRWKMTFNGSCSSVCTWKSKNLKGIFQFRVVAANNLGFGEYSGISENIILVGDDFWIPETSFILTIIVGIFLVVTIPLTFVWHRRLKNQKSAKEGVTVLINEDKELAELRGLAAGVGLANACYAIHTLPTQEEIENLPAFPREKLTLRLLLGSGAFGEVYEGTAVDILGVGSGEIKVAVKTLKKGSTDQEKIEFLKEAHLMSKFNHPNILKQLGVCLLNEPQYIILELMEGGDLLTYLRKARMATFYGPLLTLVDLVDLCVDISKGCVYLERMHFIHRDLAARNCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQDQLQLFRNFFLNSIYKSRDEANNSGVINESFEGEDGDVICLNSDDIMPVALMETKNREGLNYMVLATECGQGEEKSEGPLGSQESESCGLRKEEKEPHADKDFCQEKQVAYCPSGKPEGLNYACLTHSGYGDGSD

SEQ ID NO: 4 - DNA, 2175 bp, Artificial Sequence
  SLC34A2-ROS(L)
atggctccctggcctgaattgggagatgcccagcccaaccccgataagtacctcgaaggggccgcaggtcagcagcccactgcccctgataaaagcaaagagaccaacaaaacagataacactgaggcacctgtaaccaagattgaacttctgccgtcctactccacggctacactgatagatgagcccactgaggtggatgacccctggaacctacccactcttcaggactcggggatcaagtggtcagagagagacaccaaagggaagattctctgtttcttccaagggattgggagattgattttacttctcggatttctctactttttcgtgtgctccctggatattcttagtagcgccttccagctggttggagctggagtcccaaataaaccaggcattcccaaattactagaagggagtaaaaattcaatacagtgggagaaagctgaagataatggatgtagaattacatactatatccttgagataagaaagagcacttcaaataatttacagaaccagaatttaaggtggaagatgacatttaatggatcctgcagtagtgtttgcacatggaagtccaaaaacctgaaaggaatatttcagttcagagtagtagctgcaaataatctagggtttggtgaatatagtggaatcagtgagaatattatattagttggagatgatttttggataccagaaacaagtttcatacttactattatagttggaatatttctggttgttacaatcccactgacctttgtctggcatagaagattaaagaatcaaaaaagtgccaaggaaggggtgacagtgcttataaacgaagacaaagagttggctgagctgcgaggtctggcagccggagtaggcctggctaatgcctgctatgcaatacatactcttccaacccaagaggagattgaaaatcttcctgccttccctcgggaaaaactgactctgcgtctcttgctgggaagtggagcctttggagaagtgtatgaaggaacagcagtggacatcttaggagttggaagtggagaaatcaaagtagcagtgaagactttgaagaagggttccacagaccaggagaagattgaattcctgaaggaggcacatctgatgagcaaatttaatcatcccaacattctgaagcagcttggagtttgtctgctgaatgaaccccaatacattatcctggaactgatggagggaggagaccttcttacttatttgcgtaaagcccggatggcaacgttttatggtcctttactcaccttggttgaccttgtagacctgtgtgtagatatttcaaaaggctgtgtctacttggaacggatgcatttcattcacagggatctggcagctagaaattgccttgtttccgtgaaagactataccagtccacggatagtgaagattggagactttggactcgccagagacatctataaaaatgattactatagaaagagaggggaaggcctgctcccagttcggtggatggctccagaaagtttgatggatggaatcttcactactcaatctgatgtatggtcttttggaattctgatttgggagattttaactcttggtcatcagccttatccagctcattccaaccttgatgtgttaaactatgtgcaaacaggagggagactggagccaccaagaaattgtcctgatgatctgtggaatttaatgacccagtgctgggctcaagaacccgaccaaagacctacttttcatagaattcaggaccaacttcagttattcagaaattttttcttaaatagcatttataagtccagagatgaagcaaacaacagtggagtcataaatgaaagctttgaaggtgaagatggcgatgtgatttgtttgaattcagatgacattatgccagttgctttaatggaaacgaagaaccgagaagggttaaactatatggtacttgctacagaatgtggccaaggtgaagaaaagtctgagggtcctctaggctcccaggaatctgaatcttgtggtctgaggaaagaagagaaggaaccacatgcagacaaagatttctgccaagaaaaacaagtggcttactgcccttctggcaagcctgaaggcctgaactatgcctgtctcactcacagtggatatggagatgggtctgattaa

SEQ ID NO: 5 - protein, 621 aa, Artificial Sequence
  SLC34A2-ROS(S)
MAPWPELGDAQPNPDKYLEGAAGQQPTAPDKSKETNKTDNTEAPVTKIELLPSYSTATLIDEPTEVDDPWNLPTLQDSGIKWSERDTKGKILCFFQGIGRLILLLGFLYFFVCSLDILSSAFQLVGDDFWIPETSFILTIIVGIFLVVTIPLTFVWHRRLKNQKSAKEGVTVLINEDKELAELRGLAAGVGLANACYAIHTLPTQEEIENLPAFPREKLTLRLLLGSGAFGEVYEGTAVDILGVGSGEIKVAVKTLKKGSTDQEKIEFLKEAHLMSKFNHPNILKQLGVCLLNEPQYIILELMEGGDLLTYLRKARMATFYGPLLTLVDLVDLCVDISKGCVYLERMHFIHRDLAARNCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQDQLQLFRNFFLNSIYKSRDEANNSGVINESFEGEDGDVICLNSDDIMPVALMETKNREGLNYMVLATECGQGEEKSEGPLGSQESESCGLRKEEKEPHADKDFCQEKQVAYCPSGKPEGLNYACLTHSGYGDGSD

SEQ ID NO: 6 - DNA, 1866 bp, Artificial Sequence
  SLC34A2-ROS(S)
atggctccctggcctgaattgggagatgcccagcccaaccccgataagtacctcgaaggggccgcaggtcagcagcccactgcccctgataaaagcaaagagaccaacaaaacagataacactgaggcacctgtaaccaagattgaacttctgccgtcctactccacggctacactgatagatgagcccactgaggtggatgacccctggaacctacccactcttcaggactcggggatcaagtggtcagagagagacaccaaagggaagattctctgtttcttccaagggattgggagattgattttacttctcggatttctctactttttcgtgtgctccctggatattcttagtagcgccttccagctggttggagatgatttttggataccagaaacaagtttcatacttactattatagttggaatatttctggttgttacaatcccactgacctttgtctggcatagaagattaaagaatcaaaaaagtgccaaggaaggggtgacagtgcttataaacgaagacaaagagttggctgagctgcgaggtctggcagccggagtaggcctggctaatgcctgctatgcaatacatactcttccaacccaagaggagattgaaaatcttcctgccttccctcgggaaaaactgactctgcgtctcttgctgggaagtggagcctttggagaagtgtatgaaggaacagcagtggacatcttaggagttggaagtggagaaatcaaagtagcagtgaagactttgaagaagggttccacagaccaggagaagattgaattcctgaaggaggcacatctgatgagcaaatttaatcatcccaacattctgaagcagcttggagtttgtctgctgaatgaaccccaatacattatcctggaactgatggagggaggagaccttcttacttatttgcgtaaagcccggatggcaacgttttatggtcctttactcaccttggttgaccttgtagacctgtgtgtagatatttcaaaaggctgtgtctacttggaacggatgcatttcattcacagggatctggcagctagaaattgccttgtttccgtgaaagactataccagtccacggatagtgaagattggagactttggactcgccagagacatctataaaaatgattactatagaaagagaggggaaggcctgctcccagttcggtggatggctccagaaagtttgatggatggaatcttcactactcaatctgatgtatggtcttttggaattctgatttgggagattttaactcttggtcatcagccttatccagctcattccaaccttgatgtgttaaactatgtgcaaacaggagggagactggagccaccaagaaattgtcctgatgatctgtggaatttaatgacccagtgctgggctcaagaacccgaccaaagacctacttttcatagaattcaggaccaacttcagttattcagaaattttttcttaaatagcatttataagtccagagatgaagcaaacaacagtggagtcataaatgaaagctttgaaggtgaagatggcgatgtgatttgtttgaattcagatgacattatgccagttgctttaatggaaacgaagaaccgagaagggttaaactatatggtacttgctacagaatgtggccaaggtgaagaaaagtctgagggtcctctaggctcccaggaatctgaatcttgtggtctgaggaaagaagagaaggaaccacatgcagacaaagatttctgccaagaaaaacaagtggcttactgcccttctggcaagcctgaaggcctgaactatgcctgtctcactcacagtggatatggagatgggtctgattaa

SEQ ID NO: 7 - protein, 24 aa, Artificial Sequence
  Synthetic Peptide
GCAGCTCAGCCAACTCTTTGTCTT

SEQ ID NO: 8 - protein, 703 aa, Artificial Sequence
  CD74-ROS
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKDDFWIPETSFILTIIVGIFLVVTIPLTFVWHRRLKNQKSAKEGVTVLINEDKELAELRGLAAGVGLANACYAIHTLPTQEEIENLPAFPREKLTLRLLLGSGAFGEVYEGTAVDILGVGSGEIKVAVKTLKKGSTDQEKIEFLKEAHLMSKFNHPNILKQLGVCLLNEPQYIILELMEGGDLLTYLRKARMATFYGPLLTLVDLVDLCVDISKGCVYLERMHFIHRDLAARNCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQDQLQLFRNFFLNSIYKSRDEANNSGVINESFEGEDGDVICLNSDDIMPVALMETKNREGLNYMVLATECGQGEEKSEGPLGSQESESCGLRKEEKEPHADKDFCQEKQVAYCPSGKPEGLNYACLTHSGYGDGSD

SEQ ID NO: 9 - DNA, 2112 bp, Artificial Sequence
  CD74-ROS
atgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagatgatttttggataccagaaacaagtttcatacttactattatagttggaatatttctggttgttacaatcccactgacctttgtctggcatagaagattaaagaatcaaaaaagtgccaaggaaggggtgacagtgcttataaacgaagacaaagagttggctgagctgcgaggtctggcagccggagtaggcctggctaatgcctgctatgcaatacatactcttccaacccaagaggagattgaaaatcttcctgccttccctcgggaaaaactgactctgcgtctcttgctgggaagtggagcctttggagaagtgtatgaaggaacagcagtggacatcttaggagttggaagtggagaaatcaaagtagcagtgaagactttgaagaagggttccacagaccaggagaagattgaattcctgaaggaggcacatctgatgagcaaatttaatcatcccaacattctgaagcagcttggagtttgtctgctgaatgaaccccaatacattatcctggaactgatggagggaggagaccttcttacttatttgcgtaaagcccggatggcaacgttttatggtcctttactcaccttggttgaccttgtagacctgtgtgtagatatttcaaaaggctgtgtctacttggaacggatgcatttcattcacagggatctggcagctagaaattgccttgtttccgtgaaagactataccagtccacggatagtgaagattggagactttggactcgccagagacatctataaaaatgattactatagaaagagaggggaaggcctgctcccagttcggtggatggctccagaaagtttgatggatggaatcttcactactcaatctgatgtatggtcttttggaattctgatttgggagattttaactcttggtcatcagccttatccagctcattccaaccttgatgtgttaaactatgtgcaaacaggagggagactggagccaccaagaaattgtcctgatgatctgtggaatttaatgacccagtgctgggctcaagaacccgaccaaagacctacttttcatagaattcaggaccaacttcagttattcagaaattttttcttaaatagcatttataagtccagagatgaagcaaacaacagtggagtcataaatgaaagctttgaaggtgaagatggcgatgtgatttgtttgaattcagatgacattatgccagttgctttaatggaaacgaagaaccgagaagggttaaactatatggtacttgctacagaatgtggccaaggtgaagaaaagtctgagggtcctctaggctcccaggaatctgaatcttgtggtctgaggaaagaagagaaggaaccacatgcagacaaagatttctgccaagaaaaacaagtggcttactgcccttctggcaagcctgaaggcctgaactatgcctgtctcactcacagtggatatggagatgggtctgattaa

SEQ ID NO: 10 - protein, 592 aa, Artificial Sequence
  SLC34A2-ROS(VS)
MAPWPELGDAQPNPDKYLEGAAGQQPTAPDKSKETNKTDNTEAPVTKIELLPSYSTATLIDEPTEVDDPWNLPTLQDSGIKWSERDTKGKILCFFQGIGRLILLLGFLYFFVCSLDILSSAFQLVGVWHRRLKNQKSAKEGVTVLINEDKELAELRGLAAGVGLANACYAIHTLPTQEEIENLPAFPREKLTLRLLLGSGAFGEVYEGTAVDILGVGSGEIKVAVKTLKKGSTDQEKIEFLKEAHLMSKFNHPNILKQLGVCLLNEPQYIILELMEGGDLLTYLRKARMATFYGPLLTLVDLVDLCVDISKGCVYLERMHFIHRDLAARCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQDQLQLFRNFFLNSIYKSRDEANNSGVINESFEGEDGDVICLNSDDIMPVALMETKNREGLNYMVLATECGQGEEKSEGPLGSQESESCGLRKEEKEPHADKDFCQEKQVAYCPSGKPEGLNYACLTHSGYGDGSD

SEQ ID NO: 11 - DNA, 1782 bp, Artificial Sequence
  SLC34A2-ROS(VS)
atggctccctggcctgaattgggagatgcccagcccaaccccgataagtacctcgaaggggccgcaggtcagcagcccactgcccctgataaaagcaaagagaccaacaaaacagataacactgaggcacctgtaaccaagattgaacttctgccgtcctactccacggctacactgatagatgagcccactgaggtggatgacccctggaacctacccactcttcaggactcggggatcaagtggtcagagagagacaccaaagggaagattctctgtttcttccaagggattgggagattgattttacttctcggatttctctactttttcgtgtgctccctggatattcttagtagcgccttccagctggttggagtctggcatagaagattaaagaatcaaaaaagtgccaaggaaggggtgacagtgcttataaacgaagacaaagagttggctgagctgcgaggtctggcagccggagtaggcctggctaatgcctgctatgcaatacatactcttccaacccaagaggagattgaaaatcttcctgccttccctcgggaaaaactgactctgcgtctcttgctgggaagtggagcctttggagaagtgtatgaaggaacagcagtggacatcttaggagttggaagtggagaaatcaaagtagcagtgaagactttgaagaagggttccacagaccaggagaagattgaattcctgaaggaggcacatctgatgagcaaatttaatcatcccaacattctgaagcagcttggagtttgtctgctgaatgaaccccaatacattatcctggaactgatggagggaggagaccttcttacttatttgcgtaaagcccggatggcaacgttttatggtcctttactcaccttggttgaccttgtagacctgtgtgtagatatttcaaaaggctgtgtctacttggaacggatgcatttcattcacagggatctggcagctagaaattgccttgtttccgtgaaagactataccagtccacggatagtgaagattggagactttggactcgccagagacatctataaaaatgattactatagaaagagaggggaaggcctgctcccagttcggtggatggctccagaaagtttgatggatggaatcttcactactcaatctgatgtatggtcttttggaattctgatttgggagattttaactcttggtcatcagccttatccagctcattccaaccttgatgtgttaaactatgtgcaaacaggagggagactggagccaccaagaaattgtcctgatgatctgtggaatttaatgacccagtgctgggctcaagaacccgaccaaagacctacttttcatagaattcaggaccaacttcagttattcagaaattttttcttaaatagcatttataagtccagagatgaagcaaacaacagtggagtcataaatgaaagctttgaaggtgaagatggcgatgtgatttgtttgaattcagatgacattatgccagttgctttaatggaaacgaagaaccgagaagggttaaactatatggtacttgctacagaatgtggccaaggtgaagaaaagtctgagggtcctctaggctcccaggaatctgaatcttgtggtctgaggaaagaagagaaggaaccacatgcagacaaagatttctgccaagaaaaacaagtggcttactgcccttctggcaagcctgaaggcctgaactatgcctgtctcactcacagtggatatggagatgggtctgattaa

SEQ ID NO: 12 - protein, 34 aa, Artificial Sequence
  Synthetic Peptide
FEMSRHSLEQKPTDAPPKDDFWIPETSFILTIIV

SEQ ID NO: 13 - DNA, 21 bp, Artificial Sequence
  Synthetic Polynucleotide
aagcccggauggcaacguutt

SEQ ID NO: 14 - DNA, 21 bp, Artificial Sequence
  Synthetic Polynucleotide
aagccugaaggccugaacutt

SEQ ID NO: 15 - DNA, 24 bp, Artificial Sequence
  Synthetic Polynucleotide
cagcaagagacgcagagtcagttt

SEQ ID NO: 16 - DNA, 26 bp, Artificial Sequence
  Synthetic Polynucleotide
gctgttctccaggctgaagtatatgg

SEQ ID NO: 17 - DNA, 24 bp, Artificial Sequence
  Synthetic Polynucleotide
gtaaccctggtgctagttgcaaag

SEQ ID NO: 18 - DNA, 2637 bp, Artificial Sequence
  FIG-ROS(L)
atgtcggcgggcggtccatgcccagcagcagccggagggggcccagggggcgcctcctgctccgtgggggcccctggcggggtatccatgttccggtggctggaggtgctggagaaggagttcgacaaagcttttgtggatgtggatctgctcctgggagagatcgatccagaccaagcggacatcacttatgaggggcgacagaagatgaccagcctgagctcctgctttgcacagctttgccacaaagcccagtctgtgtctcaaatcaaccacaagctggaggcacagttggtggatctgaaatctgaactgacagaaacccaagcagagaaagttgttttggagaaagaagtacatgatcagcttttacagctgcactctattcagctgcagcttcatgctaaaactggtcaaagtgctgactctggtaccattaaggcaaaattggaaagagagcttgaggcaaacaaaaaagaaaaaatgaaagaagcacaacttgaagctgaagtgaaattgttgagaaaagagaatgaagcccttcgtagacatatagctgttctccaggctgaagtatatggggcgagactagctgccaagtacttggataaggaactggcaggaagggtccaacagatacaattgctaggacgagatatgaagggacctgctcatgataagctttggaaccaattagaagctgaaatacatttgcatcgtcacaaaactgtgatccgagcctgcagaggacgtaatgacttgaaacgaccaatgcaagcaccaccaggccatgatcaagattccctaaagaaaagccaaggtgttggtccaattagaaaagttctcctccttaaggaagatcatgaaggccttggcatttcaattacaggtgggaaagaacatggtgttccaatcctcatctctgagatccatccggggcaacctgctgatagatgcggagggctgcacgttggggatgctattttggcagtcaacggagttaacctaagggacacaaagcataaagaagctgtaactattctttctcagcagagaggagagattgaatttgaagtagtttatgtggctcctgaagtggattctgatgatgaaaacgtagagtatgaagatgagagtggacatcgttaccgtttgtaccttgatgagttagaaggaggtggtaaccctggtgctagttgcaaagacacaagtggggaaatcaaagtattacaagtctggcatagaagattaaagaatcaaaaaagtgccaaggaaggggtgacagtgcttataaacgaagacaaagagttggctgagctgcgaggtctggcagccggagtaggcctggctaatgcctgctatgcaatacatactcttccaacccaagaggagattgaaaatcttcctgccttccctcgggaaaaactgactctgcgtctcttgctgggaagtggagcctttggagaagtgtatgaaggaacagcagtggacatcttaggagttggaagtggagaaatcaaagtagcagtgaagactttgaagaagggttccacagaccaggagaagattgaattcctgaaggaggcacatctgatgagcaaatttaatcatcccaacattctgaagcagcttggagtttgtctgctgaatgaaccccaatacattatcctggaactgatggagggaggagaccttcttacttatttgcgtaaagcccggatggcaacgttttatggtcctttactcaccttggttgaccttgtagacctgtgtgtagatatttcaaaaggctgtgtctacttggaacggatgcatttcattcacagggatctggcagctagaaattgccttgtttccgtgaaagactataccagtccacggatagtgaagattggagactttggactcgccagagacatctataaaaatgattactatagaaagagaggggaaggcctgctcccagttcggtggatggctccagaaagtttgatggatggaatcttcactactcaatctgatgtatggtcttttggaattctgatttgggagattttaactcttggtcatcagccttatccagctcattccaaccttgatgtgttaaactatgtgcaaacaggagggagactggagccaccaagaaattgtcctgatgatctgtggaatttaatgacccagtgctgggctcaagaacccgaccaaagacctacttttcatagaattcaggaccaacttcagttattcagaaattttttcttaaatagcatttataagtccagagatgaagcaaacaacagtggagtcataaatgaaagctttgaaggtgaagatggcgatgtgatttgtttgaattcagatgacattatgccagttgctttaatggaaacgaagaaccgagaagggttaaactatatggtacttgctacagaatgtggccaaggtgaagaaaagtctgagggtcctctaggctcccaggaatctgaatcttgtggtctgaggaaagaagagaaggaaccacatgcagacaaagatttctgccaagaaaaacaagtggcttactgcccttctggcaagcctgaaggcctgaactatgcctgtctcactcacagtggatatggagatgggtctgattaa

SEQ ID NO: 19 - protein, 878 aa, Artificial Sequence
  FIG-ROS(L)
MSAGGPCPAAAGGGPGGASCSVGAPGGVSMFRWLEVLEKEFDKAFVDVDLLLGEIDPDQADITYEGRQKMTSLSSCFAQLCHKAQSVSQINHKLEAQLVDLKSELTETQAEKVVLEKEVHDQLLQLHSIQLQLHAKTGQSADSGTIKAKLERELEANKKEKMKEAQLEAEVKLLRKENEALRRHIAVLQAEVYGARLAAKYLDKELAGRVQQIQLLGRDMKGPAHDKLWNQLEAEIHLHRHKTVIRACRGRNDLKRPMQAPPGHDQDSLKKSQGVGPIRKVLLLKEDHEGLGISITGGKEHGVPILISEIHPGQPADRCGGLHVGDAILAVNGVNLRDTKHKEAVTILSQQRGEIEFEVVYVAPEVDSDDENVEYEDESGHRYRLYLDELEGGGNPGASCKDTSGEIKVLQVWHRRLKNQKSAKEGVTVLINEDKELAELRGLAAGVGLANACYAIHTLPTQEEIENLPAFPREKLTLRLLLGSGAFGEVYEGTAVDILGVGSGEIKVAVKTLKKGSTDQEKIEFLKEAHLMSKFNHPNILKQLGVCLLNEPQYIILELMEGGDLLTYLRKARMATFYGPLLTLVDLVDLCVDISKGCVYLERMHFIHRDLAARNCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQDQLQLFRNFFLNSIYKSRDEANNSGVINESFEGEDGDVICLNSDDIMPVALMETKNREGLNYMVLATECGQGEEKSEGPLGSQESESCGLRKEEKEPHADKDFCQEKQVAYCPSGKPEGLNYACLTHSGYGDGSD

SEQ ID NO: 20 - DNA, 1893 bp, Artificial Sequence
  FIG-ROS(S)
atgtcggcgggcggtccatgcccagcagcagccggagggggcccagggggcgcctcctgctccgtgggggcccctggcggggtatccatgttccggtggctggaggtgctggagaaggagttcgacaaagcttttgtggatgtggatctgctcctgggagagatcgatccagaccaagcggacatcacttatgaggggcgacagaagatgaccagcctgagctcctgctttgcacagctttgccacaaagcccagtctgtgtctcaaatcaaccacaagctggaggcacagttggtggatctgaaatctgaactgacagaaacccaagcagagaaagttgttttggagaaagaagtacatgatcagcttttacagctgcactctattcagctgcagcttcatgctaaaactggtcaaagtgctgactctggtaccattaaggcaaaattggaaagagagcttgaggcaaacaaaaaagaaaaaatgaaagaagcacaacttgaagctgaagtgaaattgttgagaaaagagaatgaagcccttcgtagacatatagctgttctccaggctgaagtatatggggcgagactagctgccaagtacttggataaggaactggcaggaagtactcttccaacccaagaggagattgaaaatcttcctgccttccctcgggaaaaactgactctgcgtctcttgctgggaagtggagcctttggagaagtgtatgaaggaacagcagtggacatcttaggagttggaagtggagaaatcaaagtagcagtgaagactttgaagaagggttccacagaccaggagaagattgaattcctgaaggaggcacatctgatgagcaaatttaatcatcccaacattctgaagcagcttggagtttgtctgctgaatgaaccccaatacattatcctggaactgatggagggaggagaccttcttacttatttgcgtaaagcccggatggcaacgttttatggtcctttactcaccttggttgaccttgtagacctgtgtgtagatatttcaaaaggctgtgtctacttggaacggatgcatttcattcacagggatctggcagctagaaattgccttgtttccgtgaaagactataccagtccacggatagtgaagattggagactttggactcgccagagacatctataaaaatgattactatagaaagagaggggaaggcctgctcccagttcggtggatggctccagaaagtttgatggatggaatcttcactactcaatctgatgtatggtcttttggaattctgatttgggagattttaactcttggtcatcagccttatccagctcattccaaccttgatgtgttaaactatgtgcaaacaggagggagactggagccaccaagaaattgtcctgatgatctgtggaatttaatgacccagtgctgggctcaagaacccgaccaaagacctacttttcatagaattcaggaccaacttcagttattcagaaattttttcttaaatagcatttataagtccagagatgaagcaaacaacagtggagtcataaatgaaagctttgaaggtgaagatggcgatgtgatttgtttgaattcagatgacattatgccagttgctttaatggaaacgaagaaccgagaagggttaaactatatggtacttgctacagaatgtggccaaggtgaagaaaagtctgagggtcctctaggctcccaggaatctgaatcttgtggtctgaggaaagaagagaaggaaccacatgcagacaaagatttctgccaagaaaaacaagtggcttactgcccttctggcaagcctgaaggcctgaactatgcctgtctcactcacagtggatatggagatgggtctgattaa

SEQ ID NO: 21 - protein, 630 aa, Artificial Sequence
  FIG-ROS(S)
MSAGGPCPAAAGGGPGGASCSVGAPGGVSMFRWLEVLEKEFDKAFVDVDLLLGEIDPDQADITYEGRQKMTSLSSCFAQLCHKAQSVSQINHKLEAQLVDLKSELTETQAEKVVLEKEVHDQLLQLHSIQLQLHAKTGQSADSGTIKAKLERELEANKKEKMKEAQLEAEVKLLRKENEALRRHIAVLQAEVYGARLAAKYLDKELAGSTLPTQEEIENLPAFPREKLTLRLLLGSGAFGEVYEGTAVDILGVGSGEIKVAVKTLKKGSTDQEKIEFLKEAHLMSKFNHPNILKQLGVCLLNEPQYIILELMEGGDLLTYLRKARMATFYGPLLTLVDLVDLCVDISKGCVYLERMHFIHRDLAARNCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQDQLQLFRNFFLNSIYKSRDEANNSGVINESFEGEDGDVICLNSDDIMPVALMETKNREGLNYMVLATECGQGEEKSEGPLGSQESESCGLRKEEKEPHADKDFCQEKQVAYCPSGKPEGLNYACLTHSGYGDGSD

SEQ ID NO: 22 - DNA, 3030 bp, Artificial Sequence
  FIG-ROS(XL)
atgtcggcgggcggtccatgcccagcagcagccggagggggcccagggggcgcctcctgctccgtgggggcccctggcggggtatccatgttccggtggctggaggtgctggagaaggagttcgacaaagcttttgtggatgtggatctgctcctgggagagatcgatccagaccaagcggacatcacttatgaggggcgacagaagatgaccagcctgagctcctgctttgcacagctttgccacaaagcccagtctgtgtctcaaatcaaccacaagctggaggcacagttggtggatctgaaatctgaactgacagaaacccaagcagagaaagttgttttggagaaagaagtacatgatcagcttttacagctgcactctattcagctgcagcttcatgctaaaactggtcaaagtgctgactctggtaccattaaggcaaaattggaaagagagcttgaggcaaacaaaaaagaaaaaatgaaagaagcacaacttgaagctgaagtgaaattgttgagaaaagagaatgaagcccttcgtagacatatagctgttctccaggctgaagtatatggggcgagactagctgccaagtacttggataaggaactggcaggaagggtccaacagatacaattgctaggacgagatatgaagggacctgctcatgataagctttggaaccaattagaagctgaaatacatttgcatcgtcacaaaactgtgatccgagcctgcagaggacgtaatgacttgaaacgaccaatgcaagcaccaccaggccatgatcaagattccctaaagaaaagccaaggtgttggtccaattagaaaagttctcctccttaaggaagatcatgaaggccttggcatttcaattacaggtgggaaagaacatggtgttccaatcctcatctctgagatccatccggggcaacctgctgatagatgcggagggctgcacgttggggatgctattttggcagtcaacggagttaacctaagggacacaaagcataaagaagctgtaactattctttctcagcagagaggagagattgaatttgaagtagtttatgtggctcctgaagtggattctgatgatgaaaacgtagagtatgaagatgagagtggacatcgttaccgtttgtaccttgatgagttagaaggaggtggtaaccctggtgctagttgcaaagacacaagtggggaaatcaaagtattacaagctggagtcccaaataaaccaggcattcccaaattactagaagggagtaaaaattcaatacagtgggagaaagctgaagataatggatgtagaattacatactatatccttgagataagaaagagcacttcaaataatttacagaaccagaatttaaggtggaagatgacatttaatggatcctgcagtagtgtttgcacatggaagtccaaaaacctgaaaggaatatttcagttcagagtagtagctgcaaataatctagggtttggtgaatatagtggaatcagtgagaatattatattagttggagatgatttttggataccagaaacaagtttcatacttactattatagttggaatatttctggttgttacaatcccactgacctttgtctggcatagaagattaaagaatcaaaaaagtgccaaggaaggggtgacagtgcttataaacgaagacaaagagttggctgagctgcgaggtctggcagccggagtaggcctggctaatgcctgctatgcaatacatactcttccaacccaagaggagattgaaaatcttcctgccttccctcgggaaaaactgactctgcgtctcttgctgggaagtggagcctttggagaagtgtatgaaggaacagcagtggacatcttaggagttggaagtggagaaatcaaagtagcagtgaagactttgaagaagggttccacagaccaggagaagattgaattcctgaaggaggcacatctgatgagcaaatttaatcatcccaacattctgaagcagcttggagtttgtctgctgaatgaaccccaatacattatcctggaactgatggagggaggagaccttcttacttatttgcgtaaagcccggatggcaacgttttatggtcctttactcaccttggttgaccttgtagacctgtgtgtagatatttcaaaaggctgtgtctacttggaacggatgcatttcattcacagggatctggcagctagaaattgccttgtttccgtgaaagactataccagtccacggatagtgaagattggagactttggactcgccagagacatctataaaaatgattactatagaaagagaggggaaggcctgctcccagttcggtggatggctccagaaagtttgatggatggaatcttcactactcaatctgatgtatggtcttttggaattctgatttgggagattttaactcttggtcatcagccttatccagctcattccaaccttgatgtgttaaactatgtgcaaacaggagggagactggagccaccaagaaattgtcctgatgatctgtggaatttaatgacccagtgctgggctcaagaacccgaccaaagacctacttttcatagaattcaggaccaacttcagttattcagaaattttttcttaaatagcatttataagtccagagatgaagcaaacaacagtggagtcataaatgaaagctttgaaggtgaagatggcgatgtgatttgtttgaattcagatgacattatgccagttgctttaatggaaacgaagaaccgagaagggttaaactatatggtacttgctacagaatgtggccaaggtgaagaaaagtctgagggtcctctaggctcccaggaatctgaatcttgtggtctgaggaaagaagagaaggaaccacatgcagacaaagatttctgccaagaaaaacaagtggcttactgcccttctggcaagcctgaaggcctgaactatgcctgtctcactcacagtggatatggagatgggtctgattaa

SEQ ID NO: 23 - protein, 1009 aa, Artificial Sequence
  FIG-ROS(XL)
MSAGGPCPAAAGGGPGGASCSVGAPGGVSMFRWLEVLEKEFDKAFVDVDLLLGEIDPDQADITYEGRQKMTSLSSCFAQLCHKAQSVSQINHKLEAQLVDLKSELTETQAEKVVLEKEVHDQLLQLHSIQLQLHAKTGQSADSGTIKAKLERELEANKKEKMKEAQLEAEVKLLRKENEALRRHIAVLQAEVYGARLAAKYLDKELAGRVQQIQLLGRDMKGPAHDKLWNQLEAEIHLHRHKTVIRACRGRNDLKRPMQAPPGHDQDSLKKSQGVGPIRKVLLLKEDHEGLGISITGGKEHGVPILISEIHPGQPADRCGGLHVGDAILAVNGVNLRDTKHKEAVTILSQQRGEIEFEVVYVAPEVDSDDENVEYEDESGHRYRLYLDELEGGGNPGASCKDTSGEIKVLQAGVPNKPGIPKLLEGSKNSIQWEKAEDNGCRITYYILEIRKSTSNNLQNQNLRWKMTFNGSCSSVCTWKSKNLKGIFQFRVVAANNLGFGEYSGISENIILVGDDFWIPETSFILTIIVGIFLVVTIPLTFVWHRRLKNQKSAKEGVTVLINEDKELAELRGLAAGVGLANACYAIHTLPTQEEIENLPAFPREKLTLRLLLGSGAFGEVYEGTAVDILGVGSGEIKVAVKTLKKGSTDQEKIEFLKEAHLMSKFNHPNILKQLGVCLLNEPQYIILELMEGGDLLTYLRKARMATFYGPLLTLVDLVDLCVDISKGCVYLERMHFIHRDLAARNCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQDQLQLFRNFFLNSIYKSRDEANNSGVINESFEGEDGDVICLNSDDIMPVALMETKNREGLNYMVLATECGQGEEKSEGPLGSQESESCGLRKEEKEPHADKDFCQEKQVAYCPSGKPEGLNYACLTHSGYGDGSD

SEQ ID NO: 24 - protein, 278 aa, Artificial Sequence
  Synthetic Peptide
LTLRLLLGSGAFGEVYEGTAVDILGVGSGEIKVAVKTLKKGSTDQEKIEFLKEAHLMSKFNHPNILKQLGVCLLNEPQYIILELMEGGDLLTYLRKARMATFYGPLLTLVDLVDLCVDISKGCVYLERMHFIHRDLAARNCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQDQLQLFRNFF

SEQ ID NO: 25 - protein, 6 aa, Artificial Sequence
  Synthetic Peptide
VGVWHR

SEQ ID NO: 26 - protein, 6 aa, Artificial Sequence
  Synthetic Peptide
LVGDDF

SEQ ID NO: 27 - protein, 6 aa, Artificial Sequence
  Synthetic Peptide
LVGAGV

SEQ ID NO: 28 - protein, 6 aa, Artificial Sequence
  Synthetic Peptide
PPKDDF

SEQ ID NO: 29 - protein, 6 aa, Artificial Sequence
  Synthetic Peptide
AGSTLP

SEQ ID NO: 30 - protein, 6 aa, Artificial Sequence
  Synthetic Peptide
LQVWHR

SEQ ID NO: 31 - protein, 6 aa, Artificial Sequence
  Synthetic Peptide
VLQAGV

SEQ ID NO: 32 - protein, 1620 aa, Artificial Sequence
  Human ALK
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 33 - DNA, 6222 bp, Artificial Sequence
  Human ALK
gggggcggcagcggtggtagcagctggtacctcccgccgcctctgttcggagggtcgcggggcaccgaggtgctttccggccgccctctggtcggccacccaaagccgcgggcgctgatgatgggtgaggagggggcggcaagatttcgggcgcccctgccctgaacgccctcagctgctgccgccggggccgctccagtgcctgcgaactctgaggagccgaggcgccggtgagagcaaggacgctgcaaacttgcgcagcgcgggggctgggattcacgcccagaagttcagcaggcagacagtccgaagccttcccgcagcggagagatagcttgagggtgcgcaagacggcagcctccgccctcggttcccgcccagaccgggcagaagagcttggaggagccaaaaggaacgcaaaaggcggccaggacagcgtgcagcagctgggagccgccgttctcagccttaaaagttgcagagattggaggctgccccgagaggggacagaccccagctccgactgcggggggcaggagaggacggtacccaactgccacctcccttcaaccatagtagttcctctgtaccgagcgcagcgagctacagacgggggcgcggcactcggcgcggagagcgggaggctcaaggtcccagccagtgagcccagtgtgcttgagtgtctctggactcgcccctgagcttccaggtctgtttcatttagactcctgctcgcctccgtgcagttgggggaaagcaagagacttgcgcgcacgcacagtcctctggagatcaggtggaaggagccgctgggtaccaaggactgttcagagcctcttcccatctcggggagagcgaagggtgaggctgggcccggagagcagtgtaaacggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaaaa

(19) United States
(12) Patent Application Publication
(10) Pub. No.: US 2017/0275706 A1
(43) Pub. Date: Sep. 28, 2017

(54) ALK AND ROS KINASE IN CANCER

(71) Applicant: CELL SIGNALING TECHNOLOGY, INC., Danvers, MA (US)

(72) Inventors: Victoria McGuinness Rimkunas, Reading, MA (US); Herbert Haack, South Hamilton, MA (US); Katherine Eleanor Crosby, Middleton, MA (US)

(73) Assignee: CELL SIGNALING TECHNOLOGY, INC., Danvers, MA (US)

(21) Appl. No.: 15/616,111

(22) Filed: Jun. 7, 2017

Related U.S. Application Data
(63) Continuation of application No. 13/699,309, filed on May 13, 2013, now abandoned.
(60) Provisional application No. 61/347,251, filed on May 21, 2010.

(51) Int. Cl. C12Q 1/68; G01N 33/574; C07K 16/40; C12Q 1/48

(52) U.S. Cl. CPC C12Q 1/6886; C12Q 1/485; C12Q 2600/136; C07K 16/40; G01N 2333/71; G01N 33/57484; C12Q 2600/106

(57) ABSTRACT

The invention provides a method for identifying a patient with cancer or suspected of having cancer as a patient likely to respond to an ALK- and/or ROS-inhibiting therapeutic, comprising: contacting a biological sample from a patient with a first reagent that specifically binds a polypeptide having ROS kinase activity and a second reagent that specifically binds to a polypeptide having ALK knase activity, and detecting whether the first reagent or the second reagent specifically binds to the biological sample, wherein detection of binding of either the first reagent or the second reagent to the biological sample identifies the patient as a patient likely to respond to an ALK-inhibiting and/or ROS-inhibiting therapeutic.

### CROSS REFERENCE TO RELATED APPLICATIONS

This application is a continuation of U.S. patent application Ser. No. 13/699,309, filed May 13, 2013, which is a National State Entry of International Application No. PCT/US2011/037622, filed May 23, 2011, which claims priority to and benefit of U.S. Provisional Patent Application Ser. No. 61/347,251 filed May 21, 2010, the entire disclosure of which is hereby incorporated by reference.

### INCORPORATION BY REFERENCE OF SEQUENCE LISTING

The Sequence Listing in an ASCII text file, named as 28127Z_CST_308CON_SequenceListing.txt of 122 KB, created on Jun. 7, 2017, and submitted to the United States Patent and Trademark Office via EFS-Web, is incorporated herein by reference.

### BACKGROUND OF THE INVENTION

The invention relates generally to proteins and genes involved in cancer (e.g., human cancer), and to the detection, diagnosis and treatment of cancer.

Many cancers are characterized by disruptions in cellular signaling pathways that lead to aberrant control of cellular processes including growth and proliferation. These disruptions are often caused by changes in the activity of particular signaling proteins, such as kinases.

Aberrant expression of protein kinase proteins can be the causative agent of (and the driver of) cancer. Aberrant expression can be caused by the fusion of the protein (or kinase portion thereof) with a secondary protein (or portion there), expression of a truncated portion of the protein, or by abnormal regulation of expression of the full-length protein.

It is known that gene translocations resulting in kinase fusion proteins with aberrant signaling activity can directly lead to certain cancers (see, e.g., Mitelman et al., Nature Reviews Cancer 7: 233-245, 2007, Futreal et al., Nat Rev Cancer 4(3): 177-183 (2004), and Falini et al., Blood 99(2): 409-426 (2002). For example, the BCR-ABL oncoprotein, a tyrosine kinase fusion protein, is the causative agent and drives human chronic myeloid leukemia (CML). The BCR-ABL oncoprotein, which is found in at least 90-95% of CML cases, is generated by the translocation of gene sequences from the c-ABL protein tyrosine kinase on chromosome 9 into BCR sequences on chromosome 22, producing the so-called Philadelphia chromosome. See, e.g. Kurzock et al., N. Engl. J. Med. 319: 990-998 (1988). The translocation is also observed in acute lymphocytic leukemia (ALL) and acute myeloid leukemia (AML) cases. These discoveries spurred FDA approval of imatinib mesylate (sold under the trademark Gleevec® by Novartis) and dasatinig (sold by Bristol-Mysers Squibb under the trademark Sprycel®), small molecule inhibitors of the ABL kinase, for the treatment of CML and ALL. These drugs are examples of drugs designed to interfere with the signaling pathways that drive the growth of tumor cells. The development of such drugs represents a significant advance over the conventional therapies for CML and ALL, chemotherapy and radiation, which are plagued by well known side-effects and are often of limited effect since they fail to specifically target the underlying causes of the cancer.

Thus, it would be useful to identify proteins that drive cancers in order to detect cancers at an early stage, when they are more likely to respond to therapy. Additionally, identification of such proteins will, among other things, desirably enable new methods for selecting patients for targeted therapies, as well as for the screening and development of new drugs that inhibit such proteins and, thus, treat cancer.

The oncogenic role of receptor tyrosine kinases (RTKs) have been implicated in blood cancers such as lymphoma and leukemia, as well as many types of solid tumor cancers including, lung cancer, colon cancer, liver cancer, brain cancer, and breast cancer. Unfortunately solid tumors cancer is often not diagnosed at an early stage, and it often does not respond completely to surgery even when combined with chemotherapy or radiotherapy. For example, non-small cell lung carcinoma NSCLC is the leading cause of cancer death in the United States, and accounts for about 87% of all lung cancers. See “Cancer Facts and Figures 2005,” American Cancer Society.

Thus, it would be useful to discover new ways to identify cancer at an early stage, and new ways (and new reagents) to treat cancer.

### SUMMARY OF THE INVENTION

The invention is based upon the discovery that ROS and ALK are independent drivers of cancer. In other words, if ROS kinase is the driver of a cancer, ALK will not drive the cancer. Similarly, if ALK kinase is the driver of a cancer, ROS will not drive that cancer. This discovery will allow for identification of patients whose tumors will benefit from therapy with an ALK-inhibiting therapeutic or a ROS-inhibiting therapeutic, or both.

Accordingly, in a first aspect, the invention provides a method of treating a patient for cancer, comprising: detecting the presence in a biological sample from a patient having or suspected of having cancer of a polypeptide selected from the group consisting of a polypeptide having ROS kinase activity and a polypeptide having ALK kinase activity; and administering a therapeutically effective amount of an ALK/ROS-inhibiting therapeutic to the patient, thereby treating the patient for cancer. In some embodiments, the detecting step is performed by using a reagent that specifically binds to either a polypeptide having ROS kinase activity or a polypeptide having ALK kinase activity.

In some embodiments, the detecting step is performed by using a reagent that specifically binds to a polynucleotide encoding either a polypeptide having ROS kinase activity or a polypeptide having ALK kinase activity.

In a further aspect, the invention provides a method for identifying a patient with cancer or suspected of having cancer as a patient likely to respond to an ALK-inhibiting therapeutic, comprising: contacting a biological sample from a patient with a first reagent that specifically binds a polypeptide having ROS kinase activity and a second reagent that specifically binds to a polypeptide having ALK knase activity and detecting whether the first reagent or the second reagent specifically binds to the biological sample, wherein detection of binding of either the first reagent or the second reagent to the biological sample identifies the patient as a patient likely to respond to an ALK-inhibiting therapeutic.

In a further aspect, the invention provides a method for identifying a patient with cancer or suspected of having cancer as a patient likely to respond to an ALK-inhibiting therapeutic, comprising: contacting a biological sample from a patient with a first reagent that specifically binds a polypeptide having ROS kinase activity or specifically binds to a polynucleotide encoding a polypeptide having ROS kinase activity and a second reagent that specifically binds to a polypeptide having ALK knase activity or specifically binds to a polynucleotide encoding a polypeptide having ALK kinase activity and detecting whether the first reagent or the second reagent specifically binds to the biological sample, wherein detection of binding of either the first reagent or the second reagent to the biological sample identifies the patient as a patient likely to respond to an ALK-inhibiting therapeutic.

In various embodiments, the first reagent specifically binds to full length ROS kinase protein. In various embodiments, the second reagent specifically binds to full length ALK kinase protein. In various embodiments, the first reagent specifically binds to the kinase domain of ROS kinase protein. In various embodiments, the second reagent specifically binds to the kinase domain of ALK kinase protein. In some embodiments, the first reagent is an antibody. In some embodiments, the second reagent is an antibody.

In various embodiments of all of the aspect of the invention, the patient is a human patient and the cancer (or suspected cancer) is from a human. In some embodiments, the ROS-inhibiting therapeutic or the ALK-inhibiting therpauetic is PF-02341066, NVT TAE-684, or AP26113. In some embodiments, the ROS-inhibiting therapeutic or ALK-inhibiting therapeutic is AP26113, CEP-14083, CEP-14513, CEP11988, CH5424802, WHI-P131 and WHI-P154.

In various embodiments, the biological sample is from the cancer or suspected cancer of the patient. In some embodiments, the cancer is a solid tumor cancer. In some embodiments, the cancer is leukemia. In some embodiments, the cancer is lymphoma. In some embodiments, the cancer is a lung cancer (e.g., a non-small cell lung carcinoma or a small cell lung carcinoma). In some embodiments, the cancer is a brain cancer (e.g., glioblastoma). In some embodiments, the cancer is a liver cancer (e.g., cholangiocarcinoma). In some embodiments, the cancer is colon cancer. In some embodiments, the cancer is breast cancer. In some embodiments, the cancer is ovarian cancer.

In further embodiments, the biological sample is selected from the group consisting of a tumor biopsy, a bronchoalveolar lavage, a circulating tumor cell, a tumor resection, a fine needle aspirate, a lymph node, a bone marrow sample, and an effusion (e.g., pleural effusion).

In some embodiments, the reagents (e.g., the antibodies) are detectably labeled. In another embodiment, the reagent (or first reagent) specifically binds to a full length ROS polypeptide. In another embodiment, the reagent (or first reagent) specifically binds to a ROS kinase domain. In another embodiment, the reagent (or first reagent) specifically binds to a ROS fusion polypeptide (e.g., specifically binds to a CD74-ROS fusion polypeptide, an SLC34A2-ROS(S) polypeptide, an SLC34A2-ROS(L) polypeptide, an SLC34A2-ROS(VS) polypeptide, a FIG-ROS (L) polypeptide, a FIG-ROS(S) polypeptide, or a FIG-ROS(VL) polypeptide. In various embodiments, the polypeptide having ROS kinase activity (to which the reagent of the invention specifically binds) comprises the amino acid sequence of SEQ ID NO: 1, SEQ ID NO: 24, SEQ ID NO: 21, SEQ ID NO: 19, SEQ ID NO: 23, SEQ ID NO: 10, SEQ ID NO: 5, SEQ ID NO: 3, or SEQ ID NO: 8. These ROS fusions have been previously described (see U.S. Patent Publication Nos. 20100221737 and 20100143918, and PCT Publication No, WO2010.093928, all of which are hereby incorporated by reference in their entirety).

In some embodiments, the reagent (e.g., or first reagent) specifically binds to full length ALK polypeptide. In some embodiments, the reagent specifically binds to an ALK kinase domain. In some embodiments, the reagent (or second reagent) specifically binds to an ALK fusion polypeptide selected from the group consisting of NPM-ALK, ALO17-ALK, TFG-ALK, MSN-ALK, TPM3-ALK, TPM4-ALK, ATIC-ALK, MYH9-ALK, CLTC-ALK, SEC31L1-ALK, RANBP2-ALK, CARS-ALK, EML4-ALK, KIF5B-ALK, and TFG-ALK.

In various embodiments of the methods of the invention, the polypeptide having ROS kinase activity is a full-length ROS polypeptide. In another embodiment, the polypeptide is a ROS fusion polypeptide. In another embodiment, the ROS fusion polypeptide is selected from the group consisting of a CD74-ROS fusion polypeptide, an SLC34A2-ROS(S) polypeptide, an SLC34A2-ROS(L) polypeptide, an SLC34A2-ROS(VS) polypeptide, a FIG-ROS (L) polypeptide, a FIG-ROS(S) polypeptide, and a FIG-ROS(VL) polypeptide. In various embodiments, the polypeptide having ROS kinase activity comprises the amino acid sequence of SEQ ID NO: 1, SEQ ID NO: 24, SEQ ID NO: 21, SEQ ID NO: 19, SEQ ID NO: 23, SEQ ID NO: 10, SEQ ID NO: 5, SEQ ID NO: 3, or SEQ ID NO: 8.

In various embodiments, the polypeptide having ALK kinase activity is full length ALK polypeptide. In another embodiment, the polypeptide is an ALK fusion polypeptide. In another embodiment, the ALK fusion polypeptide is selected from the group consisting of NPM-ALK, ALO17-ALK, TFG-ALK, MSN-ALK, TPM3-ALK, TPM4-ALK, ATIC-ALK, MYH9-ALK, CLTC-ALK, SEC31L1-ALK, RANBP2-ALK, CARS-ALK, EML4-ALK, KIF5B-ALK, and TFG-ALK.

In some embodiments, the method is implemented in a format selected from the group consisting of a flow cytometry assay, an in vitro kinase assay, an immunohistochemistry (IHC) assay, an immunofluorescence (IF) assay, an Enzyme-linked immunosorbent assay (ELISA) assay, and a Western blotting analysis assay.

In one embodiment, the kinase activity of said polypeptide is detected. In another embodiment, the reagent is a heavy-isotope labeled (AQUA) peptide. In another embodiment, the heavy-isotope labeled (AQUA) peptide comprises an amino acid sequence comprising a fusion junction of an ROS fusion polypeptide. In another embodiment, the method is implemented using mass spectrometry analysis.

In some embodiments, where the poynucleotide is detected, the reagent (e.g., or the first reagent and second reagent) is a nucleic acid probe. In some embodiments, the reagent is detectably labeled. In another embodiment, the nucleic acid probe is a fluorescence in-situ hybridization (FISH) probe and said method is implemented in a FISH assay. In another embodiment, the nucleic acid probe is a polymerase chain reaction (PCR) probe and said method is implemented in a PCR assay.

In another aspect, the invention provides a method for inhibiting the progression of a mammalian cancer or suspected mammalian cancer that expresses either a first polypeptide having ROS kinase activity or a second polypeptide having ALK kinase activity, said method comprising the step of inhibiting the expression and/or activity of said first or said second polypeptide in said mammalian cancer or suspected mammalian cancer.

In another aspect, the invention provides a method for inhibiting the progression of a mammalian cancer or suspected mammalian cancer comprising a first polynucleotide encoding a polypeptide having ROS kinase activity or a second polynucleotide encoding a polypeptide having ALK kinase activity, said method comprising the step of inhibiting the expression of said first or said second polynucleotide in said mammalian cancer or suspected mammalian cancer.

In further aspects, the invention provides a method for determining whether a compound inhibits the progression of a mammalian lung cancer or suspected mammalian lung cancer characterized by the expression of a first polypeptide with ROS activity or a second polypeptide with ALK activity, said method comprising the step of determining whether said compound inhibits the expression of said first or said second polypeptide in said cancer. In some embodiments, the cancer is from a human.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-1F—are photographs showing immunohistochemistry and FISH of ROS protein and ROS nucleic acid in non-small cell lung cancer (NSCLC) FFPE tumor tissues. The variation in ROS protein localization are shown as follows: (A) diffuse cytoplasmic with yellow arrows in inset (A) illustrating balanced translocation of the c-ros locus by FISH. (B) Strong punctate localization of ROS in adenocarcinoma with zoom (i.e., enlargened image) in inset. (C) Cytoplasmic localization of ROS staining in large cell carcinoma and corresponding hematoxylin and eosin stain in panel E. (D) Adenocarcinoma with unique cytoplasm staining and membrane localization with zoom in inset showing membrane staining. (E) Hematoxylin and eosin stain corresponding to ROS staining in panel C. (F) Punctate vesicular staining with zoom in inset showing vessicle staining.

FIGS. 2A and B—is an image showing specific detection of the ROS fusion/translocation (in a human NSCLC cell line) by FISH using a 2-color break-a-part probe. FIG. 2A shows the locations on the ROS gene where the FISH probes hybridize, and FIG. 2B shows the rearrangement of the ROS gene in a human NSCLC cell line (left) and a human NSCLC tumor, resulting in separate orange and green signals.

FIG. 3 is a schematic diagram showing where the DNA probes of the two probe sets hybridize to the ROS gene and the FIG gene. The proximal probe of both probe sets, namely RP1-179P9, will give an orange signal while all three distal probes will give a green signal. Probe set 1 was derived from c-ros, and if a balanced translocation occurs, the orange will separated from the green; however if a FIG-ROS translocation occurs, the green signal will disappear. Probe set 2 was derived from c-ros (orange RP1-179P9) and fig (green RP11-213A17).

FIG. 4 are photographs showing the results of FISH analysis of HCC78 cells, U118MG cells and FFPE tumor ID 749. HCC78 cells probed with probe set 1 and probe set 2 shows results expected from the SLC34A2-ROS fusion present in these cells. Yellow arrows point to split signals indicative of balanced translocation in HCC78 cells and white arrows point to intact chromosome. U118MG cells probed with probe set 1 and probe set 2 shows results expected from the FIG-ROS fusion present in these cells. FFPE tumor 749 probed with probe set 1 and probe set 2 is identical to U118MG cells. In both U-118 MG and Tumor ID 749 probed with probe set 1 only the c-ros (orange) probe anneals and the deleted region (green probe) is not present. In U-118 MG and Tumor ID 749 probed with probe set 2, the c-ros (orange) and fig (green) probes come together indicating a FIG-ROS fusion.

FIG. 5 shows the results of cDNA sequencing of the ROS fusion protein from tumor 749 (in “sbjt” line) and its alignment with the FIG-ROS(S) nucleotide sequence (as “query”).

FIG. 6 is a line graph showing the cellular growth response in the presence of 0 nM, 3 nM, 10 nM, 30 nM, 100 nM, 300 nM or 1000 nM TAE-684 of BaF3 expressing FIG-ROS(S) (red squares), BaF3 expressing FIG-ROS(L) (blue diamonds), BaF3 expressing FLT3ITD (green triangles), and Karpas 299 cells (purple Xs).

FIG. 7 is a bar graph showing that BaF3 expressing either FIG-ROS(S) or FIG-ROS(L) die by apoptosis in the presence of TAE-684.

FIG. 8 is a depiction of a Western blotting analysis showing that phosphorylation of both FIG-ROS(S) and FIG-ROS(L), as well as their downstream signaling molecules, are inhibited by TAE-684.

FIGS. 9A and 9B are line graphs showing the cellular growth response in the presence of TAE-684 (FIG. 9A) or crizotinib (FIG. 9B) at 0 uM, 0.01 uM, 0.03 M, 0.10 uM, 0.3 uM, 1.0 uM of BaF3 cells transduced with neo-myc (negative control; blue diamonds); BaF3 expressing FIG-ROS(S) (purple X's), BaF3 expressing FIG-ROS(L) (green triangles), BaF3 expressing FLT3ITD (red squares), and Karpas 299 cells (blue asterisks).

FIG. 10 is a depiction of a Western blotting analysis showing that phosphorylation of both FIG-ROS(S) and FIG-ROS(L), as well as ALK and additional signaling molecules are inhibited by crizotinib.

### DETAILED DESCRIPTION OF THE PREFERRED EMBODIMENTS

The invention is based upon the discovery that ROS and ALK are independent drivers of cancer. In other words, if ROS kinase is the driver of a cancer, ALK will not drive the cancer. Similarly, if ALK kinase is the driver of a cancer, ROS will not drive that cancer. This discovery will allow for identification of patients whose tumors will benefit from therapy with an ALK-inhibiting therapeutic or a ROS-inhibiting therapeutic, or both.

The published patents, patent applications, websites, company names, and scientific literature referred to herein establish the knowledge that is available to those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

The further aspects, advantages, and embodiments of the invention are described in more detail below. The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter. As used herein, the following terms have the meanings indicated. As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of antibody and recombinant DNA technology, all of which are incorporated herein by reference in their entirety, include Harlow and Lane, Antibodies, a Laboratory Manual, Cold Spring Harbor Laboratory Press, New York (1988), Ausubel et al. Current Protocols in Molecular Biology, John Wiley & Sons, New York, N.Y. (1989 and updates through September 2010), Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology, all of which are incorporated herein by reference in their entirety, include Goodman and Gilman's The Pharmacological Basis of Therapeutics, 11th Ed., McGraw Hill Companies Inc., New York (2006).

Accordingly, in a first aspect, the invention provides a method of treating a patient for cancer, comprising: detecting the presence in a biological sample from a patient having or suspected of having cancer of a polypeptide selected from the group consisting of a polypeptide having ROS kinase activity and a polypeptide having ALK kinase activity; and administering a therapeutically effective amount of an ALK/ROS-inhibiting therapeutic to the patient, thereby treating the patient for cancer. In some embodiments, the detecting step is performed by using a reagent that specifically binds to either a polypeptide having ROS kinase activity or a polypeptide having ALK kinase activity.

Human ROS kinase protein (encoded by the ROS1 gene) is a 2347 amino acid long receptor tyrosine kinase that is prone to aberrant expression leading to cancer. A description of full length human ROS kinase (with the amino acid sequence of the human ROS protein) can be found at UniProt Accession No. P08922. As shown in Table 1, the signal peptide, extracellular, transmembrane, and kinase domains of ROS are found at the following amino acid residues in SEQ ID NO: 1:

TABLE 1

| Domain | Amino acid residues in SEQ ID NO: 1 |
|---|---|
| Signal peptide | 1-27 |
| Extracellular domain | 28-1859 |
| Transmembrane domain | 1860-1882 |
| Kinase domain | 1945-2222 |

The coding DNA sequence of human ROS is provided herein as SEQ ID NO: 2.

Additionally, there are multiple known naturally-occurring variants of ROS (see, e.g., Greenman et al., Nature 446: 153-158, 2007). The nucleotide and amino acid sequences of murine full-length ROS are known (see, e.g., UniProt Accession No. Q78DX7). Using routine experimentation, the ordinarily skilled biologist would be readily able to determine corresponding sequences in non-human mammalian ROS homologues.

By “wild-type” ROS is meant the expression and/or activation of full length ROS kinase (i.e., for human ROS, the 2347 amino acid long polypeptide or 2320 amino acid long polypeptide following removal of the signal peptide sequence) in healthy (or normal) tissue (e.g., non-cancerous tissue) of a normal individual (e.g., a normal individual who is not suffering from cancer). ROS kinase (full length or truncated) does not appear to be expressed in normal lung tissue in humans (e.g., see below in the Examples). However, using the methods described in the below Examples, the inventors have made the surprising discovery of ROS kinase expression in lung cancer. Such expression in an atypical cell (in this case a cancerous cell) where no expression is seen in a typical cell (e.g., a non-cancerous lung cell) is aberrant.

Aberrantly expressed ROS kinase, in the form of a fusion with another protein, namely FIG, has been reported in glioblastoma (see Charest et al., Charest et al., Genes Chromosomes Cancer 37: 58-71, 2003; Charest et al., Proc. Natl. Acad. Sci. USA 100: 916-921, 2003) and in liver cancer (see, e.g., PCT Publication No. WO2010/093928).

As used herein, the term “ROS fusion” refers to a portion of the ROS polypeptide comprising the kinase domain of the ROS protein (or polynucleotide encoding the same) fused to all or a portion of another polypeptide (or polynucleotide encoding the same), where the name of that second polypeptide or polynucleotide is named in the fusion. (The term “fusion” simply means all or a portion of a polypeptide or polynucleotide from first gene fused to all or a portion of a polypeptide or a polynucleotide from a second gene). For example, an SLC34A2-ROS fusion is a fusion between a portion of the SLC34A2 polypeptide (or polynucleotide encoding the same) and a portion of the ROS polypeptide (or polynucleotide encoding the same) comprising the kinase domain ROS. An ROS fusion often results from a chromosomal translocation or inversion. There are numerous known ROS fusions, all of which are ROS fusions of the invention and include, without limitation, the SLC34A2-ROS fusion proteins whose members include SLC34A2-ROS(VS), SLC34A2-ROS(S), SLC34A2-ROS(L) (see U.S. Patent Publication No. 20100143918), CD74-ROS (see U.S. Patent Publication No. 20100221737) and the FIG-ROS fusion proteins whose members include FIG-ROS(S), FIG-ROS(L), and FIG-ROS(XL) (see PCT Publication No. WO2010/093928).

All of the known ROS fusion proteins comprise the full kinase domain of full length ROS. Thus, as used herein, by a “polypeptide with ROS kinase activity” (or “polypeptide having ROS kinase activity”) is meant a protein (or polypeptide) that includes the full kinase domain of full length ROS protein and, thus, retains ROS kinase activity. Non-limiting examples of proteins with ROS kinase activity include, without limitation, full length ROS protein, the SLC34A2-ROS fusion proteins, whose members include SLC34A2-ROS(VS), SLC34A2-ROS(S), SLC34A2-ROS(L) (see U.S. Patent Publication No. 20100143918), CD74-ROS (see U.S. Patent Publication No. 20100221737) and the FIG-ROS fusion proteins whose members include FIG-ROS(S), FIG-ROS(L), and FIG-ROS(XL) (see PCT Publication No. WO2010/093928), and any truncated or mutated form of ROS kinase that retains the kinase domain of full-length ROS kinase protein. As the kinase domain of ROS is set forth in SEQ ID NO: 24, a “polypeptide with ROS kinase activity” is one whose amino acid sequence comprises SEQ ID NO: 24.

ALK (anaplastic lymphoma kinase) is a 1620 amino acid long receptor tyrosine kinase that is prone to aberrant expression leading to cancer. A description of full length human ALK kinase (with the amino acid sequence of the human ALK protein) can be found at UniProt Accession No. Q9UM73 (see also U.S. Pat. No. 5,770,421, entitled “Human ALK Protein Tyrosine Kinase”). As shown in Table 2, the signal peptide, extracellular, transmembrane, and kinase domains of ALK are found at the following amino acid residues in SEQ ID NO: 32:

TABLE 2

| Domain | Amino acid residues in SEQ ID NO: 32 |
|---|---|
| Signal peptide | 1-18 |
| Extracellular domain | 19-1038 |
| Transmembrane domain | 1039-1059 |
| Kinase domain | 1116-1392 |

Additionally, there are multiple known naturally-occurring variants of ALK (see, e.g., Greenman et al., Nature 446: 153-158, 2007). The nucleotide and amino acid sequences of murine full-length ALK are known (see lawahara et al., Oncogene 14(4): 439-449, 1997). Using routine experimentation, the ordinarily skilled biologist would be readily able to determine corresponding sequences in non-human mammalian ALK homologues.

By “wild-type” ALK is meant the expression and/or activation of full length ALK kinase (i.e., 1620 amino acid long polypeptide or 1602 amino acid long polypeptide following removal of the signal peptide sequence) in healthy (or normal) tissue (e.g., non-cancerous tissue) of a normal individual (e.g., a normal individual who is not suffering from cancer). Pulford et al., Journal of Cellular Physiology, 199:330-358, 2004 provides a comprehensive review relating to ALK and fusion polypeptides that include portions of the full length ALK polyepeptide. In normal humans, full-length ALK expression has been detected in the brain and central nervous system, and has been reported in the small intestine and testis (see, e.g., Morris et al., Oncogene 14:2175-2188, 1997). However, ALK kinase (full length or truncated) does not appear to be expressed in normal ovarian tissue in humans, a finding which the inventors have confirmed using various commercially available ALK-specific antibodies (e.g., Catalog Nos. 3791 and 3333 from Cell Signaling Technology, Inc., Danvers, Mass.). However, using the methods described in the below Examples, the inventors have made the surprising discovery of ALK kinase expression in ovarian cancer. Such expression in an atypical cell (in this case a cancerous cell) where no expression is seen in a typical cell (e.g., a non-cancerous ovarian cell) is aberrant.

Numerous examples of aberrantly expressed ALK kinase have been found in other cancers. For example, point mutations within the kinase domain have been found in neuroblastoma, overexpression of ALK has been found in numerous cancers (including, e.g., retinoblastoma, breast cancer, and melanoma), and fusion proteins comprising the kinase domain (but not the transmembrane domain) of ALK fused to all or a portion of a second protein have been discovered in various cancers including non-small cell lung cancer (NSCLC) in inflammatory myofibroblastic tumor. See review in Palmer et al., Biochem. J. 420(3): 345-361 (May 2009), herein incorporated by reference in its entirety.

Accordingly, as used herein, the term “ALK fusion” refers to a portion of the ALK polypeptide comprising the kinase domain of ALK (polynucleotide encoding the same) fused to all or a portion of another polypeptide (or polynucleotide encoding the same), where the name of that second polypeptide or polynucleotide is named in the fusion. (The term “fusion” simply means all or a portion of a polypeptide or polynucleotide from first gene fused to all or a portion of a polypeptide or a polynucleotide from a second gene). For example, an NPM-ALK fusion is a fusion between a portion of the NPM polypeptide or polynucleotide and a portion of the ALK polypeptide (or polynucleotide encoding the same) comprising the kinase domain of ALK. An ALK fusion often results from a chromosomal translocation or inversion. There are numerous known ALK fusions, all of which are ALK fusions of the invention and include, without limitation, NPM-ALK, ALO17-ALK, TFG-ALK, MSN-ALK, TPM3-ALK, TPM4-ALK, ATIC-ALK, MYH9-ALK, CLTC-ALK, SEC31L1-ALK, RANBP2-ALK, CARS-ALK, EML4-ALK, KIF5B-ALK, and TFG-ALK (see, e.g., Palmer et al., Biochem. J. 420(3): 345-361, 2009 (and the articles cited therein), U.S. Pat. No. 5,770,421; Rikova et al., Cell 131: 1190-1203, 2007; Soda et al., Nature 448: 561-566, 2007; Morris et al., Science 263: 1281-1284, 1994; Du et al., J. Mol. Med 84: 863-875, 2007; Panagopoulos et al., Int. J. Cancer 118: 1181-1186, 2006; Cools et al., Genes Chromosomes Cancer 34: 354-362, 2002; Debelenko et al., Lab. Invest. 83: 1255-1265, 2003; Ma et al., Genes Chromosomes Cancer 37: 98-105, 2003; Lawrence et al., Am. J. Pathol. 157: 377-384, 1995; Hernandez et al., Blood 94: 3265-3268, 1999; Takeuchi K., Clin Cancer Res. 15(9):3143-3149, 2009; Tort et al., Lab. Invest. 81: 419-426, 2001; Trinei et al., Cancer Res. 60: 793-798, 2000; and Touriol et al., Blood 95: 3204-3207, 2000, all hereby incorporated by reference in their entirety. Some of these ALK fusions have multiple variants, all of which are considered ALK fusions and, thus, are included in the definition of ALK fusion of the invention. For example, there are multiple variants of TFG-ALK (see, e.g., Hernandez et al., Amer. J. Pathol. 160: 1487-1494, 2002) and at least nine known variants of EML4-ALK (see, e.g., Horn et al., J. of Clinical Oncology 27(26): 4232-4235, 2009, U.S. Pat. Nos. 7,700,339 and 7,728,120 and EP Patent No. 1 914 240, all hereby incorporated by reference in their entirety).

As used herein, by the term “polypeptide with ALK kinase activity” is meant any polypeptide that retains the full kinase domain of ALK and thus, has ALK kinase activity. Non-limiting polypeptides with ALK kinase activity include full length ALK, ALK fusion polypeptides (e.g., NPM-ALK fusion, various EML4-ALK fusions, ATIC-ALK fusion, CARS-ALK fusion, ALO17-ALK fusion, TFG-ALK fusion, MSN-ALK fusion, TPM3-ALK fusion, TPM4-ALK fusion, MYH9-ALK fusion, CLTC-ALK fusion, SEC31L1-ALK fusion, RANBP2-ALK fusion, KIF5B-ALK fusion, and TFG-ALK fusion).

As used herein, by “polypeptide” (or “amino acid sequence” or “protein”) refers to a polymer formed from the linking, in a defined order, of preferably, a-amino acids, D-, L-amino acids, and combinations thereof. The link between one amino acid residue and the next is referred to as an amide bond or a peptide bond. Non-limiting examples of polypeptides include refers to an oligopeptide, peptide, polypeptide, or protein sequence, and fragments or portions thereof, and to naturally occurring or synthetic molecules. Polypeptides also include derivatized molecules such as glycoproteins and lipoproteins as well as lower molecular weight polypeptides. “Amino acid sequence” and like terms, such as “polypeptide” or “protein”, are not meant to limit the indicated amino acid sequence to the complete, native amino acid sequence associated with the recited protein molecule.

It will be recognized in the art that some amino acid sequences of an indicated polypeptide (e.g., a FIG-ROS(S) polypeptide) can be varied without significant effect of the structure or function of the mutant protein. If such differences in sequence are contemplated, it should be remembered that there will be critical areas on the protein which determine activity (e.g. the kinase domain of ROS). In general, it is possible to replace residues that form the tertiary structure, provided that residues performing a similar function are used. In other instances, the type of residue may be completely unimportant if the alteration occurs at a non-critical region of the protein.

Thus, a polypeptide with ROS activity or a polypeptide with ALK activity further includes variants of the polypeptides described herein that shows substantial ROS kinase activity or ALK knase activity. Some non-limiting conservative substitutions include the exchange, one for another, among the aliphatic amino acids Ala, Val, Leu and Ile; exchange of the hydroxyl residues Ser and Thr; exchange of the acidic residues Asp and Glu; exchange of the amide residues Asn and Gln; exchange of the basic residues Lys and Arg; and exchange of the aromatic residues Phe and Tyr. Further examples of conservative amino acid substitutions known to those skilled in the art are: Aromatic: phenylalanine tryptophan tyrosine (e.g., a tryptophan residue is replaced with a phenylalanine); Hydrophobic: leucine isoleucine valine; Polar: glutamine asparagines; Basic: arginine lysine histidine; Acidic: aspartic acid glutamic acid; Small: alanine serine threonine methionine glycine. As indicated in detail above, further guidance concerning which amino acid changes are likely to be phenotypically silent (i.e., are not likely to have a significant deleterious effect on a function) can be found in Bowie et al., Science 247, supra.

In some embodiments, a variant may have “nonconservative” changes, e.g., replacement of a glycine with a tryptophan. Similar variants may also include amino acid deletions or insertions, or both. Guidance in determining which amino acid residues may be substituted, inserted, or deleted without abolishing biological or immunological activity may be found using computer programs well known in the art, for example, DNASTAR software.

The polypeptides having ROS kinase activity of the present invention include the full length human ROS protein (having an amino acid sequence set forth in SEQ ID NO: 1) and the ROS fusion polypeptides having the amino sequences set forth in SEQ ID NOs: 3, 5, 8, 10, 19, 21, and 23 (whether or not including a leader sequence), an amino acid sequence encoding a polypeptide comprising at least six contiguous amino acids encompassing the fusion junction (i.e., the sequences at the junction between the non-ROS partner protein and the ROS protein; see Table 3, as well as polypeptides that have at least 90% similarity, more preferably at least 95% similarity, and still more preferably at least 96%, 97%, 98% or 99% similarity to those described above.

The polypeptides having ALK kinase activity of the present invention include the full length human ALK protein (having an amino acid sequence set forth in SEQ ID NO: 32) and the various ALK fusion polypeptides described herein, an amino acid sequence encoding a polypeptide comprising at least six contiguous amino acids encompassing the fusion junction (i.e., the sequences at the junction between the non-ALK partner protein and the ALK protein, as well as polypeptides that have at least 90% similarity, more preferably at least 95% similarity, and still more preferably at least 96%, 97%, 98% or 99% similarity to those described above.

Full length ROS-specific reagents and the ROS fusion polypeptide specific reagents (such as polyclonal and monoclonal antibodies) or full length ALK-specific reagents and the ALK fusion polypeptide specific reagents (such as polyclonal and monoclonal antibodies) which are useful in assays for detecting ROS or ALK polypeptide expression and/or ROS or ALK kinase activity as described below or as ROS-inhibiting therapeutics or ALK-inhibiting capable of inhibiting ROS protein function/activity and/or ALK protein function/activity. Further, such polypeptides can be used in the yeast two-hybrid system to “capture” binding proteins, which are also candidate ROS-inhibiting therapeutics or ALK-inhibiting therapeutics according to the present invention. The yeast two hybrid system is described in Fields and Song, Nature 340: 245-246 (1989).

In some embodiments, the reagent may further comprise a detectable label (e.g., a fluorescent label or an infrared label). By “detectable label” with respect to a polypeptide, polynucleotide, or reagent (e.g., antibody or FISH probe) disclosed herein means a chemical, biological, or other modification of or to the polypeptide, polynucleotide, or antibody, including but not limited to fluorescence (e.g., FITC or phycoerythrin), infrared, mass (e.g., an isobaric tag), residue, dye (chromophoric dye), radioisotope (e.g., 32P), label, or tag (myc tag or GST tag) modifications, etc., by which the presence of the molecule of interest may be detected. Such a polypeptide, polynucleotide, or reagent thus called “detectably labeled.” The detectable label may be attached to the polypeptide, polynucleotide, or binding agent by a covalent (e.g., peptide bond or phosphodiester bond) or non-covalent chemical bond (e.g., an ionic bond).

Reagents useful in the methods of the invention include, without limitation, reagents such as antibodies or AQUA peptides, or binding fractions thereof, that specifically bind to full length ROS protein or one of the many ROS fusion proteins, or to to full length ROS protein or one of the many ROS fusion proteins expressed in cancer. By “specifically binding” or “specifically binds” means that a reagent or binding agent of the invention (e.g., a nucleic acid probe, an antibody, or AQUA peptide) interacts with its target molecule where the interaction is dependent upon the presence of a particular structure (e.g., the antigenic determinant or epitope on the polypeptide or the nucleotide sequence of the polynucleotide); in other words, the reagent is recognizing and binding to a specific polypeptide or polynucleotide structure rather than to all polypeptides or polynucleotides in general. By “binding fragment thereof” means a fragment or portion of a reagent that specifically binds the target molecule (e.g., an Fab fragment of an antibody).

A reagent that specifically binds to the target molecule may be referred to as a target-specific reagent or an anti-target reagent. For example, an antibody that specifically binds to a FIG-ROS(L) polypeptide may be referred to as a FIG-ROS(L)-specific antibody or an anti-FIG-ROS(L) antibody. Similarly, a nucleic acid probe that specifically binds to a FIG-ROS(L) polynucleotide may be referred to as a FIG-ROS(L)-specific nucleic acid probe or an anti-FIG-ROS(L) nucleic acid probe.

In some embodiments, where the target molecule is a polypeptide, a reagent that specifically binds a target molecule has a binding affinity (K_{D}) for its target molecule of 1×10⁻⁶ M or less. In some embodiments, a reagent of the invention that specifically binds to a target molecule has for its target molecule a K_{D} of 1×10⁻⁷ M or less, or a K_{D} of 1×10⁻⁸ M or less, or a K_{D} of 1×10⁻⁹ M or less, or a K_{D} of 1×10⁻¹⁰ M or less, of a K_{D} of 1×10⁻¹¹ M or less, of a K_{D} of 1×10⁻¹² M or less. In certain embodiments, the K_{D} of a reagent of the invention that specifically binds to a target molecule is 1 pM to 500 pM, or between 500 pM to 1 μM, or between 1 μM to 100 nM, or between 100 mM to 10 nM for its target molecule. Non-limiting examples of a target molecule to which a reagent of the invention specifically binds to include full length ROS polypeptide, the full length ALK polypeptide, or one of the many ALK fusion proteins and/or the ROS fusion polypeptides described herein.

In some embodiments, where the target molecule is a polynucleotide, a reagent of the invention that specifically binds its target molecule is a reagent that hybridizes under stringent conditions to it target polynucleotide. The term “stringent conditions” with respect to nucleotide sequence or nucleotide probe hybridization conditions is the “stringency” that occurs within a range from about Tₘ minus 5° C. (i.e., 5° C. below the melting temperature (Tₘ) of the reagent or nucleic acid probe) to about 20° C. to 25° C. below Tₘ. Typical stringent conditions are: overnight incubation at 42° C. in a solution comprising: 50% formamide, 5×.SSC (750 mM NaCl, 75 mM trisodium citrate), 50 mM sodium phosphate (pH 7.6), 5×Denhardt's solution, 10% dextran sulfate, and 20 micrograms/ml denatured, sheared salmon sperm DNA, followed by washing the filters in 0.1×SSC at about 65° C. As will be understood by those of skill in the art, the stringency of hybridization may be altered in order to identify or detect identical or related polynucleotide sequences. By a “reagent (e.g., a polynucleotide or nucleotide probe) that hybridizes under stringent conditions to a target polynucleotide (e.g., a full length ROS polynucleotide)” is intended that the reagent (e.g., the polynucleotide or nucleotide probe (e.g., DNA, RNA, or a DNA-RNA hybrid)) hybridizes along the entire length of the reference polynucleotide or hybridizes to a portion of the reference polynucleotide that is at least about 15 nucleotides (nt), or to at least about 20 nt, or to at least about 30 nt, or to about 30-70 nt of the reference polynucleotide. These nucleotide probes of the invention are useful as diagnostic probes (e.g., for FISH) and primers (e.g., for PCR) as discussed herein.

The reagents useful in the practice of the disclosed methods, include, among others, full length ROS-specific and ROS fusion polypeptide-specific antibodies, full length ALK-specific and ALK fusion polypeptide-specific antibodies, and AQUA peptides (heavy-isotope labeled peptides) corresponding to, and suitable for detection and quantification of, the indicated polypeptide's expression in a biological sample. Thus, a “ROS polypeptide-specific reagent” is any reagent, biological or chemical, capable of specifically binding to, detecting and/or quantifying the presence/level of expressed ROS polypeptide in a biological sample. Likewise, an “ALK polypeptide-specific reagent” is any reagent, biological or chemical, capable of specifically binding to, detecting and/or quantifying the presence/level of expressed ALK polypeptide in a biological sample. The terms include, but are not limited to, the antibodies and AQUA peptide reagents discussed below, and equivalent binding agents are within the scope of the present invention.

The antibodies that specifically binds to full length ROS porotein, to one of the ROS fusion polypeptides, to full length ALK protein, or to one of the ALK fusion polypeptides in cancer may also bind to highly homologous and equivalent epitopic peptide sequences in other mammalian species, for example murine or rabbit, and vice versa. Antibodies useful in practicing the methods of the invention include (a) monoclonal antibodies, (b) purified polyclonal antibodies that specifically bind to the target polypeptide (e.g., the fusion junction of the fusion polypeptide, (c) antibodies as described in (a)-(b) above that specifically bind equivalent and highly homologous epitopes or phosphorylation sites in other non-human species (e.g., mouse, rat), and (d) fragments of (a)-(c) above that specifically bind to the antigen (or more preferably the epitope) bound by the exemplary antibodies disclosed herein.

The term “antibody” or “antibodies” refers to all types of immunoglobulins, including IgG, IgM, IgA, IgD, and IgE, including binding fragments thereof (i.e., fragments of an antibody that are capable of specifically binding to the antibody's target molecule, such as F_{ab}, and F(ab′)₂ fragments), as well as recombinant, humanized, polyclonal, and monoclonal antibodies and/or binding fragments thereof. Antibodies of the invention can be derived from any species of animal, such as from a mammal. Non-limiting exemplary natural antibodies include antibodies derived from human, chicken, goats, and rodents (e.g., rats, mice, hamsters and rabbits), including transgenic rodents genetically engineered to produce human antibodies (see, e.g., Lonberg et al., WO93/12227; U.S. Pat. No. 5,545,806; and Kucherlapati, et al., WO91/10741; U.S. Pat. No. 6,150,584, which are herein incorporated by reference in their entirety). Antibodies of the invention may be also be chimeric antibodies. See, e.g., M. Wroser et al., Molec. Immunol. 26: 403-11 (1989); Morrision et al., Proc. Nat'l. Acad. Sci. 81: 6851 (1984); Neuberger et al., Nature 312: 604 (1984)). The antibodies may be recombinant monoclonal antibodies produced according to the methods disclosed in U.S. Pat. No. 4,474,893 (Reading) or U.S. Pat. No. 4,816,567 (Cabilly et al.) The antibodies may also be chemically constructed specific antibodies made according to the method disclosed in U.S. Pat. No. 4,676,980 (Segel et al.).

Natural antibodies are the antibodies produced by a host animal, however the invention contemplates also genetically altered antibodies wherein the amino acid sequence has been varied from that of a native antibody. Because of the relevance of recombinant DNA techniques to this application, one need not be confined to the sequences of amino acids found in natural antibodies; antibodies can be redesigned to obtain desired characteristics. The possible variations are many and range from the changing of just one or a few amino acids to the complete redesign of, for example, the variable or constant region. Changes in the constant region will, in general, be made in order to improve or alter characteristics, such as complement fixation, interaction with membranes and other effector functions. Changes in the variable region will be made in order to improve the antigen binding characteristics. The term “humanized antibody”, as used herein, refers to antibody molecules in which amino acids have been replaced in the non-antigen binding regions in order to more closely resemble a human antibody, while still retaining the original binding ability. Other antibodies specifically contemplated are oligoclonal antibodies. As used herein, the phrase “oligoclonal antibodies” refers to a predetermined mixture of distinct monoclonal antibodies. See, e.g., PCT publication WO 95/20401; U.S. Pat. Nos. 5,789,208 and 6,335,163. In one embodiment, oligoclonal antibodies consisting of a predetermined mixture of antibodies against one or more epitopes are generated in a single cell. In other embodiments, oligoclonal antibodies comprise a plurality of heavy chains capable of pairing with a common light chain to generate antibodies with multiple specificities (e.g., PCT publication WO 04/009618). Oligoclonal antibodies are particularly useful when it is desired to target multiple epitopes on a single target molecule. In view of the assays and epitopes disclosed herein, those skilled in the art can generate or select antibodies or mixtures of antibodies that are applicable for an intended purpose and desired need.

Recombinant antibodies are also included in the present invention. These recombinant antibodies have the same amino acid sequence as the natural antibodies or have altered amino acid sequences of the natural antibodies. They can be made in any expression systems including both prokaryotic and eukaryotic expression systems or using phage display methods (see, e.g., Dower et al., WO91/17271 and McCafferty et al., WO92/01047; U.S. Pat. No. 5,969,108, which are herein incorporated by reference in their entirety). Antibodies can be engineered in numerous ways. They can be made as single-chain antibodies (including small modular immunopharmaceuticals or SMIPs™), Fab and F(ab′)₂ fragments, etc. Antibodies can be humanized, chimerized, deimmunized, or fully human. Numerous publications set forth the many types of antibodies and the methods of engineering such antibodies. For example, see U.S. Pat. Nos. 6,355,245; 6,180,370; 5,693,762; 6,407,213; 6,548,640; 5,565,332; 5,225,539; 6,103,889; and 5,260,203. The genetically altered antibodies of the invention may be functionally equivalent to the above-mentioned natural antibodies. In certain embodiments, modified antibodies of the invention provide improved stability or/and therapeutic efficacy.

Non-limiting examples of modified antibodies include those with conservative substitutions of amino acid residues, and one or more deletions or additions of amino acids that do not significantly deleteriously alter the antigen binding utility. Substitutions can range from changing or modifying one or more amino acid residues to complete redesign of a region as long as the therapeutic utility is maintained. Antibodies of the invention can be modified post-translationally (e.g., acetylation, and/or phosphorylation) or can be modified synthetically (e.g., the attachment of a labeling group). Antibodies with engineered or variant constant or Fc regions can be useful in modulating effector functions, such as, for example, antigen-dependent cytotoxicity (ADCC) and complement-dependent cytotoxicity (CDC). Such antibodies with engineered or variant constant or Fc regions may be useful in instances where a parent singling protein is expressed in normal tissue; variant antibodies without effector function in these instances may elicit the desired therapeutic response while not damaging normal tissue. Accordingly, certain aspects and methods of the present disclosure relate to antibodies with altered effector functions that comprise one or more amino acid substitutions, insertions, and/or deletions. The term “biologically active” refers to a protein having structural, regulatory, or biochemical functions of a naturally occurring molecule. Likewise, “immunologically active” refers to the capability of the natural, recombinant, or synthetic polypeptide (e.g., one of the ROS or ALK fusion polypeptides described herein), or any oligopeptide thereof, to induce a specific immune response in appropriate animals or cells and to bind with specific antibodies.

Also within the invention are antibody molecules with fewer than 4 chains, including single chain antibodies, Camelid antibodies and the like and components of an antibody, including a heavy chain or a light chain. In some embodiments an immunoglobulin chain may comprise in order from 5′ to 3′, a variable region and a constant region. The variable region may comprise three complementarity determining regions (CDRs), with interspersed framework (FR) regions for a structure FR1, CDR1, FR2, CDR2, FR3, CDR3 and FR4. Also within the invention are heavy or light chain variable regions, framework regions and CDRs. An antibody of the invention may comprise a heavy chain constant region that comprises some or all of a CH1 region, hinge, CH2 and CH3 region.

One non-limiting epitopic site of a fusion polypeptide-specific antibody of the invention is a peptide fragment consisting essentially of about 11 to 17 amino acids of a fusion polypeptide sequence, which fragment encompasses the fusion junction between the ROS portion of the molecule and the portion of the molecule from the non-ROS fusion partner. It will be appreciated that antibodies that specifically binding shorter or longer peptides/epitopes encompassing the fusion junction of a ROS fusion polypeptide are within the scope of the present invention.

The invention is not limited to use of antibodies, but includes equivalent molecules, such as protein binding domains or nucleic acid aptamers, which bind, in a ROS proten-speicific or ROS fusion protein-specific manner, to essentially the same epitope to which a full length ROS-specific or ROS fusion polpeptide-specific antibody useful in the methods of the invention binds. See, e.g., Neuberger et al., Nature 312: 604 (1984). Such equivalent non-antibody reagents may be suitably employed in the methods of the invention further described below.

Polyclonal antibodies useful in practicing the methods of the invention may be produced according to standard techniques by immunizing a suitable animal (e.g., rabbit, goat, etc.) with an antigen encompassing a desired fusion-protein specific epitope (e.g. the fusion junction between the non-ROS protein partner and the ROS protein partner in a ROS fusion polypeptide), collecting immune serum from the animal, and separating the polyclonal antibodies from the immune serum, and purifying polyclonal antibodies having the desired specificity, in accordance with known procedures. The antigen may be a synthetic peptide antigen comprising the desired epitopic sequence, selected and constructed in accordance with well-known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85: 21-49 (1962)). Polyclonal antibodies produced as described herein may be screened and isolated as further described below.

Monoclonal antibodies may also be beneficially employed in the methods of the invention, and may be produced in hybridoma cell lines according to the well-known technique of Kohler and Milstein. Nature 265: 495-97 (1975); Kohler and Milstein, Eur. J. Immunol. 6: 511 (1976); see also, CURRENT PROTOCOLS IN MOLECULAR BIOLOGY, Ausubel et al. Eds. (Wiley and Sins, New York, N.Y. 1989 and yearly updates up to and including 2010). Monoclonal antibodies so produced are highly specific, and improve the selectivity and specificity of assay methods provided by the invention. For example, a solution containing the appropriate antigen (e.g. a synthetic peptide comprising the fusion junction of ROS fusion polypeptide) may be injected into a mouse and, after a sufficient time (in keeping with conventional techniques), the mouse sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art. See, e.g., W. Huse, Science 246: 1275-81 (1989); Mullinax et al., Proc. Nat'l Acad. Sci. 87: 8095 (1990). If monoclonal antibodies of one isotype are desired for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)). The antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

Further still, U.S. Pat. No. 5,194,392, Geysen (1990) describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, this method involves detecting or determining a sequence of monomers which is a topographical equivalent of a ligand which is complementary to the ligand binding site of a particular receptor of interest. Similarly, U.S. Pat. No. 5,480,971, Houghten et al. (1996) discloses linear C₁-C-rosyl perrosylated oligopeptides and sets and libraries of such peptides, as well as methods for using such oligopeptide sets and libraries for determining the sequence of a perrosylated oligopeptide that preferentially binds to an acceptor molecule of interest. Thus, non-peptide analogs of the epitope-bearing peptides of the invention also can be made routinely by these methods.

Antibodies useful in the methods of the invention, whether polyclonal or monoclonal, may be screened for epitope and fusion protein specificity according to standard techniques. See, e.g., Czernik et al., Methods in Enzymology, 201: 264-283 (1991). For example, the antibodies may be screened against a peptide library by ELISA to ensure specificity for both the desired antigen and, if desired, for reactivity only with the full-length ROS protein, a particular ROS fusion polypeptide (e.g., an SLC34A2-ROS(S) polypeptide), or fragments thereof of the invention. The antibodies may also be tested by Western blotting against cell preparations containing target protein to confirm reactivity with the only the desired target and to ensure no appreciable binding to other proteins. The production, screening, and use of fusion protein-specific antibodies is known to those of skill in the art, and has been described. See, e.g., U.S. Patent Publication No. 20050214301.

Antibodies useful in the methods of the invention may exhibit some limited cross-reactivity with similar epitopes in other proteins or polypeptides, such as similar fusion polypeptides. This is not unexpected as most antibodies exhibit some degree of cross-reactivity, and anti-peptide antibodies will often cross-react with epitopes having high homology or identity to the immunizing peptide. See, e.g., Czernik, supra. Cross-reactivity with other fusion proteins is readily characterized by Western blotting alongside markers of known molecular weight. Undesirable cross-reactivity can be removed by negative selection using antibody purification on peptide columns.

ROS-specific antibodies and ROS fusion polypeptide-specific antibodies of the invention that are useful in practicing the methods disclosed herein are ideally specific for human fusion polypeptide, but are not limited only to binding the human species, per se. The invention includes the production and use of antibodies that also bind conserved and highly homologous or identical epitopes in other mammalian species (e.g., mouse, rat, monkey). Highly homologous or identical sequences in other species can readily be identified by standard sequence comparisons, such as using BLAST, with the human ROS protein sequence (SEQ ID NO: 1), and the human ROS fusion polypeptide sequences disclosed herein (SEQ ID NOs: 3, 5, 8, 10, 19, 21, and 23).

ALK-specific antibodies and ALK fusion polypeptide-specific antibodies of the invention that are useful in practicing the methods disclosed herein are ideally specific for human fusion polypeptide, but are not limited only to binding the human species, per se. The invention includes the production and use of antibodies that also bind conserved and highly homologous or identical epitopes in other mammalian species (e.g., mouse, rat, monkey). Highly homologous or identical sequences in other species can readily be identified by standard sequence comparisons, such as using BLAST, with the human ALK protein sequence (SEQ ID NO: 32), and the human ALK fusion polypeptide sequences previously described.

Antibodies employed in the methods of the invention may be further characterized by, and validated for, use in a particular assay format, for example FC, IHC, and/or ICC. The use of full-length ROS protein-specific and/or ROS fusion polypeptide-specific antibodies and/or full-length ALK protein-specific and/or ALK fusion polypeptide-specific antibodies in such methods is further described herein. The antibodies described herein, used alone or in the below-described assays, may also be advantageously conjugated to fluorescent dyes (e.g. Alexa488, phycoerythrin), or labels such as quantum dots, for use in multi-parametric analyses along with other signal transduction (phospho-AKT, phospho-Erk 1/2) and/or cell marker (cytokeratin) antibodies, as further described below.

In practicing the methods of the invention, the expression and/or activity of a ROS fusion polypeptide of the invention and/or of full-length ROS in a given biological sample may also be advantageously examined using antibodies specific for (i.e., that specifically bind to) full length ROS protein or antibodies specific for ROS fusion polypeptides. For example, ROS-specific antibodies (i.e., antibodies that specifically bind full-length ROS) are commercially available (see Santa Cruz Biotech., Inc. (Santa Cruz, Calif.) Catalog No. sc-6347; Cell Signaling Technology, Inc. (Danvers, Mass.), Catalog No. 3266); and Abcam (Cambridge, Mass.), Catalog Nos. ab5512 and ab108492, for example). In some embodiments, ROS-specific antibodies used in the methods of the invention specifically bind the kinase domain of ROS and, thus, will detect full-length ROS and all of the ROS fusion polypeptides described herein. In some embodiments, ROS-specific antibodies used in the methods of the invention specifically bind a region on the ROS protein that is C'terminal to the kinase domain of ROS and, thus, will detect full-length ROS and all of the ROS fusion polypeptides described herein. Such antibodies may also be produced according to standard methods.

Likewise, the expression and/or activity of an ALK fusion polypeptide and/or of full-length ALK in a given biological sample may also be advantageously examined using antibodies specific for (i.e., that specifically bind to) full length ALK protein or antibodies specific for ALK fusion polypeptides. For example, ALK-specific antibodies (i.e., antibodies that specifically bind full-length ALK) are commercially available (see CELL SIGNALING TECHNOLOGY, INC., Danvers, Mass., Catalog Nos. 3333 and 3791; Abcam, 2010 Catalogue, #ab17127, ab59286, and Sigma-Aldrich, 2010 Catalog, #HPA010694, for example). In some embodiments, ALK-specific antibodies used in the methods of the invention specifically bind the kinase domain of ALK and, thus, will detect full-length ALK and the ALK fusion polypeptides described herein. Furthermore, ALK fusion-specific antibodies are commercially available (see CELL SIGNALING TECHNOLOGY, INC., Beverly Mass., 2009/10 Catalogue, #'s 3343S (phospho-NPM-ALK), 3983 (phospho-NPM-ALK), Abcam, 2010 Catalogue, #ab4061 (NPM-ALK), and Thermo Scientific, 2010 Catalogue, #PA1-37060 (NPM-ALK), for example). Such antibodies may also be produced according to standard methods, as described above.

Detection of expression and/or activity of full-length ROS and/or a ROS fusion polypeptide expression or full-length ALK and/or a ALK fusion polypeptide expression, in a biological sample (e.g. a tumor sample) can provide information on whether the kinase protein alone is driving the tumor, or whether aberrantly expressed full length ROS or ALK is also present and driving the tumor. Such information is clinically useful in assessing whether targeting the fusion protein or the full-length protein(s), or both, or is likely to be most beneficial in inhibiting progression of the tumor, and in selecting an appropriate therapeutic or combination thereof.

In some embodiments, a reagent that specifically binds to full length ROS or a ROS fusion polypeptide or full length ALK or an ALK fusion polypeptide is a heavy-isotope labeled peptide (i.e., an AQUA peptide) that, for example, corresponds to a peptide sequence comprising the fusion junction of a ROS or an ALK fusion polypeptide. Such an AQUA peptide may be suitable for the absolute quantification of an expressed ROS or ALK fusion polypeptide in a biological sample. As used herein, the term “heavy-isotope labeled peptide” is used interchangeably with “AQUA peptide”. The production and use of AQUA peptides for the absolute quantification or detection of proteins (AQUA) in complex mixtures has been described. See WO/03016861, “Absolute Quantification of Proteins and Modified Forms Thereof by Multistage Mass Spectrometry,” Gygi et al. and also Gerber et al., Proc. Natl. Acad. Sci. U.S.A. 100: 6940-5 (2003) (the teachings of which are hereby incorporated herein by reference, in their entirety). The term “specifically detects” with respect to such an AQUA peptide means the peptide will only detect and quantify polypeptides and proteins that contain the AQUA peptide sequence and will not substantially detect polypeptides and proteins that do not contain the AQUA peptide sequence.

The AQUA methodology employs the introduction of a known quantity of at least one heavy-isotope labeled peptide standard (which has a unique signature detectable by LC-SRM chromatography) into a digested biological sample in order to determine, by comparison to the peptide standard, the absolute quantity of a peptide with the same sequence and protein modification in the biological sample. Briefly, the AQUA methodology has two stages: peptide internal standard selection and validation and method development; and implementation using validated peptide internal standards to detect and quantify a target protein in sample. The method is a powerful technique for detecting and quantifying a given peptide/protein within a complex biological mixture, such as a cell lysate, and may be employed, e.g., to quantify change in protein phosphorylation as a result of drug treatment, or to quantify differences in the level of a protein in different biological states.

Generally, to develop a suitable internal standard, a particular peptide (or modified peptide) within a target protein sequence is chosen based on its amino acid sequence and the particular protease to be used to digest. The peptide is then generated by solid-phase peptide synthesis such that one residue is replaced with that same residue containing stable isotopes (¹³C, ¹⁵N). The result is a peptide that is chemically identical to its native counterpart formed by proteolysis, but is easily distinguishable by MS via a 7-Da mass shift. The newly synthesized AQUA internal standard peptide is then evaluated by LC-MS/MS. This process provides qualitative information about peptide retention by reverse-phase chromatography, ionization efficiency, and fragmentation via collision-induced dissociation. Informative and abundant fragment ions for sets of native and internal standard peptides are chosen and then specifically monitored in rapid succession as a function of chromatographic retention to form a selected reaction monitoring (LC-SRM) method based on the unique profile of the peptide standard.

The second stage of the AQUA strategy is its implementation to measure the amount of a protein or modified protein from complex mixtures. Whole cell lysates are typically fractionated by SDS-PAGE gel electrophoresis, and regions of the gel consistent with protein migration are excised. This process is followed by in-gel proteolysis in the presence of the AQUA peptides and LC-SRM analysis. (See Gerber et al., supra.) AQUA peptides are spiked in to the complex peptide mixture obtained by digestion of the whole cell lysate with a proteolytic enzyme and subjected to immunoaffinity purification as described above. The retention time and fragmentation pattern of the native peptide formed by digestion (e.g., trypsinization) is identical to that of the AQUA internal standard peptide determined previously; thus, LC-MS/MS analysis using an SRM experiment results in the highly specific and sensitive measurement of both internal standard and analyte directly from extremely complex peptide mixtures.

Since an absolute amount of the AQUA peptide is added (e.g., 250 fmol), the ratio of the areas under the curve can be used to determine the precise expression levels of a protein or phosphorylated form of a protein in the original cell lysate. In addition, the internal standard is present during in-gel digestion as native peptides are formed, such that peptide extraction efficiency from gel pieces, absolute losses during sample handling (including vacuum centrifugation), and variability during introduction into the LC-MS system do not affect the determined ratio of native and AQUA peptide abundances.

An AQUA peptide standard is developed for a known sequence previously identified by the IAP-LC-MS/MS method within in a target protein. If the site is modified, one AQUA peptide incorporating the modified form of the particular residue within the site may be developed, and a second AQUA peptide incorporating the unmodified form of the residue developed. In this way, the two standards may be used to detect and quantify both the modified an unmodified forms of the site in a biological sample.

Peptide internal standards may also be generated by examining the primary amino acid sequence of a protein and determining the boundaries of peptides produced by protease cleavage. Alternatively, a protein may actually be digested with a protease and a particular peptide fragment produced can then sequenced. Suitable proteases include, but are not limited to, serine proteases (e.g. trypsin, hepsin), metallo proteases (e.g., PUMP1), chymotrypsin, cathepsin, pepsin, thermolysin, carboxypeptidases, etc.

A peptide sequence within a target protein is selected according to one or more criteria to optimize the use of the peptide as an internal standard. Preferably, the size of the peptide is selected to minimize the chances that the peptide sequence will be repeated elsewhere in other non-target proteins. Thus, a peptide is preferably at least about 6 amino acids. The size of the peptide is also optimized to maximize ionization frequency. Thus, in some embodiments, the peptide is not longer than about 20 amino acids. In some embodiments, the peptide is between about 7 to 15 amino acids in length. A peptide sequence is also selected that is not likely to be chemically reactive during mass spectrometry, thus sequences comprising cysteine, tryptophan, or methionine are avoided.

A peptide sequence that does not include a modified region of the target region may be selected so that the peptide internal standard can be used to determine the quantity of all forms of the protein. Alternatively, a peptide internal standard encompassing a modified amino acid may be desirable to detect and quantify only the modified form of the target protein. Peptide standards for both modified and unmodified regions can be used together, to determine the extent of a modification in a particular sample (i.e. to determine what fraction of the total amount of protein is represented by the modified form). For example, peptide standards for both the phosphorylated and unphosphorylated form of a protein known to be phosphorylated at a particular site can be used to quantify the amount of phosphorylated form in a sample.

The peptide is labeled using one or more labeled amino acids (i.e., the label is an actual part of the peptide) or less preferably, labels may be attached after synthesis according to standard methods. Preferably, the label is a mass-altering label selected based on the following considerations: The mass should be unique to shift fragments masses produced by MS analysis to regions of the spectrum with low background; the ion mass signature component is the portion of the labeling moiety that preferably exhibits a unique ion mass signature in MS analysis; the sum of the masses of the constituent atoms of the label is preferably uniquely different than the fragments of all the possible amino acids. As a result, the labeled amino acids and peptides are readily distinguished from unlabeled ones by the ion/mass pattern in the resulting mass spectrum. Preferably, the ion mass signature component imparts a mass to a protein fragment that does not match the residue mass for any of the 20 natural amino acids.

The label should be robust under the fragmentation conditions of MS and not undergo unfavorable fragmentation. Labeling chemistry should be efficient under a range of conditions, particularly denaturing conditions, and the labeled tag preferably remains soluble in the MS buffer system of choice. The label preferably does not suppress the ionization efficiency of the protein and is not chemically reactive. The label may contain a mixture of two or more isotopically distinct species to generate a unique mass spectrometric pattern at each labeled fragment position. Stable isotopes, such as ²H, ¹³C, ¹⁵N, ¹⁷O, ¹⁸O, or ³⁴S, are some non-limiting labels. Pairs of peptide internal standards that incorporate a different isotope label may also be prepared. Non-limiting amino acid residues into which a heavy isotope label may be incorporated include leucine, proline, valine, and phenylalanine.

Peptide internal standards are characterized according to their mass-to-charge (m/z) ratio, and preferably, also according to their retention time on a chromatographic column (e.g., an HPLC column). Internal standards that co-elute with unlabeled peptides of identical sequence are selected as optimal internal standards. The internal standard is then analyzed by fragmenting the peptide by any suitable means, for example by collision-induced dissociation (CID) using, e.g., argon or helium as a collision gas. The fragments are then analyzed, for example by multi-stage mass spectrometry (MSⁿ) to obtain a fragment ion spectrum, to obtain a peptide fragmentation signature. Preferably, peptide fragments have significant differences in m/z ratios to enable peaks corresponding to each fragment to be well separated, and a signature is that is unique for the target peptide is obtained. If a suitable fragment signature is not obtained at the first stage, additional stages of MS are performed until a unique signature is obtained.

Fragment ions in the MS/MS and MS³ spectra are typically highly specific for the peptide of interest, and, in conjunction with LC methods, allow a highly selective means of detecting and quantifying a target peptide/protein in a complex protein mixture, such as a cell lysate, containing many thousands or tens of thousands of proteins. Any biological sample potentially containing a target protein/peptide of interest may be assayed. Crude or partially purified cell extracts are preferably employed. Generally, the sample has at least 0.01 mg of protein, typically a concentration of 0.1-10 mg/mL, and may be adjusted to a desired buffer concentration and pH.

A known amount of a labeled peptide internal standard, preferably about 10 femtomoles, corresponding to a target protein to be detected/quantified is then added to a biological sample, such as a cell lysate. The spiked sample is then digested with one or more protease(s) for a suitable time period to allow digestion. A separation is then performed (e.g. by HPLC, reverse-phase HPLC, capillary electrophoresis, ion exchange chromatography, etc.) to isolate the labeled internal standard and its corresponding target peptide from other peptides in the sample. Microcapillary LC is a one non-limiting method.

Each isolated peptide is then examined by monitoring of a selected reaction in the MS. This involves using the prior knowledge gained by the characterization of the peptide internal standard and then requiring the MS to continuously monitor a specific ion in the MS/MS or MSⁿ spectrum for both the peptide of interest and the internal standard. After elution, the area under the curve (AUC) for both peptide standard and target peptide peaks are calculated. The ratio of the two areas provides the absolute quantification that can be normalized for the number of cells used in the analysis and the protein's molecular weight, to provide the precise number of copies of the protein per cell. Further details of the AQUA methodology are described in Gygi et al., and Gerber et al. supra.

AQUA internal peptide standards (heavy-isotope labeled peptides) may desirably be produced, as described above, to detect any quantify any unique site (e.g., the fusion junction within a ROS or ALK fusion polypeptide) within a polypeptide of the invention. For example, an AQUA phosphopeptide may be prepared that corresponds to the fusion junction sequence of one of the ROS or ALK fusion polypeptides. Peptide standards for may be produced for the fusion junction and such standards employed in the AQUA methodology to detect and quantify the fusion junction (i.e. the presence of that fusion polypeptide) in a biological sample.

For example, one non-limiting AQUA peptide of the invention comprises the amino acid sequence AGSTLP (SEQ ID NO: 29), which corresponds to the three amino acids immediately flanking each side of the fusion junction in the short variant of FIG-ROS fusion polypeptide (i.e., FIG-ROS(S) fusion ppolypeptide), where the amino acids encoded by the FIG gene are italicized and the amino acids encoded by the ROS gene in bold. It will be appreciated that larger AQUA peptides comprising the fusion junction sequence (and additional residues downstream or upstream of it) may also be constructed. Similarly, a smaller AQUA peptide comprising less than all of the residues of such sequence (but still comprising the point of fusion junction itself) may alternatively be constructed. Such larger or shorter AQUA peptides are within the scope of the present invention, and the selection and production of AQUA peptides may be carried out as described above (see Gygi et al., Gerber et al., supra.).

It should be noted that because the sequence of the AQUA peptide spanning the fusion junction of one of the ROS fusion proteins described herein may also be (or be included in) the epitope to which a ROS fusion-specific antibody specifically binds. An “epitope” refers to either an immunogenic epitope (i.e., capable of eliciting an immune response) or an antigenic epitope (i.e., the region of a protein molecule to which an antibody can specifically bind. The number of immunogenic epitopes of a protein generally is less than the number of antigenic epitopes. See, for instance, Geysen et al., Proc. Natl. Acad. Sci. USA 81:3998-4002 (1983).

Table 3 provides a list of the sequences of all the fusion junctions of the ROS fusion polypeptides of the invention, where the amino acids encoded by the non-ROS gene are italicized and the amino acids encoded by the ROS gene in bold.

TABLE 3

| | Junction | |
|---|---|---|
| Fusion | Sequence | SEQ ID NO: |
| SLC34A2-ROS | VGVWHR | 25 |
| (very short) | | |
| SLC34A2-ROS | LVGDDF | 26 |
| (short) | | |
| SLC34A2-ROS | LVGAGV | 27 |
| (long) | | |
| CD74-ROS | PPKDDF | 28 |
| FIG-ROS | AGSTLP | 29 |
| (short) | | |
| FIG-ROS | LQVWHR | 30 |
| (long) | | |
| FIG-ROS | VLQAGV | 31 |
| (Extra Long) | | |

In some embodiments, the mammalian cancer is from a human. In various embodiments, the biological sample is from the cancer or suspected cancer of the patient. In some embodiments, the cancer is a solid tumor cancer. In some embodiments, the cancer is leukemia. In some embodiments, the cancer is lymphoma. In some embodiments, the cancer is a lung cancer (e.g., a non-small cell lung carcinoma or a small cell lung carcinoma). In some embodiments, the cancer is a brain cancer (e.g., glioblastoma). In some embodiments, the cancer is a liver cancer (e.g., cholangiocarcinoma). In some embodiments, the cancer is colon cancer. In some embodiments, the cancer is breast cancer. In some embodiments, the cancer is ovarian cancer.

In some embodiments, the mammalian lung cancer is NSCLC (non-small cell lung carcinoma). In some embodiments, the mammalian lung cancer is SCLC (small cell lung carcinoma). In further embodiments of the methods of the invention, the mammal is a human, and the human may be a candidate for a ROS-inhibiting therapeutic, an ALK-inhibiting therapeutic, or both, for the treatment of a lung cancer. The human candidate may be a patient currently being treated with, or considered for treatment with, an ROS kinase inhibitor. In another embodiment, the mammal is large animal, such as a horse or cow, while in other embodiments, the mammal is a small animal, such as a dog or cat, all of which are known to develop lung cancers, such as NSCLC and SCLC.

As used throughout the specification, the term “biological sample” is used in its broadest sense, and means any biological sample suspected of containing a polypeptide with ROS kinase activity or a polypeptide with ALK kinase activity including, without limitation, a ROS or ALK fusion polypeptide or a full length ROS or ALK protein (with or without the signal peptide sequence) or fragments having ROS or ALK kinase activity thereof. Biological samples include, without limitation, saliva, mucous, tears, blood, circulating tumor cells, serum, tissues, bone marrow, lymph/interstitial fluids, buccal cells, mucosal cells, cerebrospinal fluid, semen, feces, plasma, urine, a suspension of cells, or a suspension of cells and viruses or extracts thereof, and may comprise a cell, chromosomes isolated from a cell (e.g., a spread of metaphase chromosomes), genomic DNA (in solution or bound to a solid support such as for Southern analysis), RNA (in solution or bound to a solid support such as for northern analysis), cDNA (in solution or bound to a solid support). In some embodiments, the biological sample contains lung cells suspected of being cancerous.

Any biological sample comprising cells (or extracts of cells) from a mammalian cancer is suitable for use in the methods of the invention. In one embodiment, the biological sample comprises cells obtained from a tumor biopsy or a tumor resection. The biopsy or resection may be obtained, according to standard clinical techniques, from primary tumors occurring in an organ of a mammal, or by secondary tumors that have metastasized in other tissues. In another embodiment, the biological sample comprises cells obtained from a fine needle aspirate taken from a tumor, and techniques for obtaining such aspirates are well known in the art (see Cristallini et al., Acta Cytol. 36(3): 416-22 (1992)).

The biological sample may also comprise cells obtained from an effusion, such as a pleural effusion. Pleural effusions (liquid that forms outside the lung in the thoracic cavity and which contains cancerous cells) are known to form in many patients with advanced lung cancer (including NSCLC), and the presence of such effusion is predictive of a poor outcome and short survival time. Standard techniques for obtaining pleural effusion samples have been described and are well known in the art (see Sahn, Clin Chest Med. 3(2): 443-52 (1982)).

The biological sample may comprise cells obtained from a bronchoalveolar lavage. Bronchoalveolar lavage is a standard medical procedure in which a bronchoscope is passed through the mouth or nose into the lungs and fluid is squirted into a small part of the lung and then recollected for examination.

In some embodiments, the biological sample comprises circulating tumor cells. Circulating tumor cells (“CTCs”) may be purified, for example, using the kits and reagents sold under the trademarks Vita-Assays™, Vita-Cap™, and CellSearch® (commercially available from Vitatex, LLC (a Johnson and Johnson corporation). Other methods for isolating CTCs are described (see, for example, PCT Publication No. WO/2002/020825, Cristofanilli et al., New Engl. J. of Med. 351 (8):781-791 (2004), and Adams et al., J. Amer. Chem. Soc. 130(27): 8633-8641 (July 2008)). In a particular embodiment, a circulating tumor cell (“CTC”) may be isolated and identified as having originated from the lung.

Accordingly, the invention provides a method for isolating a CTC, and then screening the CTC one or more assay formats to identify the presence of a polypeptide with ROS kinase activity or nucleic acid molecule encoding the same in the CTC.

Cellular extracts of the biological samples described herein may be prepared, either crude or partially (or entirely) purified, in accordance with standard techniques, and used in the methods of the invention. Alternatively, biological samples comprising whole cells may be utilized in assay formats such as in vitro kinase assay, ELISA assays, immunohistochemistry (IHC), flow cytometry (FC), and immunofluorescence (IF), immuno-histochemistry (HC), fluorescence in situ hybridization (FISH) and polymerase chain reaction (PCR), according to standard methods such as those described below (see, also, e.g., Ausubel et al., supra). Such whole-cell assays are advantageous in that they minimize manipulation of the tumor cell sample and thus reduce the risks of altering the in vivo signaling/activation state of the cells and/or introducing artifact signals. Whole cell assays are also advantageous because they characterize expression and signaling only in tumor cells, rather than a mixture of tumor and normal cells.

Thus, biological samples useful in the practice of the methods of the invention may be obtained from any mammal in which a cancer or suspected cancer characterized by the presence of a polypeptide having ROS kinase activity or a polypeptide having ALK kinase activity is present or might be present or developing. As used herein, the phrase “characterized by” with respect to a cancer (or suspected cancer) and indicated molecule (e.g., a polypeptide with ROS kinase activity or a polypeptide with ALK kinase activity) is meant a cancer (or suspected cancer) in which a gene translocation or mutation (e.g., causing aberrant expression of full-length ROS or ALK) and/or an expressed polypeptide with ROS kinase activity or ALK kinase activity is present, as compared to another cancer or a normal tissue in which such translocation or aberrant expression is not present. The presence of such translocation, aberrant expression of a polypeptide with ROS kinase activity or a polypeptide with ALK kinase activity may drive (i.e., stimulate or be the causative agent of), in whole or in part, the growth and survival of such cancer or suspected cancer. However, since ALK kinase activity is never observed in the same cell as ROS kinase activity, only one of either ALK or ROS will drive a particular cancer.

Accordingly, any biological sample (e.g., CTC, pleural effusion, needle aspirate, tumor biopsy, etc. . . . ) from a patient that is identified as comprising a polypeptide with ROS kinase activity or polynucleotide encoding the same (e.g., a full length ROS polypeptide or polynucleotide or a ROS fusion polypeptide or polynucleotide) may indicate that the patient's originating cancer (e.g., an lung cancer such as NSCLC or SCLC) is being driven by the polypeptide with ROS kinase activity and thus is likely to respond to a composition comprising at least one ROS kinase-inhibiting therapeutic.

As used herein, by “likely to respond” is meant that a cancer is more likely to show growth retardation or abrogation in response to (e.g., upon contact with or treatment by) a ROS inhibiting therapeutic. In some embodiments, a cancer that is likely to respond to a ROS inhibiting therapeutic is one that dies (e.g., the cancer cells apoptose) in response to the ROS inhibiting therapeutic.

In assessing the presence of a polypeptide with ROS kinase activity or a polypeptide with ALK kinase activity (or polynucleotides encoding the same) in a biological sample comprising cells from a mammalian cancer tumor, a control sample representing a cell in which such a polypeptide does not occur (e.g., healthy lung cells) may desirably be employed for comparative purposes. Ideally, the control sample comprises cells from a subset of the particular cancer (e.g., lung cancer) that is representative of the subset in which the polypeptide (or polynucleotide encoding the same) does not occur. Comparing the level in the control sample versus the test biological sample thus identifies whether the mutant polynucleotide and/or polypeptide is/are present. Alternatively, since a polypeptide with ROS kinase activity or a polypeptide with ALK kinase activity (or polynucleotides encoding the same) may not be present in the majority of cancers, any tissue that similarly does not express polypeptide with ROS kinase activity or a polypeptide with ALK kinase activity (or polynucleotides encoding the same) may be employed as a control.

The methods described herein will have valuable diagnostic utility for cancers characterized by the presence of a polypeptide with ROS kinase activity or a polypeptide with ALK kinase activity, and treatment decisions pertaining to the same. For example, biological samples may be obtained from a subject that has not been previously diagnosed as having a cancer characterized by the presence of polypeptide with ROS kinase activity, nor has yet undergone treatment for such cancer, and the method is employed to diagnostically identify a tumor in such subject as belonging to a subset of tumors (e.g., NSCLC or SCLC) in which a polypeptide with ROS kinase activity (or polynucleotide encoding the same) is present/expressed.

Alternatively, a biological sample may be obtained from a subject that has been diagnosed as having a cancer characterized by the presence of one type of kinase, such as EFGR, and has been receiving therapy, such as EGFR inhibitor therapy (e.g., Tarceva™, Iressa™) for treatment of such cancer, and the method of the invention is employed to identify whether the subject's tumor is also characterized by the presence of polypeptide with ROS kinase activity (or polynucleotide encoding the same) such as full length ROS protein or one of the many ROS fusion polypeptides (e.g., SLC34A2-ROS(S)), and is therefore likely to fully respond to the existing therapy and/or whether alternative or additional ROS-inhibiting therapy is desirable or warranted. The methods of the invention may also be employed to monitor the progression or inhibition of a polypeptide with ROS kinase activity-expressing cancer following treatment of a subject with a composition comprising a ROS-inhibiting therapeutic or combination of therapeutics.

Such diagnostic assay may be carried out subsequent to or prior to preliminary evaluation or surgical surveillance procedures. The identification method of the invention may be advantageously employed as a diagnostic to identify patients having cancer, such as lung cancer (e.g., non-small cell lung cancer) or colon cancer, characterized by the presence of a polypeptide with ROS kinase activity or A LK kinase activity, which patients would be most likely to respond to therapeutics targeted at inhibiting ROS or ALK kinase activity. The ability to select such patients would also be useful in the clinical evaluation of efficacy of future ROS- or ALK-inhibiting therapeutics as well as in the future prescription of such drugs to patients.

The ability to selectively identify cancers in which a polypeptide with ROS kinase activity (or polynucleotide encoding the same) or a polypeptide with ALK kinase activity (or polypeptide encoding the same) is/are present enables important new methods for accurately identifying such tumors for diagnostic purposes, as well as obtaining information useful in determining whether such a tumor is likely to respond to a ROS- and/or ALK-inhibiting therapeutic composition, or likely to be partially or wholly non-responsive to an inhibitor targeting a different kinase when administered as a single agent for the treatment of the cancer.

As used herein, by “cancer” or “cancerous” is meant a cell that shows abnormal growth as compared to a normal (i.e., non-cancerous) cell of the same cell type. For example, a cancerous cell may be metastatic or non-metastatic. A cancerous cell may also show lack of contact inhibition where a normal cell of that same cell type shows contact inhibition. In some embodiments, the cancer is lung cancer (e.g., non-small cell lung cancer or small cell lung cancer). As used herein, by “suspected cancer” (as in “suspected mammalian lung cancer”) or “tissue suspected of being cancerous” is meant a cell or tissue that has some aberrant characteristics (e.g., hyperplastic or lack of contact inhibition) as compared to normal cells or tissues of that same cell or tissue type as the suspected cancer, but where the cell or tissue is not yet confirmed by a physician or pathologist as being cancerous.

In some embodiments, the various methods of the invention may be carried out in a variety of different assay formats known to those of skill in the art. Some non-limiting examples of methods include immunoassays and peptide and nucleotide assays.

### Immunoassays.

Immunoassays useful in the practice of the methods of the invention may be homogenous immunoassays or heterogeneous immunoassays. In a homogeneous assay the immunological reaction usually involves a specific reagent (e.g. a ROS-specific antibody or an ALK-specific antibody), a labeled analyte, and the biological sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radio-isotopes, fluorescent dyes, enzymes, bacteriophages, coenzymes, and so forth. Semi-conductor nanocrystal labels, or “quantum dots”, may also be advantageously employed, and their preparation and use has been well described. See generally, K. Barovsky, Nanotech. Law & Bus. 1(2): Article 14 (2004) and patents cited therein.

In a heterogeneous assay approach, the materials are usually the biological sample, binding reagent (e.g., an antibody), and suitable means for producing a detectable signal. Biological samples as further described below may be used. The antibody is generally immobilized on a support, such as a bead, plate or slide, and contacted with the sample suspected of containing the antigen in a liquid phase. The support is then separated from the liquid phase and either the support phase or the liquid phase is examined for a detectable signal employing means for producing such signal. The signal is related to the presence of the analyte in the biological sample. Means for producing a detectable signal include the use of radioactive labels, fluorescent labels, enzyme labels, quantum dots, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof, which may be useful for carrying out the methods disclosed herein, are well known in the art. See generally E. Maggio, Enzyme-Immunoassay, (1980) (CRC Press, Inc., Boca Raton, Fla.); see also, e.g., U.S. Pat. No. 4,727,022 (Skold et al., “Methods for Modulating Ligand-Receptor Interactions and their Application”); U.S. Pat. No. 4,659,678 (Forrest et al., “Immunoassay of Antigens”); U.S. Pat. No. 4,376,110 (David et al., “Immunometric Assays Using Monoclonal Antibodies”). Conditions suitable for the formation of reagent-antibody complexes are well known to those of skill in the art. See id. ROS-specific antibodies may be used in a “two-site” or “sandwich” assay, with a single hybridoma cell line serving as a source for both the labeled monoclonal antibody and the bound monoclonal antibody. Such assays are described in U.S. Pat. No. 4,376,110. The concentration of detectable reagent should be sufficient such that the binding of a protein with ROS kinase activity (e.g., a full-length ROS protein or a ROS fusion polypeptide) is detectable compared to background.

Antibodies useful in the practice of the methods disclosed herein may be conjugated to a solid support suitable for a diagnostic assay (e.g., beads, plates, slides or wells formed from materials such as latex or polystyrene) in accordance with known techniques, such as precipitation. Antibodies or other binding reagents binding reagents may likewise be conjugated to detectable groups such as radiolabels (e.g., ³⁵S, ¹²⁵I, ¹³¹I), enzyme labels (e.g., horseradish peroxidase, rosaline phosphatase), and fluorescent labels (e.g., fluorescein) in accordance with known techniques.

Cell-based assays, such flow cytometry (FC), immuno-histochemistry (IHC), or immunofluorescence (IF) are particularly desirable in practicing the methods of the invention, since such assay formats are clinically-suitable, allow the detection of expression of a protein with ROS kinase activity or a protein with ALK kinase activity in vivo, and avoid the risk of artifact changes in activity resulting from manipulating cells obtained from, e.g. a tumor sample in order to obtain extracts. Accordingly, in some embodiments, the methods of the invention are implemented in a flow-cytometry (FC), immuno-histochemistry (IHC), or immunofluorescence (IF) assay format.

Flow cytometry (FC) may be employed to determine the expression of polypeptide with ROS kinase activity or ALK kinase activity in a mammalian tumor before, during, and after treatment with a drug targeted at inhibiting ROS and/or ALK kinase activity. For example, tumor cells from a fine needle aspirate may be analyzed by flow cytometry for expression and/or activation of a polypeptide with ROS kinase activity or ALK kinase activity or polynucleotide encoding the same, as well as for markers identifying cancer cell types, etc., if so desired. Flow cytometry may be carried out according to standard methods. See, e.g. Chow et al., Cytometry (Communications in Clinical Cytometry) 46: 72-78 (2001). Briefly and by way of example, the following protocol for cytometric analysis may be employed: fixation of the cells with 2% paraformaldehyde for 10 minutes at 37° C. followed by permeabilization in 90% methanol for 10 minutes on ice. Cells may then be stained with the primary antibody (e.g., a full-length ROS-specific or a ROS fusion polypeptide-specific antibody), washed and labeled with a fluorescent-labeled secondary antibody. The cells would then be analyzed on a flow cytometer (e.g. a Beckman Coulter FC500) according to the specific protocols of the instrument used. Such an analysis would identify the level of expressed full-length ROS or ALK or a ROS fusion or ALK fusion polypeptide in the tumor. Similar analysis after treatment of the tumor with a ROS- and/or ALK-inhibiting therapeutic would reveal the responsiveness of the tumor to the targeted inhibitor of ROS or ALK kinase.

Immunohistochemical (IHC) staining may be also employed to determine the expression and/or activation status of polypeptide with ROS kinase activity in a mammalian cancer (e.g., a lung cancer) before, during, and after treatment with a therapeutic targeted at inhibiting ROS kinase activity. IHC may be carried out according to well-known techniques. See, e.g., ANTIBODIES: A LABORATORY MANUAL, Chapter 10, Harlow & Lane Eds., Cold Spring Harbor Laboratory (1988). Briefly, and by way of example, paraffin-embedded tissue (e.g. tumor tissue from a biopsy) is prepared for immunohistochemical staining by deparaffinizing tissue sections with xylene followed by ethanol; hydrating in water then PBS; unmasking antigen by heating slide in sodium citrate buffer, incubating sections in hydrogen peroxide; blocking in blocking solution; incubating slide in primary antibody (e.g., a ROS-specific antibody) and secondary antibody; and finally detecting using avidin/biotin method.

Immunofluorescence (IF) assays may be also employed to determine the expression and/or activation status of a polypeptide with ROS kinase activity (e.g., full length ROS polypeptide or a ROS fusion polypeptide) in a mammalian cancer before, during, and after treatment with a therapeutic targeted at inhibiting ROS kinase activity. IF may be carried out according to well-known techniques. See, e.g., J. M. polak and S. Van Noorden (1997) INTRODUCTION TO IMMUNOCYTOCHEMISTRY, 2nd Ed.; ROYAL MICROSCOPY SOCIETY MICROSCOPY HANDBOOK 37, BioScientific/Springer-Verlag. Briefly, and by way of example, patient samples may be fixed in paraformaldehyde followed by methanol, blocked with a blocking solution such as horse serum, incubated with a primary antibody against (i.e., that specifically binds to) a polypeptide with ROS kinase activity (e.g., a CD74-ROS fusion polypeptide) or a polypeptide with ALK kinase activity (e.g., an EML4-ALK fusion polypeptide) followed by a secondary antibody labeled with a fluorescent dye such as Alexa 488 and analyzed with an epifluorescent microscope.

A variety of other protocols, including enzyme-linked immunosorbent assay (ELISA), radio-immunoassay (RIA), Western blotting analysis, in vitro kinase assay, and fluorescent-activated cell sorting (FACS), for measuring expression and/or activity of a polypeptide with ROS kinase activity are known in the art and provide a basis for diagnosing the presence of the polypeptide with ROS kinase activity (e.g., a full-length ROS, or an ROS fusion polypeptide such as an FIG-ROS(S) fusion polypeptide) or the presence of a polypeptide with ALK kinase activity (e.g., full length ALK or an ALK fusion polypeptide such as NPM-ALK fusion polypeptide). Normal or standard values for ALK or ROS (full length or fusion) polypeptide expression are established by combining body fluids or cell extracts taken from normal mammalian subjects, preferably human, with an antibody that specifically binds to a polypeptide with ROS kinase activity or a polypeptide with ALK kinase activity under conditions suitable for complex formation. The amount of standard complex formation may be quantified by various methods, but preferably by photometric means. Quantities of full length ROS polypeptide expressed in subject, control, and disease samples from biopsied tissues are compared with the standard values. Deviation between standard and subject values establishes the parameters for diagnosing disease. Note that in some tissues (e.g., lung cancer) since the proteins with ROS kinase activity or proteins with ALK kinase activity (e.g., SLC34A2-ROS(S) and EML4-ALK (796aa variant)) were discovered in cancerous tissue, no normal lung tissue biological samples are expected to contain these proteins with ROS kinase activity or ALK kinase activity (or polynucleotides encoding the same).

In another aspect, the invention provides a method for detecting the presence of a polynucleotide encoding a polypeptide with ROS kinase activity or ALK kinase activity in a biological sample from a mammalian lung cancer or suspected mammalian lung cancer, said method comprising the steps of: (a) obtaining a biological sample from a mammalian lung cancer or suspected mammalian lung cancer and (b) utilizing a reagent that specifically binds to said polynucleotide encoding said polypeptide to determine whether said polynucleotide is present in said biological sample, wherein detection of specific binding of said reagent to said biological sample indicates said polynucleotide encoding said polypeptide with ROS kinase activity or ALK kinase activity is present in said biological sample.

The presence of a polynucleotide encoding a polypeptide having ROS kinase activity or ALK kinase activity can be assessed by any standard methods. In addition, these methods can be combined with methods to detect the polypeptide having ROS kinase activity or ALK kinase activity as described above.

### Nucleotide Assays.

Full length ROS polynucleotide or ROS fusion polynucleotide-specific binding reagents and full length ALK polynucleotide or ALK fusion polynucleotide-specific binding reagents useful in practicing the methods of the invention may also be mRNA, oligonucleotide or DNA probes that can directly hybridize to, and detect, fusion or truncated polypeptide expression transcripts in a biological sample. Such probes are discussed in detail herein. Briefly, and by way of example, formalin-fixed, paraffin-embedded (PPFE) patient samples may be probed with a fluorescein-labeled RNA probe followed by washes with formamide, SSC and PBS and analysis with a fluorescent microscope.

Polynucleotides encoding a polypeptide with ROS or ALK kinase activity may also be used for diagnostic purposes. The polynucleotides that may be used include oligonucleotide sequences, antisense RNA and DNA molecules, and PNAs. The polynucleotides may be used to detect and quantitate gene expression in biopsied tissues in which expression of a polypeptide with ROS or ALK kinase activity (e.g., a ROS or ALK fusion polypeptide or full length ROS or ALK) may be correlated with disease. The diagnostic assay may be used to distinguish between absence, presence, and excess expression of a polypeptide with ROS or ALK kinase activity, and to monitor regulation of levels of a polypeptide with ROS or ALK kinase activity during therapeutic intervention.

In one embodiment, hybridization with PCR primers which are capable of detecting polynucleotide sequences, including genomic sequences, encoding a polypeptide with ROS or ALK kinase activity may be used to identify nucleic acid sequences that encode such polypeptides with ROS or ALK kinase activity. The specificity of the probe, whether it is made from a highly specific region, e.g., 10 unique nucleotides in the fusion junction, or a less specific region, e.g., the 3′ coding region, and the stringency of the hybridization or amplification (maximal, high, intermediate, or low) will determine whether the probe identifies only naturally occurring sequences encoding ROS or ALK kinase polypeptides (e.g., full length ROS or ALK or a ROS or ALK fusion protein), alleles, or related sequences.

Probes may also be used for the detection of related sequences, and should preferably contain at least 50% of the nucleotides from any of the ROS polypeptide- or ALK polypeptide-encoding sequences. The hybridization probes (e.g., FISH probes or Southern or Northern blotting probes) of the subject invention may be DNA or RNA and derived from the nucleotide sequences of encoding polypeptides with ROS kinase activity and polypeptides with ALK kinase activity. In some embodiments, where the polypeptide having ROS or ALK kinase activity is a fusion protein, the hybridization probes encompassing the fusion junction, or from genomic sequence including promoter, enhancer elements, and introns of the naturally occurring ROS or ALK gene and the fusion partner gene (e.g., for ROS, SLC34A2, FIG, or CD74; for ALK, NPM, EML4, TFG, etc.).

A ROS fusion polynucleotide (i.e., a polynucleotide encoding a ROS fusion polypeptide such as FIG-ROS(S) or CD74-ROS), full length ROS polynucleotide, ALK fusion polynucleotide (i.e., a polynucleotide encoding an ALK fusion polynucleotide such as EML4-ALK (796 as variant) or full length ALK polynucleotide may be used in Southern or Northern analysis, dot blot, or other membrane-based technologies; in PCR technologies; or in dip stick, pin, ELISA or chip assays utilizing fluids or tissues from patient biopsies to detect altered expression of a polypeptide with ROS kinase activity. Such qualitative or quantitative methods are well known in the art. In a particular aspect, the nucleotide sequences encoding a polypeptide with ROS or ALK kinase activity may be useful in assays that detect activation or induction of various cancers, including lung cancer (e.g., non-small cell lung carcinoma (NSCLC) and small cell lung carcinoma) and colon cancer. Polynucleotides encoding a polypeptide with ROS kinase activity may be detectably labeled by standard methods, and added to a fluid or tissue sample from a patient under conditions suitable for the formation of hybridization complexes. After a suitable incubation period, the sample is washed and the signal is quantitated and compared with a standard value. If the amount of signal in the biopsied or extracted sample is significantly altered from that of a comparable control sample, the nucleotide sequences have hybridized with nucleotide sequences in the sample, and the presence of altered levels of nucleotide sequences encoding a polypeptide with ROS or ALK kinase activity (e.g., a ROS or ALK fusion polypeptide or full length ROS or ALK polypeptide) in the sample indicates the presence of the associated disease. Such assays may also be used to evaluate the efficacy of a particular therapeutic treatment regimen in animal studies, in clinical trials, or in monitoring the treatment of an individual patient.

In some embodiments, the methods of the invention are carried out using a PCR assay format. Polymerase chain reaction (PCR) is standard to those of skill in the art. See, e.g., MOLECULAR CLONING, A LABORATORY MANUAL, 2nd. edition, Sambrook, J., Fritsch, E. F. and Maniatis, T., eds., Cold Spring Harbor Laboratory Press, Cold Spring Harbor, N.Y. (1989). PCR primers (also called oligomers) may be chemically synthesized, generated enzymatically, or produced from a recombinant source. Oligomers will preferably consist of two nucleotide sequences, one with sense orientation (5′ to 3) and another with antisense (3′ to 5′), employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

Methods which may also be used to quantitate the expression of a polypeptide with ROS or ALK kinase activity (e.g., ROS or ALK fusion polypeptide or full ROS or ALK polypeptide) include radiolabeling or biotinylating nucleotides, coamplification of a control nucleic acid, and standard curves onto which the experimental results are interpolated (Melby et al., J. Immunol. Methods, 159: 235-244 (1993); Duplaa et al. Anal. Biochem. 229-236 (1993)). The speed of quantitation of multiple samples may be accelerated by running the assay in an ELISA format where the oligomer of interest is presented in various dilutions and a spectrophotometric or colorimetric response gives rapid quantitation.

In another embodiment of the invention, the polynucelotides encoding a polypeptide with ROS or ALK kinase activity may be used to generate hybridization probes which are useful for mapping the naturally occurring genomic sequence. The sequences may be mapped to a particular chromosome or to a specific region of the chromosome using well known techniques. Such techniques include fluorescence in-situ hybridization (FISH), FACS, or artificial chromosome constructions, such as yeast artificial chromosomes, bacterial artificial chromosomes, bacterial P1 constructions or single chromosome cDNA libraries, as reviewed in Price, C. M., Blood Rev. 7: 127-134 (1993), and Trask, B. J., Trends Genet. 7: 149-154 (1991).

In further embodiments, fluorescence in-situ hybridization (FISH) is employed in the methods of the invention (as described in Verma et al. HUMAN CHROMOSOMES: A MANUAL OF BASIC TECHNIQUES, Pergamon Press, New York, N.Y. (1988)). In some embodiments, the FISH assay may be correlated with other physical chromosome mapping techniques and genetic map data. The FISH technique is well known (see, e.g., U.S. Pat. Nos. 5,756,696; 5,447,841; 5,776,688; and 5,663,319). Examples of genetic map data can be found in the 1994 Genome Issue of Science (265: 1981f). Correlation between the location of the gene encoding ROS or ALK protein and/or, in the case of fusion polypeptides, the gene encoding the fusion partner of a ROS or ALK fusion protein (e.g., for ROS, the FIG gene, the SLC34A2 gene, or the CD74 gene; for ALK, the EML4 gene, the NPM gene, the ATIC gene, the CARS gene, etc. . . . ) on a physical chromosomal map and a specific disease, or predisposition to a specific disease, may help delimit the region of DNA associated with that genetic disease. The nucleotide sequences of the subject invention may be used to detect differences in gene sequences between normal, carrier, or affected individuals.

In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (Gatti et al., Nature 336: 577-580 (1988)), any sequences mapping to that area may represent associated or regulatory genes for further investigation. The nucleotide sequence of the subject invention may also be used to detect differences in the chromosomal location due to translocation, inversion, etc., among normal, carrier, or affected individuals.

It shall be understood that all of the methods (e.g., PCR and FISH) that detect polynucleotides encoding a polypeptide with ROS or aLK kinase activity, may be combined with other methods that detect polypeptides with ROS or ALK kinase activity or polynucleotides encoding a polypeptide with ROS or ALK kinase activity. For example, detection of a FIG-ROS (S) fusion polynucleotide in the genetic material of a biological sample (e.g., FIG-ROS(S) in a circulating tumor cell) may be followed by Western blotting analysis or immuno-histochemistry (IHC) analysis of the proteins of the sample to determine if the FIG-ROS(S) polynucleotide was actually expressed as a FIG-ROS(S) fusion polypeptide in the biological sample. Such Western blotting or IHC analyses may be performed using an antibody that specifically binds to the polypeptide encoded by the detected FIG-ROS(S) polynucleotide, or the analyses may be performed using antibodies that specifically bind either to full length FIG (e.g., bind to the N-terminus of the protein) or to full length ROS (e.g., bind an epitope in the kinase domain of ROS). Such assays are known in the art (see, e.g., U.S. Pat. No. 7,468,252).

In another example, the CISH technology of Dako allows chromatogenic in situ hybridization with immuno-histochemistry on the same tissue section. See Elliot et al., Br J Biomed Sci 2008; 65(4): 167-171, 2008 for a comparison of CISH and FISH.

Another aspect of the invention provides a method for diagnosing a patient as having a cancer or a suspected cancer driven by an ROS kinase or an ALK kinase. The method includes contacting a biological sample of said cancer or a suspected cancer (where the biological sample comprising at least one nucleic acid molecule) with a probe that hybridizes under stringent conditions to a nucleic acid molecule encoding a polypeptide with ROS or ALK kinase activity such as a full length ROS or ALK polynucleotide or a ROS or ALK fusion polynucleotide, and wherein hybridization of said probe to at least one nucleic acid molecule in said biological sample identifies said patient as having a cancer or a suspected Icancer driven by a ROS kinase.

Yet another aspect of the invention provides a method for diagnosing a patient as having a cancer or a suspected cancer driven by a ROS kinase or ALK kinase. The method includes contacting a biological sample of said cancer or suspected cancer (where said biological sample comprises at least one polypeptide) with a reagent that specifically binds to a polypeptide with ROS or ALK kinase activity, wherein specific binding of said reagent to at least one polypeptide in said biological sample identifies said patient as having a lung cancer or a suspected lung cancer driven by a ROS kinase or an ALK kinase.

In various embodiments, the identification of a lung cancer or suspected lung cancer as being driven by a ROS kinase or an ALK kinase will identify that patient having that lung cancer or suspected lung cancer as being likely to respond to a ROS-inhibiting therapeutic, an ALK-inhibiting therapeutic, or both (or to a ROS/ALK-inhibiting therapeutic).

In order to provide a basis for the diagnosis of disease (e.g., a lung cancer) characterized by expression of a polypeptide with ROS or ALK kinase activity, a normal or standard profile for expression may be established. This may be accomplished by combining body fluids or cell extracts taken from normal subjects, either animal or human, with a polynucleotide sequence, or a fragment thereof, which encodes a polypeptide with ROS or ALK kinase activity, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease.

Once disease is established and a treatment protocol is initiated, hybridization assays may be repeated on a regular basis to evaluate whether the level of expression in the patient begins to approximate that which is observed in the normal patient. The results obtained from successive assays may be used to show the efficacy of treatment over a period ranging from several days to months.

A similar normal or standard profile for expression or activity level of a polypeptide having ROS or ALK kinase activity can be established. For example, for protein expression, the profile can be established using a reagent that specifically binds to the polypeptide can also be established using, e.g., an antibody that specifically binds to the polypeptide (e.g., binds to full length ROS or binds to the fusion junction of a ROS fusion polypeptide) and comparing levels of binding in normal subject with levels of binding in patients symptomatic for lung cancer. Similarly, for ROS or ALK kinase activity levels, a standard in vitro kinase assay (see Ausubel et al., supra; Sambrook et al., supra) can be performed on a samples taken from normal patients as compared to samples taken from patients symptomatic for lung cancer.

In various embodiments, the inhibition of ROS or ALK expression or kinase activity is determined using a reagent that specifically binds to a ROS or ALK fusion polynucleotide, a reagent that specifically binds to ROS or ALK fusion polypeptide, a reagent that specifically binds to a full length ROS or ALK polynucleotide, or a reagent that specifically binds to a full length ROS or ALK polypeptide. In some additional embodiments, the inhibition of ROS or ALK expression or kinase activity is determined using a reagent that specifically binds to the full length protein of a fusion partner of a ROS fusion polypeptide or a ALK fusion protein. For example, for ROS, the reagent may specifically bind a FIG or CD74 or SLC34A2 polynucleotide or specifically binds to a full length FIG or CD74 or SLC34A2 polypeptide. For ROS, the reagent may specifically binds to a full length NPM or EML4 or ATIC or CARS or TFG or KIF5B or RANBP2 or TPM3, or ALO17 or MSN or TPM4 or ATIC or MYH9 or CLTC or SEC31 L1 polynucleotide, or may specifically binds to a full length NPM or EML4 or ATIC or CARS or TFG or KIF5B or RANBP2 or TPM3, or ALO17 or MSN or TPM4 or ATIC or MYH9 or CLTC or SEC31L1 polypeptide.

In various embodiments, the expression and/or activity of said polypeptide is inhibited with a composition comprising a therapeutic selected from the group consisting of crizotinib (also known as PF-02341066), NVT TAE-684, AP26113, CEP-14083, CEP-14513, CEP11988, WHI-P131 and WHI-P154.

As used herein, a “ROS inhibitor” or a “ROS-inhibiting compound” means any composition comprising one or more compounds, chemical or biological, which inhibits, either directly or indirectly, the expression and/or activity of a polypeptide with ROS kinase activity. Such inhibition may be in vitro or in vivo. “ROS inhibitor therapeutic” or “ROS-inhibiting therapeutic” means a ROS-inhibiting compound used as a therapeutic to treat a patient harboring a cancer (e.g., a lung cancer such as NSCLC or SCLC) characterized by the presence of a polypeptide with ROS kinase activity such as aberrantly expressed full length ROS protein or a ROS fusion polypeptide (e.g., one of the FIG-ROS fusion proteins) described herein.

In some embodiments of the invention, the ROS inhibitor is a reagent that specifically binds to a ROS fusion polypeptide (e.g., FIG-ROS(S), FIG-ROS(L), FIG-ROS(XL), SLC34A2-ROS(VS), SLC34A2-ROS(S), SLC34A2-ROS(L), or CD74-ROS), a reagent that specifically binds to a full length ROS polypeptide, an siRNA targeting a ROS fusion polynucleotide (e.g., an SLC34A2-ROS(S) fusion polynucleotide) or an siRNA targeting a full length ROS polynucleotide. Non-limiting siRNAs for inhibiting ROS protein expression are as follows:

(ROS1(6318-6340)
(SEQ ID NO: 13)
5′AAGCCCGGAUGGCAACGUUTT3′;
or
(ROS1(7181-7203)
(SEQ ID NO: 14)
5′AAGCCUGAAGGCCUGAACUTT3′.

The ability of these two siRNAs to inhibit ROS kinase activity has been described (see U.S. Patent Publication No. 20100221737, incorporated by reference.

As used herein, a “ALK inhibitor” or a “ALK-inhibiting compound” means any composition comprising one or more compounds, chemical or biological, which inhibits, either directly or indirectly, the expression and/or activity of a polypeptide with ALK kinase activity. Such inhibition may be in vitro or in vivo. “ALK inhibitor therapeutic” or “ALK-inhibiting therapeutic” means a ALK-inhibiting compound used as a therapeutic to treat a patient harboring a cancer (e.g., a lung cancer such as NSCLC or SCLC) characterized by the presence of a polypeptide with ALK kinase activity such as aberrantly expressed full length ALK protein or a ALK fusion polypeptide (e.g., one of the EM4-ALK fusion proteins) described herein.

The ALK and/or ROS-inhibiting therapeutic may be, for example, a kinase inhibitor, such as a small molecule or antibody inhibitor. It may be a pan-kinase inhibitor with activity against several different kinases, or a kinase-specific inhibitor. Since ROS, ALK, LTK, InsR, and IGFIR belong to the same family of tyrosine kinases, they may share similar structure in the kinase domain. Thus, in some embodiments, an ALK and/or ROS inhibitor of the invention also inhibits the activity of an ALK kinase, an LTK kinase, an insulin receptor, or an IGF1 receptor. ROS-inhibiting compounds are discussed in further detail below. Patient biological samples may be taken before and after treatment with the inhibitor and then analyzed, using methods described above, for the biological effect of the inhibitor on ALK or ROS kinase activity, including the phosphorylation of downstream substrate protein. Such a pharmacodynamic assay may be useful in determining the biologically active dose of the drug that may be preferable to a maximal tolerable dose. Such information would also be useful in submissions for drug approval by demonstrating the mechanism of drug action.

In another embodiment, the expression and/or activity of said polypeptide is inhibited with a composition comprising a ROS or ALK inhibiting therapeutic selected from the group consisting of PF-02341066), NVT TAE-684, AP26113, CEP-14083, CEP-14513, CEP11988, WHI-P131 and WHI-P154.

In accordance with the present invention, the polypeptide with ROS or ALK kinase activity may occur in at least one subgroup of human cancer. Accordingly, the progression of a mammalian cancer in which a polypeptide with ROS or ALK kinase activity is expressed may be inhibited, in vivo, by inhibiting the activity of ROS or ALK kinase in such cancer. ROS or ALK activity in cancers characterized by expression of a polypeptide with ROS or ALK kinase activity may be inhibited by contacting the cancer with a therapeutically effective amount of a ROS-inhibiting and/or ALK-inhibiting therapeutic. Accordingly, the invention provides, in part, a method for inhibiting the progression of polypeptide with ROS or ALK kinase activity-expressing lung cancer by inhibiting the expression and/or activity of ROS or ALK kinase in the lung cancer by contacting the cancer (e.g., a tumor) with a therapeutically effective amount of an ROS-inhibiting therapeutic and/or ALK-inhibiting therapeutic.

As used herein, by “therapeutically effective amount” or “pharmaceutically effective amount” is mean an amount of an ROS-inhibiting therapeutic and/or ALK-inhibiting therapeutic that is adequate to inhibit the cancer (or cell thereof) or suspected cancer (or cells thereof), as compared to an untreated cancer or suspected cancer, by either slowing the growth of the cancer or suspected cancer, reducing the mass of the cancer or suspected cancer, reducing the number of cells of the cancer or suspected cancer, or killing the cancer.

A ROS-inhibiting therapeutic and/or ALK-inhibiting therapeutic may be any composition comprising at least one ROS or ALK inhibitor. Such compositions also include compositions comprising only a single ROS- or ALK-inhibiting compound, as well as compositions comprising multiple therapeutics (including those against other RTKs), which may also include a non-specific therapeutic agent like a chemotherapeutic agent or general transcription inhibitor.

In some embodiments, a ROS-inhibiting therapeutic and/or ALK-inhibiting therapeutic useful in the practice of the methods of the invention is a targeted, small molecule inhibitor. Small molecule targeted inhibitors are a class of molecules that typically inhibit the activity of their target enzyme by specifically, and often irreversibly, binding to the catalytic site of the enzyme, and/or binding to an ATP-binding cleft or other binding site within the enzyme that prevents the enzyme from adopting a conformation necessary for its activity. Because of the close similarity in structure and function between the ROS kinase and the ALK kinase, any ALK kinase inhibitor is predicted to also inhibit ROS kinase. Additionally, as described below in the examples, a lung cancer driven by ROS kinase will not driven by ALK kinase. Likewise, a lung cancer driven by ALK kinase will not be driven by ROS kinase.

Accordingly, in another aspect, the invention provides a method of treating a patient for lung cancer, comprising: detecting the presence in a biological sample from a lung of a patient having or suspected of having lung cancer of a polypeptide selected from the group consisting of a polypeptide having ROS kinase activity and a polypeptide having ALK kinase activity; and administering an effective amount of an ALK/ROS-inhibiting therapeutic to the patient, thereby treating the subject for lung cancer.

In a further aspect, the invention provides a method for identifying a patient with lung cancer or suspected of having lung cancer as a patient likely to respond to a ROS-inhibiting therapeutic, comprising: contacting a biological sample from a lung of said patient with a first reagent that specifically binds a polypeptide having ROS kinase activity and a second reagent that specifically binds to a polypeptide having ALK knase activity and detecting whether the first reagent or the second reagent specifically binds to the biological sample, wherein detection of binding of either the first reagent or the second reagent to the biological sample identifies the patient as a patient likely to respond to a ROS-inhibiting therapeutic. In various embodiments, the first reagent specifically binds to full length ROS kinase protein. In various embodiments, the second reagent specifically binds to full length ALK kinase protein. In various embodiments, the first reagent specifically binds to the kinase domain of ROS kinase protein. In various embodiments, the second reagent specifically binds to the kinase domain of ALK kinase protein.

As used herein, by “protein having ALK kinase activity” is meant any polypeptide that retains the full kinase domain of ALK and thus, has ALK kinase activity. Non-limiting polypeptides with ALK kinase activity include full length ALK (see U.S. Pat. No. 5,770,421), NPM-ALK, ALO17-ALK, TFG-ALK, MSN-ALK, TPM3-ALK, TPM4-ALK, ATIC-ALK, MYH9-ALK, CLTC-ALK, SEC31L1-ALK, RANBP2-ALK, CARS-ALK, EML4-ALK, KIF5B-ALK, and TFG-ALK (see, e.g., Palmer et al., Biochem. J. 420(3): 345-361, 2009 (and the articles cited therein), Rikova et al., Cell 131: 1190-1203, 2007; Soda et al., Nature 448: 561-566, 2007; Morris et al., Science 263: 1281-1284, 1994; Du et al., J. Mol. Med 84: 863-875, 2007; Panagopoulos et al., Int. J. Cancer 118: 1181-1186, 2006; Cools et al., Genes Chromosomes Cancer 34: 354-362, 2002; Debelenko et al., Lab. Invest. 83: 1255-1265, 2003; Ma et al., Genes Chromosomes Cancer 37: 98-105, 2003; Lawrence et al., Am. J. Pathol. 157: 377-384, 1995; Hernandez et al., Blood 94: 3265-3268, 1999; Takeuchi K., Clin Cancer Res. 15(9):3143-3149, 2009; Tort et al., Lab. Invest. 81: 419-426, 2001; Trinei et al., Cancer Res. 60: 793-798, 2000; and Touriol et al., Blood 95: 3204-3207, 2000. See also Pulford et al., Journal of Cellular Physiology, 199:330-358, 2004.

In various embodiments, the patient is a human. In various embodiments, the lung cancer is non-small cell lung cancer or is small cell lung cancer.

One useful small-molecule kinase inhibitor is Pfizer, Inc.'s compound Crizotinib (also known as PF-02341066), which inhibits ALK and MET kinase activity, and its properties have been well described. See You et al., Cancer Res 67: 4408 (2007) and U.S. Patent Pub. No. 2008/0300273. Additional small molecule kinase inhibitors that may target ROS include TAE-684 (from Novartis), CH5424802 (Chugai; see Sakamoto, H. et al., Cancer Cell 19: 679-690, 2011), AP26113 (Ariad Pharmaceuticals, Inc.), and CEP-14083, CEP-14513, and CEP-11988 (Cephalon; see Wan et al., Blood 107: 1617-1623, 2006).

PF-02341066 has the structure:

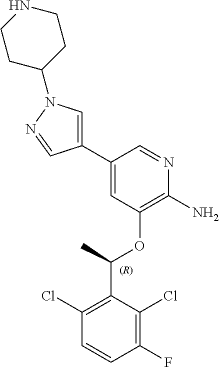

TAE-684, a 5-chloro-2,4-diaminophenylpyrimidine, which has the structure:

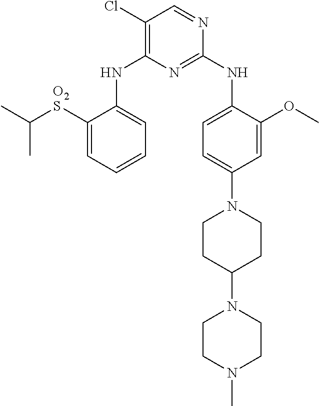 [SMILES: COc1cc(N2CCC(N3CCN(C)CC3)CC2)ccc1Nc1ncc(Cl)c(Nc2ccccc2S(=O)(=O)C(C)C)n1]

and has been shown to inhibit the ROSA LK kinase. Grosin, et al., Proc. National Acad. Sci 104(1) 270-275, 2007.

Additional small molecule inhibitors and other inhibitors (e.g., indirect inhibitors) of ROS kinase activity may be rationally designed using X-ray crystallographic or computer modeling of ROS three dimensional structure, or may found by high throughput screening of compound libraries for inhibition of key upstream regulatory enzymes and/or necessary binding molecules, which results in inhibition of ROS or ALK kinase activity. Such approaches are well known in the art, and have been described. ROS inhibition or ALK inhibition by such therapeutics may be confirmed, for example, by examining the ability of the compound to inhibit ROS or ALK kinase activity, but not other kinase activity, in a panel of kinases, and/or by examining the inhibition of ROS or ALK activity in a biological sample comprising cancer cells (e.g., lung cancer cells). Methods for identifying compounds that inhibit a cancer characterized by the expression/presence of polypeptide with ROS or ALK kinase activity, are further described below.

ROS-inhibiting therapeutics and/or ALK-inhibiting therapeutic useful in the methods of the invention may also be targeted antibodies that specifically bind to critical catalytic or binding sites or domains required for ROS or ALK activity, and inhibit the kinase by blocking access of ligands, substrates or secondary molecules to at and/or preventing the enzyme from adopting a conformation necessary for its activity. The production, screening, and therapeutic use of humanized target-specific antibodies has been well-described. See Merluzzi et al., Adv Clin Path. 4(2): 77-85 (2000). Commercial technologies and systems, such as Morphosys, Inc.'s Human Combinatorial Antibody Library (HuCAL®), for the high-throughput generation and screening of humanized target-specific inhibiting antibodies are available.

The production of various anti-receptor kinase targeted antibodies and their use to inhibit activity of the targeted receptor has been described. See, e.g. U.S. Patent Publication No. 20040202655, U.S. Patent Publication No. 20040086503, U.S. Patent Publication No. 20040033543, Standardized methods for producing, and using, receptor tyrosine kinase activity-inhibiting antibodies are known in the art. See, e.g., European Patent No. EP1423428,

Phage display approaches may also be employed to generate ROS-specific or ALK-specific antibody inhibitors, and protocols for bacteriophage library construction and selection of recombinant antibodies are provided in the well-known reference text CURRENT PROTOCOLS IN IMMUNOLOGY, Colligan et al. (Eds.), John Wiley & Sons, Inc. (1992-2000), Chapter 17, Section 17.1. See also U.S. Pat. No. 6,319,690, U.S. Pat. No. 6,300,064, U.S. Pat. No. 5,840,479, and U.S. Patent Publication No. 20030219839.

A library of antibody fragments displayed on the surface of bacteriophages may be produced (see, e.g. U.S. Pat. No. 6,300,064) and screened for binding to a polypeptide with ROS kinase activity or ALK kinase activity. See European Patent No. EP1423428.

Antibodies identified in screening of antibody libraries as described above may then be further screened for their ability to block the activity of ROS or ALK, both in vitro kinase assay and in vivo in cell lines and/or tumors. ROS or ALK inhibition may be confirmed, for example, by examining the ability of such antibody therapeutic to inhibit ROS or ALK kinase activity in a panel of kinases, and/or by examining the inhibition of ROS or ALK activity in a biological sample comprising cancer cells, as described above. In some embodiments, a ROS-inhibiting compound of the invention reduces ROS kinase activity, but reduces the kinase activity of other kinases to a lesser extent (or not at all). Likewise, in some embodiments, an ALK-inhibiting compound of the invention reduces ALK kinase activity, but reduces the kinase activity of other kinases to a lesser extent (or not at all). Methods for screening such compounds for ROS and/or ALK kinase inhibition are further described above.

ROS-inhibiting or ALK-inhibiting compounds that useful in the practice of the disclosed methods may also be compounds that indirectly inhibit ROS or ALK activity by inhibiting the activity of proteins or molecules other than ROS or ALK kinase itself. Such inhibiting therapeutics may be targeted inhibitors that modulate the activity of key regulatory kinases that phosphorylate or de-phosphorylate (and hence activate or deactivate) ROS or ALK itself, or interfere with binding of ligands. As with other receptor tyrosine kinases, both ROS and ALK regulates downstream signaling through a network of adaptor proteins and downstream kinases. As a result, induction of cell growth and survival by ROS or ALK activity may be inhibited by targeting these interacting or downstream proteins.

ROS or ALK kinase activity may also be indirectly inhibited by using a compound that inhibits the binding of an activating molecule necessary for these full length and fusion polypeptide (e.g., an CD74-ROS or an EML4-ALK fusion polypeptide) to adopt its active conformation (i.e., such that the kinase domain is able to be activated). For example, the production and use of anti-PDGF antibodies has been described. See U.S. Patent Publication No. 20030219839, “Anti-PDGF Antibodies and Methods for Producing Engineered Antibodies,” Bowdish et al. Inhibition of ligand (PDGF) binding to the receptor directly down-regulates the receptor activity.

ROS and/or ALK inhibiting compounds or therapeutics may also comprise anti-sense and/or transcription inhibiting compounds that inhibit ROS or ALk kinase activity by blocking transcription of the gene encoding polypeptides with ROS or ALK kinase activity. The inhibition of various receptor kinases, including VEGFR, EGFR, and IGFR, and FGFR, by antisense therapeutics for the treatment of cancer has been described. See, e.g., U.S. Pat. Nos. 6,734,017; 6,710,174, 6,617,162; 6,340,674; 5,783,683; 5,610,288.

Antisense oligonucleotides may be designed, constructed, and employed as therapeutic agents against target genes in accordance with known techniques. See, e.g. Cohen, J., Trends in Pharmacol. Sci. 10(11): 435-437 (1989); Marcus-Sekura, Anal. Biochem. 172: 289-295 (1988); Weintraub, H., Sci. AM. Pp. 40-46 (1990); Van Der Krol et al., BioTechniques 6(10): 958-976 (1988); Skorski et al., Proc. Natl. Acad. Sci. USA (1994) 91: 4504-4508. Inhibition of human carcinoma growth in vivo using an antisense RNA inhibitor of EGFR has recently been described. See U.S. Patent Publication No. 20040047847. Similarly, a ROS-inhibiting or ALK-inhibiting therapeutic comprising at least one antisense oligonucleotide against a mammalian ROS or ALK gene or a mammalian ROS or ALK fusion protein-encoding polynucleotide may be prepared according to standard methods. Pharmaceutical compositions comprising ROS-inhibiting antisense compounds may be prepared and administered as further described below.

Small interfering RNA molecule (siRNA) compositions, which inhibit translation, and hence activity, of ROS or ALK through the process of RNA interference, may also be desirably employed in the methods of the invention. RNA interference, and the selective silencing of target protein expression by introduction of exogenous small double-stranded RNA molecules comprising sequence complimentary to mRNA encoding the target protein, has been well described. See, e.g. U.S. Patent Publication No. 20040038921, U.S. Patent Publication No. 20020086356, and U.S. Patent Publication 20040229266.

Double-stranded RNA molecules (dsRNA) have been shown to block gene expression in a highly conserved regulatory mechanism known as RNA interference (RNAi). Briefly, the RNAse III Dicer processes dsRNA into small interfering RNAs (siRNA) of approximately 22 nucleotides, which serve as guide sequences to induce target-specific mRNA cleavage by an RNA-induced silencing complex RISC (see Hammond et al., Nature (2000) 404: 293-296). RNAi involves a catalytic-type reaction whereby new siRNAs are generated through successive cleavage of longer dsRNA. Thus, unlike antisense, RNAi degrades target RNA in a non-stoichiometric manner. When administered to a cell or organism, exogenous dsRNA has been shown to direct the sequence-specific degradation of endogenous messenger RNA (mRNA) through RNAi.

A wide variety of target-specific siRNA products, including vectors and systems for their expression and use in mammalian cells, are now commercially available. See, e.g., Promega, Inc. (www.promega.com); Dharmacon, Inc. (www.dharmacon.com). Detailed technical manuals on the design, construction, and use of dsRNA for RNAi are available. See, e.g., Dharmacon's “RNAi Technical Reference & Application Guide”; Promega's “RNAi: A Guide to Gene Silencing.” ROS-inhibiting siRNA products are also commercially available, and may be suitably employed in the method of the invention. See, e.g., Dharmacon, Inc., Lafayette, Colo. (Cat Nos. M-003162-03, MU-003162-03, D-003162-07 thru-10 (siGENOME™ SMARTselection and SMARTpool® siRNAs).

It has recently been established that small dsRNA less than 49 nucleotides in length, and preferably 19-25 nucleotides, comprising at least one sequence that is substantially identical to part of a target mRNA sequence, and which dsRNA optimally has at least one overhang of 1-4 nucleotides at an end, are most effective in mediating RNAi in mammals. See U.S. Patent Publication Nos. 20040038921 and 20040229266. The construction of such dsRNA, and their use in pharmaceutical preparations to silence expression of a target protein, in vivo, are described in detail in such publications.

If the sequence of the gene to be targeted in a mammal is known, 21-23 nt RNAs, for example, can be produced and tested for their ability to mediate RNAi in a mammalian cell, such as a human or other primate cell. Those 21-23 nt RNA molecules shown to mediate RNAi can be tested, if desired, in an appropriate animal model to further assess their in vivo effectiveness. Target sites that are known, for example target sites determined to be effective target sites based on studies with other nucleic acid molecules, for example ribozymes or antisense, or those targets known to be associated with a disease or condition such as those sites containing mutations or deletions, can be used to design siRNA molecules targeting those sites as well.

Alternatively, the sequences of effective dsRNA can be rationally designed/predicted screening the target mRNA of interest for target sites, for example by using a computer folding algorithm. The target sequence can be parsed in silico into a list of all fragments or subsequences of a particular length, for example 23 nucleotide fragments, using a custom Perl script or commercial sequence analysis programs such as Oligo, MacVector, or the GCG Wisconsin Package.

Various parameters can be used to determine which sites are the most suitable target sites within the target RNA sequence. These parameters include but are not limited to secondary or tertiary RNA structure, the nucleotide base composition of the target sequence, the degree of homology between various regions of the target sequence, or the relative position of the target sequence within the RNA transcript. Based on these determinations, any number of target sites within the RNA transcript can be chosen to screen siRNA molecules for efficacy, for example by using in vitro RNA cleavage assays, cell culture, or animal models. See, e.g., U.S. Patent Publication No. 20030170891. An algorithm for identifying and selecting RNAi target sites has also recently been described. See U.S. Patent Publication No. 20040236517.

Commonly used gene transfer techniques include calcium phosphate, DEAE-dextran, electroporation and microinjection and viral methods (Graham et al. (1973) Virol. 52: 456; McCutchan et al., (1968), J. Natl. Cancer Inst. 41: 351; Chu et al. (1987), Nucl. Acids Res. 15: 1311; Fraley et al. (1980), J. Biol. Chem. 255: 10431; Capecchi (1980), Cell 22: 479). DNA may also be introduced into cells using cationic liposomes (Feigner et al. (1987), Proc. Natl. Acad. Sci USA 84: 7413). Commercially available cationic lipid formulations include Tfx 50 (Promega Corp., Fitchburg, Wis.) or Lipofectamin 200 (Life Technologies, Carlsbad, Calif.). Alternatively, viral vectors may be employed to deliver dsRNA to a cell and mediate RNAi. See U.S Patent Publication No. 20040023390.

Transfection and vector/expression systems for RNAi in mammalian cells are commercially available and have been well described. See, e.g., Dharmacon, Inc. (Lafayette, Colo.), DharmaFECT™ system; Promega, Inc., siSTRIKE™ U6 Hairpin system; see also Gou et al. (2003) FEBS. 548, 113-118; Sui, G. et al. A DNA vector-based RNAi technology to suppress gene expression in mammalian cells (2002) Proc. Natl. Acad. Sci. 99, 5515-5520; Yu et al. (2002) Proc. Natl. Acad. Sci. 99, 6047-6052; Paul, C. et al. (2002) Nature Biotechnology 19, 505-508; McManus et al. (2002) RNA 8, 842-850.

siRNA interference in a mammal using prepared dsRNA molecules may then be effected by administering a pharmaceutical preparation comprising the dsRNA to the mammal. The pharmaceutical composition is administered in a dosage sufficient to inhibit expression of the target gene. dsRNA can typically be administered at a dosage of less than 5 mg dsRNA per kilogram body weight per day, and is sufficient to inhibit or completely suppress expression of the target gene. In general a suitable dose of dsRNA will be in the range of 0.01 to 2.5 milligrams per kilogram body weight of the recipient per day, preferably in the range of 0.1 to 200 micrograms per kilogram body weight per day, more preferably in the range of 0.1 to 100 micrograms per kilogram body weight per day, even more preferably in the range of 1.0 to 50 micrograms per kilogram body weight per day, and most preferably in the range of 1.0 to 25 micrograms per kilogram body weight per day. A pharmaceutical composition comprising the dsRNA is administered once daily, or in multiple sub-doses, for example, using sustained release formulations well known in the art. The preparation and administration of such pharmaceutical compositions may be carried out accordingly to standard techniques, as further described below.

Such dsRNA may then be used to inhibit ROS expression and activity in a cancer, by preparing a pharmaceutical preparation comprising a therapeutically-effective amount of such dsRNA, as described above, and administering the preparation to a human subject having a lung cancer or suspected lung cancer (e.g., a NSCLC or SCLC) expressing a polypeptide with ROS or ALK kinase activity (such as, for example, aberrant expression of full length ROS or ALK protein or expression of a ROS or ALK fusion protein), for example, via direct injection to the tumor. The similar inhibition of other receptor tyrosine kinases, such as VEGFR and EGFR using siRNA inhibitors has recently been described. See U.S. Patent Publication No. 20040209832, U.S. Patent Publication No. 20030170891, and U.S. Patent Publication No. 20040175703.

ROS-inhibiting and/or ALK-inhibiting therapeutics useful in the practice of the methods of the invention may be administered to a mammal by any means known in the art including, but not limited to oral or peritoneal routes, including intravenous, intramuscular, intraperitoneal, subcutaneous, transdermal, airway (aerosol), rectal, vaginal and topical (including buccal and sublingual) administration.

For oral administration, a ROS-inhibiting and/or ALK-inhibiting therapeutic will generally be provided in the form of tablets or capsules, as a powder or granules, or as an aqueous solution or suspension. Tablets for oral use may include the active ingredients mixed with pharmaceutically acceptable carriers and excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatin capsules in which the active ingredient is mixed with a solid diluent, and soft gelatin capsules wherein the active ingredients is mixed with water or an oil such as peanut oil, liquid paraffin or olive oil. For intramuscular, intraperitoneal, subcutaneous and intravenous use, the pharmaceutical compositions of the invention will generally be provided in sterile aqueous solutions or suspensions, buffered to an appropriate pH and isotonicity. Suitable aqueous vehicles include Ringer's solution and isotonic sodium chloride. The carrier may consist exclusively of an aqueous buffer (“exclusively” means no auxiliary agents or encapsulating substances are present which might affect or mediate uptake of the ROS- and/or ALK-inhibiting therapeutic). Such substances include, for example, micellar structures, such as liposomes or capsids, as described below. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

ROS-inhibiting and/or ALK-inhibiting therapeutic compositions may also include encapsulated formulations to protect the therapeutic (e.g., a dsRNA compound or an antibody that specifically binds a ROS or ALK fusion polypeptide) against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811; PCT publication WO 91/06309; and European patent publication EP-A-43075. An encapsulated formulation may comprise a viral coat protein. The viral coat protein may be derived from or associated with a virus, such as a polyoma virus, or it may be partially or entirely artificial. For example, the coat protein may be a Virus Protein 1 and/or Virus Protein 2 of the polyoma virus, or a derivative thereof.

ROS-inhibiting and/or ALK-inhibiting therapeutics can also comprise a delivery vehicle, including liposomes, for administration to a subject, carriers and diluents and their salts, and/or can be present in pharmaceutically acceptable formulations. For example, methods for the delivery of nucleic acid molecules are described in Akhtar et al., 1992, Trends Cell Bio., 2, 139; DELIVERY STRATEGIES FOR ANTISENSE OLIGONUCLEOTIDE THERAPEUTICS, ed. Akbtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192. U.S. Pat. No. 6,395,713 and PCT Publication No. WO 94/02595 further describe the general methods for delivery of nucleic acid molecules. These protocols can be utilized for the delivery of virtually any nucleic acid molecule.

ROS-inhibiting and/or ALK-inhibiting therapeutics (i.e., a ROS- or ALK-inhibiting compound being administered as a therapeutic) can be administered to a mammalian tumor by a variety of methods known to those of skill in the art, including, but not restricted to, encapsulation in liposomes, by iontophoresis, or by incorporation into other vehicles, such as hydrogels, cyclodextrins, biodegradable nanocapsules, and bioadhesive microspheres, or by proteinaceous vectors (see PCT Publication No. WO 00/53722). Alternatively, the therapeutic/vehicle combination is locally delivered by direct injection or by use of an infusion pump. Direct injection of the composition, whether subcutaneous, intramuscular, or intradermal, can take place using standard needle and syringe methodologies, or by needle-free technologies such as those described in Conry et al., 1999, Clin. Cancer Res., 5, 2330-2337 and PCT Publication No. WO 99/3 1262.

Pharmaceutically acceptable formulations of ROS-inhibiting and/or ALK-inhibiting therapeutics include salts of the above described compounds, e.g., acid addition salts, for example, salts of hydrochloric, hydrobromic, acetic acid, and benzene sulfonic acid. A pharmacological composition or formulation refers to a composition or formulation in a form suitable for administration, e.g., systemic administration, into a cell or patient, including for example a human. Suitable forms, in part, depend upon the use or the route of entry, for example oral, transdermal, or by injection. Such forms should not prevent the composition or formulation from reaching a target cell. For example, pharmacological compositions injected into the blood stream should be soluble. Other factors are known in the art, and include considerations such as toxicity and forms that prevent the composition or formulation from exerting its effect.

Administration routes that lead to systemic absorption (e.g., systemic absorption or accumulation of drugs in the blood stream followed by distribution throughout the entire body), are desirable and include, without limitation: intravenous, subcutaneous, intraperitoneal, inhalation, oral, intrapulmonary and intramuscular. Each of these administration routes exposes the ROS-inhibiting therapeutic to an accessible diseased tissue or tumor. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By “pharmaceutically acceptable formulation” is meant, a composition or formulation that allows for the effective distribution of the nucleic acid molecules of the instant invention in the physical location most suitable for their desired activity. Nonlimiting examples of agents suitable for formulation with the nucleic acid molecules of the instant invention include: P-glycoprotein inhibitors (such as Pluronic P85), which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich et al, 1999, Cell Transplant, 8, 47-58) (Rosermes, Inc. Cambridge, Mass.); and loaded nanoparticles, such as those made of polybutylcyanoacrylate, which can deliver drugs across the blood brain barrier and can alter neuronal uptake mechanisms (Prog Neuro-psychopharmacol Biol Psychiatry, 23, 941-949, 1999). Other non-limiting examples of delivery strategies for the ROS-inhibiting compounds useful in the method of the invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

Therapeutic compositions comprising surface-modified liposomes containing poly (ethylene glycol) lipids (PEG-modified, or long-circulating liposomes or stealth liposomes) may also be suitably employed in the methods of the invention. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; PCT Publication No. WO 96/10391; PCT Publication No. WO 96/10390; and PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

Therapeutic compositions may include a pharmaceutically effective amount of the desired compounds in a pharmaceutically acceptable carrier or diluent. Acceptable carriers or diluents for therapeutic use are well known in the pharmaceutical art, and are described, for example, in REMINGTON'S PHARMACEUTICAL SCIENCES, Mack Publishing Co. (A. R. Gennaro edit. 1985). For example, preservatives, stabilizers, dyes and flavoring agents can be provided. These include sodium benzoate, sorbic acid and esters of p-hydroxybenzoic acid. In addition, antioxidants and suspending agents can be used.

In some embodiments, the ROS-inhibiting therapeutic and/or the ALK-inhibiting therapeutic is administered in an effective amount. By “effective amount” or “effective dose” is meant the amount of the therapeutic required to prevent, inhibit the occurrence, or treat (alleviate a symptom to some extent, preferably all of the symptoms) of a disease state (e.g., lung cancer). The effective dose depends on the type of disease, the therapeutic used, the route of administration, the type of mammal being treated, the physical characteristics of the specific mammal under consideration, concurrent medication, and other factors that those skilled in the medical arts will recognize. Generally, an effective amount is an amount between 0.1 mg/kg and 100 mg/kg body weight/day of active ingredients is administered dependent upon potency of the negatively charged polymer.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient. It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

A ROS-inhibiting and/or ALK-inhibiting therapeutic useful in the practice of the invention may comprise a single compound as described above, or a combination of multiple compounds, whether in the same class of inhibitor (e.g., antibody inhibitor), or in different classes (e.g., antibody inhibitors and small-molecule inhibitors). Such combination of compounds may increase the overall therapeutic effect in inhibiting the progression of a fusion protein-expressing cancer. For example, the therapeutic composition may a small molecule inhibitor, such as Crizotinib (also known as PF-02341066) produced by Pfizer, Inc. (see U.S. Pub. No. 2008/0300273) alone, or in combination with other Crizotinib analogues targeting ROS activity and/or small molecule inhibitors of ROS, such as NVP-TAE684 produced by Novartis, Inc., or the CH5424802 compound described in Sakamoto et al., Cancer Cell 19: 679-690, 2011. The therapeutic composition may also comprise one or more non-specific chemotherapeutic agent in addition to one or more targeted inhibitors. Such combinations have recently been shown to provide a synergistic tumor killing effect in many cancers. The effectiveness of such combinations in inhibiting ROS activity and tumor growth in vivo can be assessed as described below.

The invention also provides, in part, a method for determining whether a compound inhibits the progression of a cancer (e.g., a lung cancer) characterized by a polypeptide with ROS or ALK kinase activity or polynucleotide encoding the same by determining whether the compound inhibits the ROS or ALK kinase activity of the polypeptide in the cancer. In some embodiments, inhibition of activity of ROS or ALK is determined by examining a biological sample comprising cells from bone marrow, blood, or a tumor. In another embodiment, inhibition of activity of ROS or ALK kinase is determined using at least reagent that specifically binds to a ROS or ALK polypeptide (e.g., a ROS-specific antibody or an ALK-specific antobpdu) or a reagent that specifically binds to a ROS or ALK polypeptide-encoding polynucleotide (e.g., an siRNA or an antisense).

The tested compound may be any type of therapeutic or composition as described above. Methods for assessing the efficacy of a compound, both in vitro and in vivo, are well established and known in the art. For example, a composition may be tested for ability to inhibit ROS in vitro using a cell or cell extract in which ROS kinase is activated. A panel of compounds may be employed to test the specificity of the compound for ROS (as opposed to other targets, such as PDGFR). For example, a composition may be tested for ability to inhibit ALK kinase activity in vitro using a cell or cell extract in which ALK kinase is activated.

Another technique for drug screening which may be used provides for high throughput screening of compounds having suitable binding affinity to a protein of interest, as described in PCT Publication No. WO 84/03564. In this method, as applied to polypeptides having ROS or ALK activity, large numbers of different small test compounds are synthesized on a solid substrate, such as plastic pins or some other surface. The test compounds are reacted with a polypeptide of the invention, or fragments thereof, and washed. Bound polypeptide is then detected by methods well known in the art. A purified polypeptide can also be coated directly onto plates for use in the aforementioned drug screening techniques. Alternatively, non-neutralizing antibodies can be used to capture the peptide and immobilize it on a solid support.

A compound found to be an effective inhibitor of ROS activity in vitro may then be examined for its ability to inhibit the progression of a cancer expressing a polypeptide with kinase activity (such as lung cancer or other cancer such as a liver cancer, lung cancer, colon cancer, kidney cancer, or a pancreatic cancer), in vivo, using, for example, mammalian xenografts harboring human lung, liver, pancreatic, kidney, lung, or colon tumors that are express a polypeptide with ROS or ALK kinase activity. In this procedure, cancer cell lines known to express a protein having ROS or ALK kinase activity (e.g., full length ROS or ALK or one of the ROS or ALK fusion proteins) may be placed subcutaneously in an animal (e.g., into a nude or SCID mouse, or other immune-compromised animal). The cells then grow into a tumor mass that may be visually monitored. The animal may then be treated with the drug. The effect of the drug treatment on tumor size may be externally observed. The animal is then sacrificed and the tumor removed for analysis by IHC and Western blot. Similarly, mammalian bone marrow transplants may be prepared, by standard methods, to examine drug response in hematological tumors expressing a protein with ROS or ALK kinase activity. In this way, the effects of the drug may be observed in a biological setting most closely resembling a patient. The drug's ability to alter signaling in the tumor cells or surrounding stromal cells may be determined by analysis with phosphorylation-specific antibodies. The drug's effectiveness in inducing cell death or inhibition of cell proliferation may also be observed by analysis with apoptosis specific markers such as cleaved caspase 3 and cleaved PARP.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In some embodiments, the compounds exhibit high therapeutic indices.

In practicing the disclosed method for determining whether a compound inhibits progression of a tumor characterized by the presence of a polypeptide with ROS kinase activity (or polynucleotide encoding the same), biological samples comprising cells from mammalian xenografts (or bone marrow transplants) may also be advantageously employed. Non-limiting xenografts (or transplant recipients) are small mammals, such as mice, harboring human tumors (or leukemias) that express a polypeptide with ROS or ALK kinase activity (e.g., a ROS or ALK fusion polypeptide or full length ROS or ALK). Xenografls harboring human tumors are well known in the art (see Kal, Cancer Treat Res. 72: 155-69 (1995)) and the production of mammalian xenografts harboring human tumors is well described (see Winograd et al., In Vivo. 1(1): 1-13 (1987)). Similarly the generation and use of bone marrow transplant models is well described (see, e.g., Schwaller, et al., EMBO J. 17: 5321-333 (1998); Kelly et al., Blood 99: 310-318 (2002)).

The following Examples are provided only to further illustrate the invention, and are not intended to limit its scope, except as provided in the claims appended hereto. The present invention encompasses modifications and variations of the methods taught herein which would be obvious to one of ordinary skill in the art. Materials, reagents and the like to which reference is made are obtainable from commercial sources, unless otherwise noted.

### Example 1

### Detection of ROS Kinase Protein by Immunohistochemistry (IHC)

ROS fusion proteins have previously been described in NSCLC cell lines and NSCLC human tumor samples (as well as in other tissues such as liver cancer and brain cancer). To determine whether or not the ROS fusion proteins discovered in NSCLC could be detected by immunohistochemistry, a ROS-specific rabbit monoclonal antibody was used. The ROS-specific antibody (namely rabbit monoclonal antibody ROS1 D4D6) that was used in these studies has been described previously (see PCT Publication No. WO2010/093928), and specifically binds a region on the human ROS kinase protein that is C-terminal to the kinase domain of the ROS protein. While the D4D6 antibody is not yet commercially available, similar ROS-specific antibodies are commercially available from a variety of suppliers including, without limitation, the Ros (C-20) antibody, Catalog No. sc-6347 from Santa Cruz Biotechnology, Inc., (Santa Cruz, Calif.) and the ROS (69D6) antibody, Catalog No #3266 from Cell Signaling Technology, Inc. (Danvers, Mass.).

For these studies, a cohort of 556 human samples of NSCLC tumors were prepared as paraffin blocks. All tumor samples were evaluated by two independent pathologists, and were found to comprise 246 adenocarcinoma, 64 bronchioaveolar carcinoma, 226 squamous and 20 large cell carcinoma cases.

Immunohistochemistry: 4-6 μm tissue sections were deparaffinized and rehydrated through xylene and graded ethanol, respectively (e.g., through three changes of xylene for minutes each, then rehydrated through two changes of 100% ethanol and 2 changes of 95% ethanol, each for 5 minutes). Slides were rinsed in diH₂O, then subjected to antigen retrieval in a Decloaking Chamber (Biocare Medical, Concord, Calif.) using 1.0 mM EDTA, pH 8.0 and manufacturer's settings: SP1 125° C. for 30 seconds and SP2 90° C. for 10 seconds. Slides were quenched in 3% H₂O₂ for 10 minutes, then washed in diH₂O. After blocking in Tris buffered saline positive 0.5% Tween-20 (TBST)/5% goat serum in a humidified chamber, slides were incubated overnight at 4° C. with ROS1 (D4D6) XP™ Rabbit mAb at 0.19 μg/ml diluted in SignalStain® Antibody Diluent (#8112 Cell Signaling Technology, Danvers, Mass.). After washing with TBST, detection was performed with either Envisionpositive (Dako, Carpinteria, Calif.) or SignalStain® Boost IHC Detection Reagent (HRP, Rabbit) (catalog #8114 Cell Signaling Technology, Danvers, Mass.) with a 30 minute incubation at room temperature in a humidified chamber. For the SignalStain® Boost IHC slides, After washing the slides (e.g., three times in TBST) the slides were next exposed to NovaRed (Vector Laboratories, Burlingame, Calif.) prepared per the manufacturer's instructions.

Slides were developed for 1 minute and then rinsed in diH₂O. Slides were counterstained by incubating in hematoxylin (ready to use commercially available from Invitrogen (Carlsbad, Calif.) Catalog #00-8011) for 1 minute, rinsed for 30 seconds in diH₂O, incubated for 20 seconds in bluing reagent (Richard Allan Scientific, Kalamazoo, Mich. (a Thermo Scientific company), Catalog #7301), and then finally washed for 30 seconds in diH₂O. Slides were dehydrated in 2 changes of 95% ethanol for 20 seconds each and 2 changes of 100% ethanol for 2 minutes each. Slides were cleared in 2 changes of xylene for 20 seconds each, then air dried. Coverslips were mounted using VectaMount (Vector Laboratories, Burlingame, Calif.). Slides were air dried, then evaluated under the microscope. Images (20×) were acquired using an Olympus CX41 microscope equipped with an Olympus DP70 camera and DP Controller software.

Out of the 556 NSCLC tumors screened by immunohistochemistry with the ROS-specific Rmab ROS1 D4D6, 9 ROS1-positive tumors were identified. The breakdown was as follows:

Of the 246 adenocarcinomas, 8 (or 3.3%) were positive for ROS1 kinase.

Of the 20 large cell carcinomas, 1 (or 5.0%) were positive for ROS1 kinase.

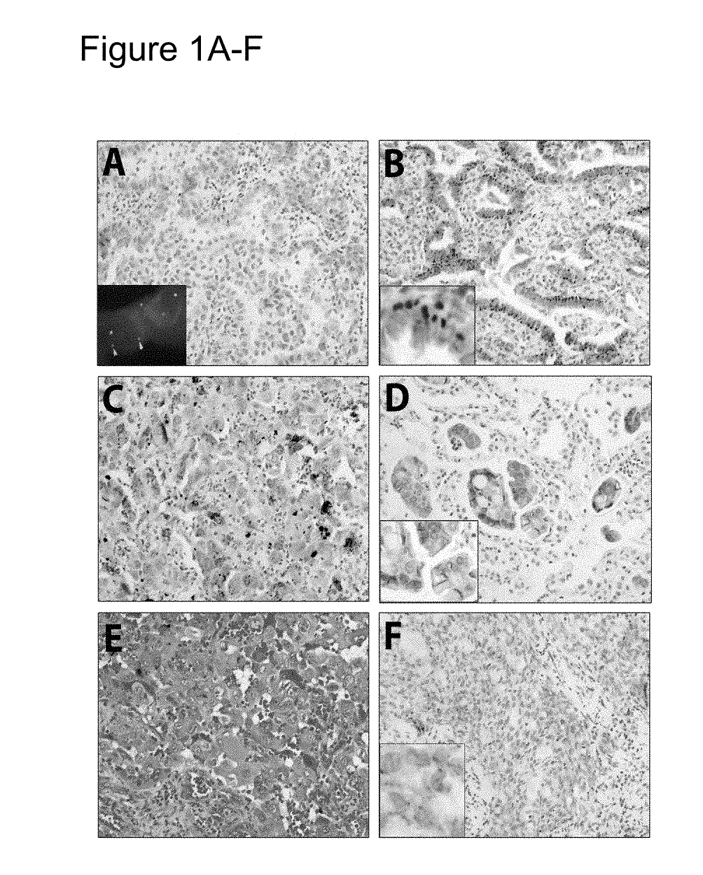

A variety of ROS IHC staining patterns ranging from weak cytoplasmic to strong perinuclear aggregates were observed (see FIGS. 1A-F). In 5/9 (55%) cases ROS localized diffusely in the cytoplasm (FIG. 1A). Strong cytoplasmic staining was observed in 1 large cell carcinoma (FIG. 1C). Two cases had unique phenotypes distinct from each other with one being diffuse cytoplasmic with areas of punctate plasma membrane staining (FIG. 1D) and the other vesicular staining throughout (FIG. 1F). It should also be noted that in rare cases non-neoplastic cells such as macrophages and bronchial epithelial cells stained with ROS D4D6. ROS expression was absent in the surrounding stromal tissue.

### Example 2

### Detection of a ROS Fusion in Human Cancer Samples Using FISH Assay

The presence of either the SLC34A2-ROS fusion protein and/or the CD74-ROS protein (or another ROS fusion protein) in human NSCLC tumor samples was detected using a fluorescence in situ hybridization (FISH) assay, as previously described. See, e.g., Verma et al. HUMAN CHROMOSOMES: A MANUAL OF BASIC TECHNIQUES, Pergamon Press, New York, N.Y. (1988). Over 200 paraffin-embedded human NSCLC tumor samples were examined.

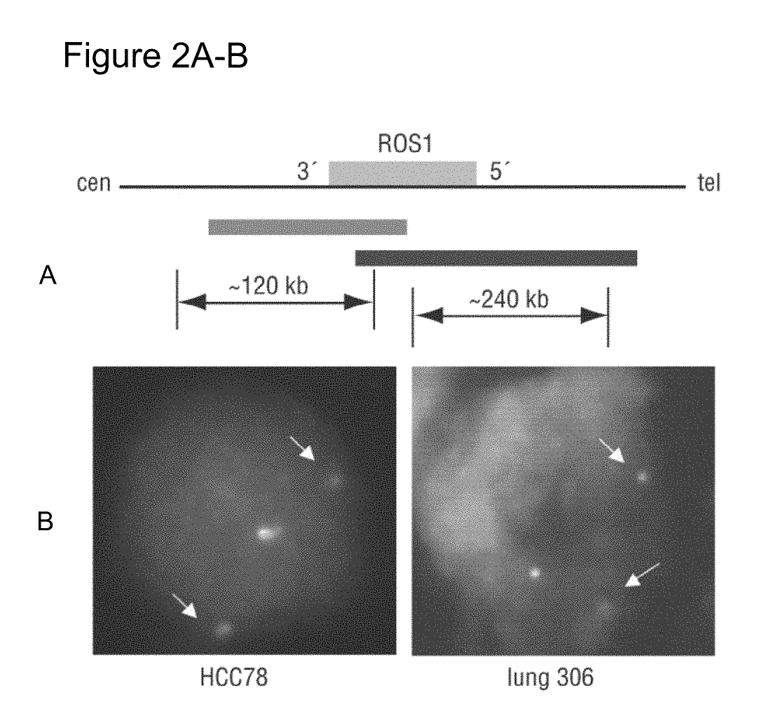

For analyzing rearrangements involving ROS, a dual color break-apart probe was designed. A proximal probe (BAC clone RP1-179P9) and two distal probes (BAC clone RP11-323O17, RP1-94G16) (all of which are commercially available, for example, from Invitrogen Inc., Carlsbad, Calif., as Catalog Nos. RPCI1.C and RPCI11.C) were obtained. The locations at which these probes bind to the ROS gene are shown schematically in FIGS. 2A and 2B. As shown in FIG. 2A, the proximal probe was labeled with Spectrum Orange dUTP, and the distal probes were labeled with Spectrum Green dUTP. Labeling of the probes was done with the Nick Translation DNA Labeling Kit according to manufacturer's instructions (Enzo Life Sciences, Farmingdale, N.Y.). FISH was performed on 4-μm thick FFPE tissue sections according to standard methods. For example, the paraffin embedded tissue sections were re-hydrated and subjected to microwave antigen retrieval in 0.01M Citrate buffer (pH 6.0) for 11 minutes. Sections were digested with Protease (4 mg/ml Pepsin, 2000-3000U/mg) for 25 minutes at 37° C., dehydrated and hybridized with the FISH probe set at 37° C. for 18 hours. After washing, 4′,6-diamidino-2-phenylindole (DAPI; mg/ml) in Vectashield mounting medium (Vector Laboratories, Burlingame, Calif.) was applied for nuclear counterstaining.

FISH-positive cases for ROS were defined as >15% split signals in tumor cells. The Nikon C1 Confocal microscope, 60× objective and trifilter (dapi, TRITC, FITC) was used for scoring each case. For image acquisition the Olympus BX-51 widefield fluorescence microscope with 40× objective and Metamorph software was used to generate tricolor images.

Thus, the ROS rearrangement probe contains two differently labeled probes on opposite sides of the breakpoint of the ROS gene in the wild type (WT) sequence (see FIG. 2A). When hybridized, the native ROS region will appear as an orange/green fusion signal, while rearrangement at this locus (as occurs in the SLC34A2-ROS fusion protein) will result in separate orange and green signals.

As shown in FIG. 2B, a rearranged ROS gene was found in HCC78 (FIG. 2B, left panel) which, as described above, contains a gene rearrangement resulting in the SLC34A2-ROS fusion. In one of the human lung samples, namely lung 306, a similar ROS gene rearrangement was found which may be SLC34A2-ROS or CD74-ROS.

The FISH analysis revealed a low incidence of this ROS mutation in the sample population studied. Of the initial 123 tumors screened, two out of 123 tumors or 1.6% of tumors contained the ROS fusion mutations. However, given the high incidence of NSCLC worldwide (over 151,00 new cases in the U.S. annually, alone), there are expected to be a significant number of patients that harbor this mutant ROS, which patients may benefit from a ROS-inhibiting therapeutic regime.

### Example 3

### Discovery of FIG-ROS Positive NSCLC Tumor

From Example 1, one of the tumor samples, namely Tumor 749, showed ROS1 staining that was localized to vesicular compartments (see FIG. 1F). This staining pattern is distinct from all other ROS1 positive tumors, which pointed to the possibility of a different ROS1 fusion partner.

To determine what the FISH pattern of this Tumor 749 was, a third distal probe RP11-213A17, was obtained from Invitrogen to further investigate whether the ROS mutation in this tumor might be due to a FIG-ROS fusion. Fusions between the FIG gene and the ROS gene have been described in glioblastoma, cholangiocarcinoma, and liver cancer (see Charest et al., Genes Chromosomes Cancer 37: 58-71, 2003; Charest et al., Proc. Natl. Acad. Sci. USA 100: 916-921, 2003; and PCT Publica NO. WO2010/093928), but this fusion has never been described in lung before. Since the fusion between the FIG gene and the ROS gene results not a translocation or inversion but, rather, results from an intrachromosomal deletion on chromosome 6 of 240 kilobases, a new set of FISH probes was designed.

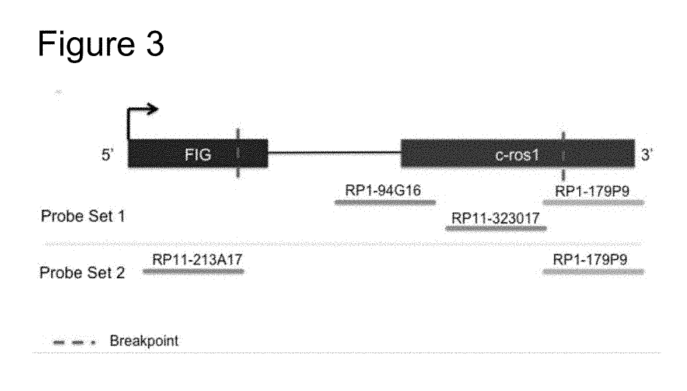

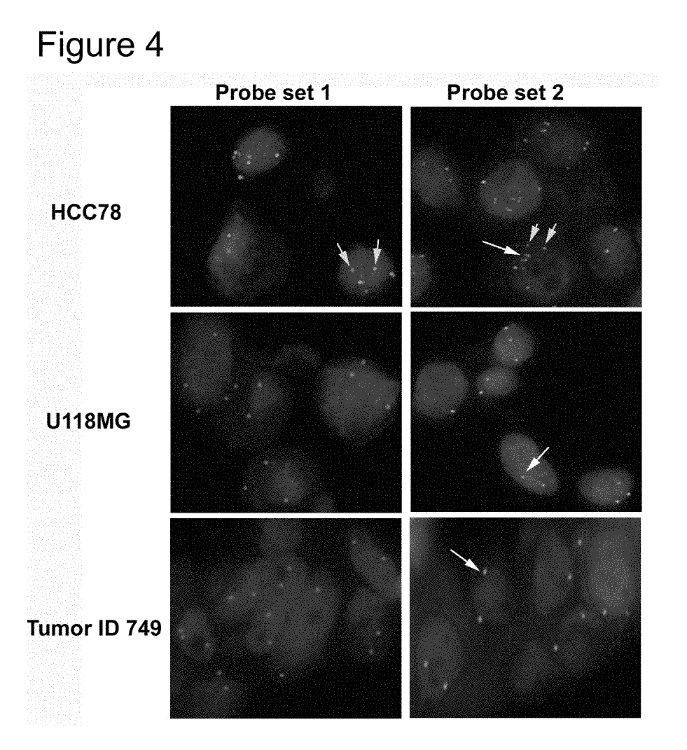

The FISH probes used in the IHC confirmation testing described previously (see Example 2 above) identified those tumors and cells with ROS balanced translocations that could be due to the presence of one of the SLC34A2-ROS fusion protein or the CD74-ROS fusion protein. The FISH pattern in lung 749 suggested that the rearrangement was not one of these two fusions but potentially that of FIG-ROS. To determine if lung ID 749 was indeed FIG-ROS positive, another FISH probe set was designed (FIG. 3). As described above in Example 2, Probe set 1 containing 179P9 and 323O17 BACs flanked either side of the ROS breakpoint in the ROS fusion proteins described herein (e.g., after exon 34, 35, or 36 of ROS) (see FIG. 3 and FIG. 2A). In SLC34A-ROS positive HCC78 cells (see FIG. 2B, left panel and FIG. 4), probe set 1 results in a balanced translocation. In the FIG-ROS positive human U118MG glioblastoma cell line, the 323O17 BAC did not hybridize, since this section of chromosome 6 is deleted, resulting in only orange signals (FIG. 4). Probe set 2 contained 179P9 located on ROS and 213A7 located on the FIG gene, thus U118MG shows both orange and green signals with this probe set (see FIG. 4). HCC78 cells showed 1 chromosome with a balanced translocation (e.g., from a SLC34A2-ROS fusion; see the two yellow arrows in FIG. 4) and the white arrow in FIG. 4 points to a normal chromosome with the green and orange signals close together since the FIG gene and the ROS gene are, in fact, close together on the same chromosome (see FIG. 4). The wild-type chromosome displayed a separated signal due to the distance between the probes. Lung ID 749, when probed with either probe set 1 (FIG. 4) or probe set 2 (see FIG. 4), mimicked that of U118MG cells (FIG. 4). These data were the first to shown the FIG-ROS fusion as an intrachromosomal deletion on chromosome 6 in NSCLC.

### Example 4

### Isolation & Sequencing of the FIG-ROS(S) Fusion Gene from Lung Tumor 749

To isolate and sequence the ROS fusion from tumor 749 (which was a Formalin-Fixed, Paraffin-Embedded Tumor), the following protocol was used.

RT-PCR from FFPE tumor samples: RNA from 3×10 μm sections was extracted following standard protocols (RNeasy FFPE Kit, Qiagen). First strand cDNA was synthesized from 500 ng of total RNA with the use of SuperScript III first strand synthesis system (Invitrogen) with gene specific primers. Then the FIG-ROS fusion cDNA was amplified with the use of PCR primer pairs FIG-F3 and ROS-GSP3.1 for the short isoform and FIG-F7 and ROS-GSP3.2 for the long isoforms. GAPDH primers were purchased from Qiagen (Valencia, Calif.).

Primers
ROS-GSP3.1:
(SEQ ID NO: 15)
CAGCAAGAGACGCAGAGTCAGTTT
ROS-GSP3.2:
(SEQ ID NO: 7)
GCAGCTCAGCCAACTTCTTTTGTCTT
FIG-F3:
(SEQ ID NO: 16)
GCTGTTCTCCAGGCTGAAGTATATGG
FIG-F7:
(SEQ ID NO: 17)
GTAACCCTGGTGCTAGTTGCAAAG

The primers for FIG were selected because based on the FISH patterns observed in tumor 749 and the published information on the FIG-ROS fusion, tumor 749 was expected to be a FIG-ROS fusion.

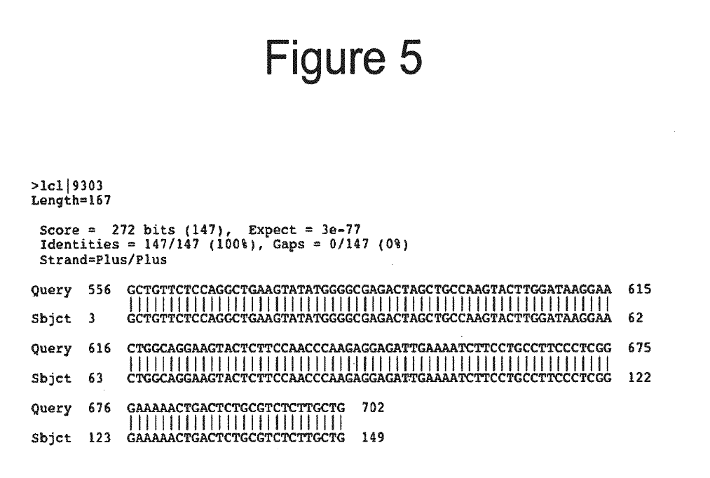

As predicted, the ROS fusion protein in tumor 749 was indeed a FIG-ROS fusion, specifically the FIG-ROS (S) fusion previously described (see PCT Publication No. WO 2010/093928). FIG. 5 shows an alignment of the sequence from the FFPE block from tumor 749 (in the “sbjct” line) with the sequence from the FIG-ROS(S) described in PCT Publication No. WO 2010/093928 (in “query” line). As shown in FIG. 5, the identity was 100% with 0 gaps. Since FIG-ROS(S) contains the entire kinase domain of ROS kinase, this FIG-ROS(S) is expected to retain kinase activity and, thus, is a protein with ROS kinase activity as described herein.

The amino acid sequence of FIG-ROS(S) is set forth in SEQ ID NO: 21 and the nucleotide sequence of FIG-ROS(S) is set forth in SEQ ID NO: 20.

FIG-ROS(L) in liver cancer has also been described (see PCT Publication No. WO 2010/093928). The amino acid and nucleotide sequence of FIG-ROS(L) is set forth in SEQ ID NOs 19 and 18, respectively. In addition, based on analysis of the gene structure of the FIG and the ROS genes, a third FIG-ROS variant (namely FIG-ROS(XL) has been proposed (see PCT Publication No. WO 2010/093928). The amino acid and nucleotide sequence of FIG-ROS(XL) is set forth in SEQ ID NOs 23 and 22, respectively. Given this finding of FIG-ROS(S) in NSCLC, other variants of FIG-ROS fusion protein may also be found in NSCLC.

### Example 5

### Detection of ROS Kinase Expression in a Human Lung Cancer Sample Using PCR Assay

The presence of aberrantly expressed full length ROS protein or a ROS fusion protein (e.g., one of the SLC34A2-ROS fusion proteins, CD74-ROS fusion protein, or one of the FIG-ROS fusion proteins) in a human lung cancer sample may be detected using either genomic or reverse transcriptase (RT) polymerase chain reaction (PCR), previously described. See, e.g., Cools et al., N. Engl. J. Med. 348: 1201-1214 (2003).

Briefly and by way of example, tumor or pleural effusion samples may be obtained from a patient having NSCLC using standard techniques. PCR probes against truncated ROS kinase, SLC34A2-ROS fusion protein, CD74-ROS, or FIG-ROS are constructed. RNeasy Mini Kit (Qiagen) may be used to extract RNA from the tumor or pleural effusion samples. DNA may be extracted with the use of DNeasy Tissue Kit (Qiagen). For RT-PCR, first-strand cDNA is synthesized from, e.g., 2.5 mg of total RNA with the use, for example, of SuperScript™ III first-strand synthesis system (Invitrogen) with oligo (dT)₂₀. Then, the ROS gene or ROS fusion gene (e.g., SLC34A2-ROS, CD74-ROS, or FIG-ROS) is amplified with the use of primer pairs, e.g. SLC34A2-F1 and ROS-P3 (see Example 5 above). For genomic PCR, amplification of the fusion gene may be performed with the use of Platinum Taq DNA polymerase high fidelity (Invitrogen) with primer pairs, e.g. SLC34A2-F1 and ROS-R1, or SLC34A2-F1 and ROS-R2.

Such an analysis will identify a patient having a cancer characterized by expression of the truncated ROS kinase (and/or ROS fusion protein such as FIG-ROS, SLC34A2-ROS, or CD74-ROS), which patient is a candidate for treatment using a ROS-inhibiting therapeutic.

### Example 6

### Sensitivity of ROS Kinase Fusions to TAE-684 and Crizotinib

The small molecule, TAE-684, a 5-chloro-2,4-diaminophenylpyrimidine, inhibits the ALK kinase. The structure of TAE-684 is provided in Galkin, et al., Proc. National Acad. Sci 104(1) 270-275, 2007, incorporated by reference. Another small molecule, namely crizotinib, also inhibits the ALK kinase, as well as the MET kinase. The structure of crizotinib (also called PF-02341066) is provided in Zou H Y et al., Cancer Research 67: 4408-4417, 2007 and U.S. Patent Publication No. 20080300273, incorporated by reference.

Whether TAE-684 and/or crizotinib also inhibits ROS fusion polypeptides was determined.

BaF3 and Karpas 299 cells were obtained from DSMZ (Deutsche Sammlung von Mikroorganismen und Zellkulturen GmbH, Germany). BaF3 cells, which need interleukin-3 to survive, were maintained at 37° C. in RPMI-1640 medium (Invitrogen) with 10% fetal bovine serum (FBS) (Sigma) and 1.0 ng/ml murine IL-3 (R&D Systems). Karpas 299 cells (a lymphoma cell line) were grown in RPMI-1640 with 10% FBS.

BaF3 cells were transduced with retrovirus encoding FIG-ROS(S), FIG-ROS(L), or FLT-3ITD (the Internal tandem duplication mutation in FLT3 causes AML leukemia), and selected for IL3 independent growth. Karpas 299 cells, which express NPM-ALK, was used as a positive control. Retroviruses were generated as previously described (see PCT Publication No. WO 2010/093928, incorporated by reference).

A MTS assay was performed using the CellTiter 96 Aqueous One Solution Reagent (Promega, Catalog No G3582). Briefly, 1×10⁵ cells/well in 24 well plates were grown in 1 mL medium that included 0 nM, 3 nM, 10 nM, 30 nM, 100 nM, 300 nM or 1000 nM TAE-684. After 72 hours, 20 μl of the CellTiter 96 Aqueous One Solution Reagent was added into each well of a 96 well assay plate (flat bottom), and then 100 μl of cells grown with or without treatment. Media-only wells were used as controls. The 96 well plate was incubated for 1-4 hours at 37° C., and then viable cells were counted by reading the absorbance at 490 nm using a 96 well plate reader.

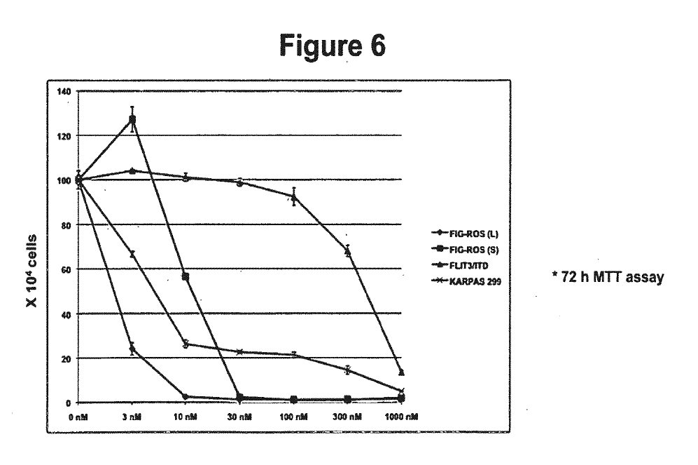

As shown in FIG. 6, the BaF3 cells transduced with retrovirus expressing one of the FIG-ROS polypeptides stopped growing in the presence of TAE-684. FIG-ROS(S) was less susceptible to TAE-684 than FIG-ROS(L). Karpas 299 cells also responded (i.e., stopped growing) in the presence of TAE-684. The BaF3 cells transduced with FLT3/ITD were not susceptible to TAE-684. The IC50 values from two experiments are as follows in Table 4, with data from a final cell line, namely BaF3 cells expressing myc-tagged neomycin, available only in the second experiment.

TABLE 4

| TAE-684 | IC50 | IC50 |
|---|---|---|
| FIG-ROS (L) | 1.78 nM | 2.84 nM |
| FIG-ROS (S) | 10.16 nM | 15.01 nM |
| FLT3/ITD | 419.35 nM | 316.44 nM |
| Neo-Myc | NA | 1641.84 nM |
| Karpas-299 | 4.85 nM | 4.36 nM |

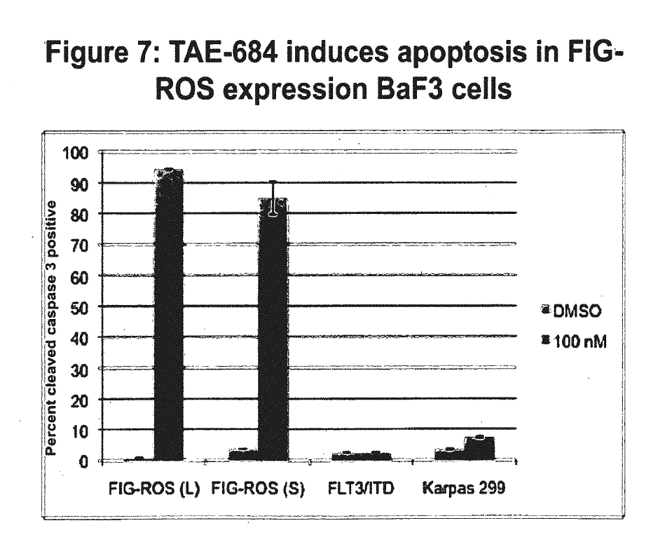

The mechanism of death of the BaF3 and Karpas 299 cells was next assessed by measuring the percentage of cleaved-caspase 3 positive cells by flow cytometry assay using cleaved caspase-3 as a marker for apoptosis. These results were obtained using the protocol publicly available from Cell Signaling Technology, Inc. (Danvers, Mass.). As shown in FIG. 7, the presence of TAE-684 caused the BaF3 cells expressing FIG-ROS(S) or FIG-ROS(L) to die by apoptosis. Karpas 299 cells, which stopped growing in the presence of TAE-684, did not die by apoptosis—they simply underwent cell cycle arrest. Thus, the mechanism by which TAE-684 inhibits FIG-ROS fusion polypeptides is different from the mechanism by which TAE-684 inhibits the ALK kinase.

To further identify the mechanism of action of TAE-684 on the FIG-ROS fusion polypeptides, all four cell lines (i.e., Karpas 299 cells and BaF3 cells transduced with retrovirus encoding FIG-ROS(S), FIG-ROS(L), and FLT-3ITD) were subjected to Western blotting analysis following treatment with 0, 10, 50, or 100 nM TAE-684 for three hours. All antibodies were from Cell Signaling Technology, Inc. (Danvers, Mass.)

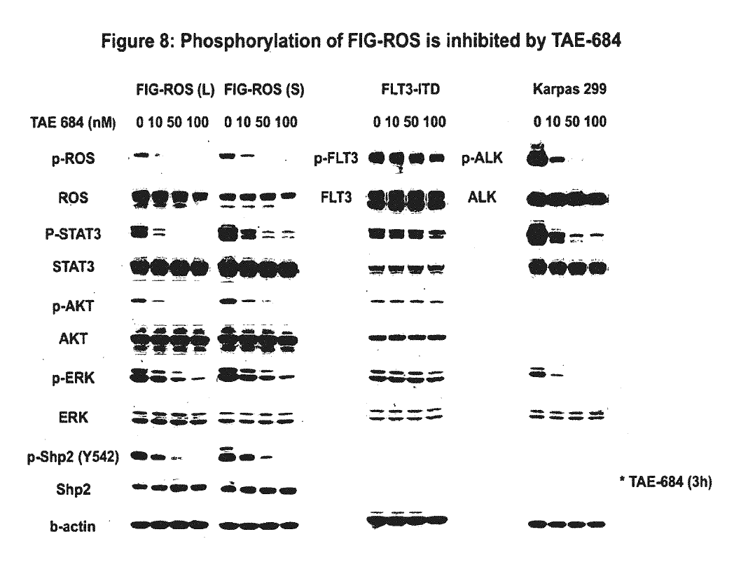

As shown in FIG. 8, phosphorylation of both FIG-ROS(S) and FIG-ROS(L) in FIG-ROS(S) and FIG-ROS(L) expressing BaF3 cells was inhibited by TAE-684. In addition, phosphorylation of STAT3, AKT, and ERK, and Shp2 were inhibited in FIG-ROS(S) and FIG-ROS(L) expressing BaF3 cells. The phosphorylation of STAT3, AKT, and ERK, and Shp2 was not affected in the BaF3 cells transduced with the FLT-3ITD retrovirus. TAE-684 also inhibited ALK and ERK phosphorylation in Karpas 299 cells. Since ROS, ALK, LTK, InsR, and IGFR belong to the same family of tyrosine kinases, they may share similar structure in the kinase domain. Kinase inhibitors or antibodies designed against ALK, LTK, InsR, and IGFIR may have therapeutic effects against ROS kinase.

A parallel set of experiments was next done on the same cells using the same protocols with the addition of another negative control, namely BaF3 cells transduced with the neo-myc tag, to compare two ALK therapeutics, namely TAE-684 and crizotinib.

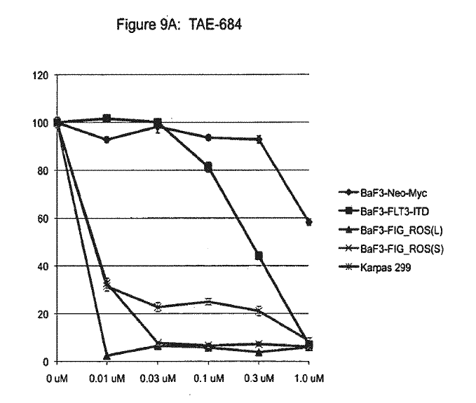

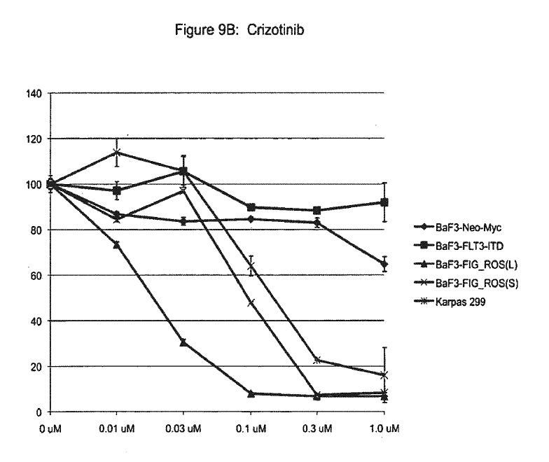

As shown in FIG. 9A (TAE-684) and FIG. 9B (crizotinib), the FIG-ROS fusion protein-containing BaF3 cells were more sensitive to TAE-684 than to crizotinib at the same concentration of each therapeutic. It may be that crizotinib is not as effective as a similar dose of TAE-684, since even the positive control, namely the NPM-ALK fusion protein-expressing Karpas 299 cells, were not sensitive to crizotinib as compared to TAE-684 at the same concentrations. Both of the negative controls (i.e., BaF3 transduced with FLT3-ITD or BaF3 transduced with neo-myc) were less sensitive to crizotinib and to TAE-684 than the FIG-ROS protein-expressing BaF3 cells and the NPM-ALK protein-expressing Karpas 299.

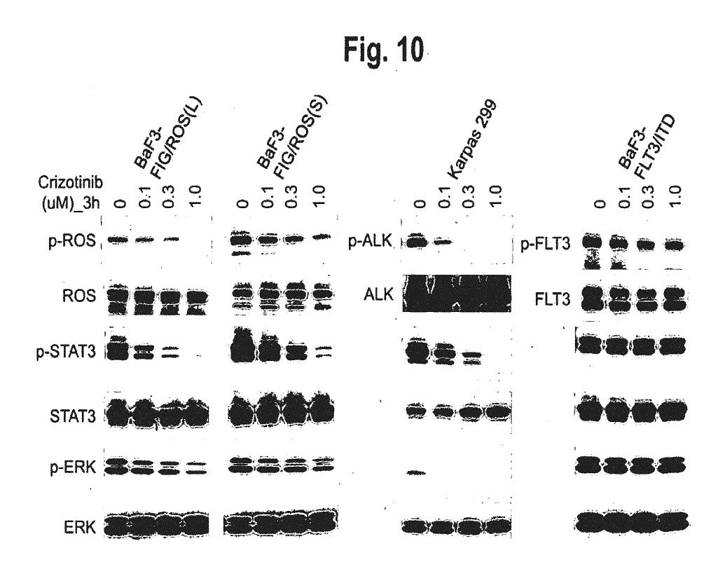

Western blotting analysis following treatment with 0, 0.1, 0.3, or 1.0 uM crizotinib for three hours was next performed using antibodies available from Cell Signaling Technology, Inc. As shown in FIG. 10, phosphorylation of both FIG-ROS(S) and FIG-ROS(L) in FIG-ROS(S) and FIG-ROS(L) expressing BaF3 cells was inhibited by crizotinib. In addition, phosphorylation of STAT3 and ERK, were inhibited by crizotinib in FIG-ROS(S) and FIG-ROS(L) expressing BaF3 cells. The phosphorylation of STAT3 and ERK was not affected in the BaF3 cells transduced with the FLT-3ITD retrovirus following crizotinib treatment. Crizotinib also inhibited ALK, STAT3 and ERK phosphorylation in Karpas 299 cells. Since ROS, ALK, LTK, InsR, and IGFIR belong to the same family of tyrosine kinases, they may share similar structure in the kinase domain. Kinase inhibitors or antibodies designed against ALK, LTK, InsR, and IGFIR may have therapeutic effects against ROS kinase.

### Example 15

### Survey of NSCLC Expressing ALK and/or ROS

In addition to ROS kinase, NSCLC have also been described which contain proteins having ALK activity (see, e.g., U.S. Pat. Nos. 7,700,339; 7,605,131; 7,728,120). Using the IHC methods described above in Example 1, numerous FFPE samples of human NSCLC tumors were screened for specific binding by anti-ROS or anti-ALK antibodies. Such antibodies are commercially available from numerous sources.

The same samples were also screened with FISH for the ROS gene or for the ALK gene using standard methods. For example, a FISH protocol for the ROS gene is described in the Examples above. A FISH protocol for the ALK is described in U.S. Pat. No. 7,700,339, herein incorporated by reference. Likewise, another FISH assay is described in US Patent Publication No. 20110110923, incorporated herein by reference). The results of the screening are shown below in Tables 5 (ROS positive samples) and 6 (ALK positive samples).

TABLE 5
Histopathology of ROS1 positive samples

| Patient | Tumor | | | ROS1 |
|---|---|---|---|---|
| No. | ID | Diagnosis | Histologic pattern (%) | FISH |
| 1 | 147 | Adenocarcinoma | BAC (40), papillary | + |
| | | | (30), Acinar (20), | |
| | | | Solid (10) | |
| 2 | 306 | Adenocarcinoma | Acinar (70), papillary | + |
| | | | (20), and solid (10) | |
| 3 | 570 | Adenocarcinoma | Acinar (90), BAC | + |
| | | | (5), micropapillary (5) | |
| 4 | 400037 | Adenocarcinoma | Acinar | + |
| 5 | 668 | Adenocarcinoma | Solid (80), Acinar | + |
| | | | (10), BAC (10) | |
| 6 | 702 | Adenocarcinoma | Papillary (40), | + |
| | | | Acinar (30), Solid | |
| | | | (30) | |
| 7 | 749 | Adenocarcinoma | Solid (80), Acinar | +, green |
| | | | (20) | deletion |
| 8 | 760 | Adenocarcinoma | Signet cells | + |
| 9 | 575 | Large Cell | | Not |
| | | | | scoreable |

TABLE 6
Histopathology of ALK positive cases.

| Patient | Tumor | | | ALK |
|---|---|---|---|---|
| No. | ID | Diagnosis | Histologic Pattern (%) | FISH |
| 1 | 187 | Adenocarcinoma | Solid | + |
| | | | Focal signet cell ring | |
| | | | features | |
| 2 | 307 | Adenocarcinoma | BAC (30), Acinar (10), | + |
| | | | papillary (10), solid (50) | |
| | | | clear cell and mucinous | |
| | | | features | |
| 3 | 587 | Adenocarcinoma | Acinar (85), solid (10), | Not |
| | | | papillary (5) | score- |
| | | | | able |
| 4 | 618 | Adenocarcinoma | Solid | + |
| 5 | 645 | Adenocarcinoma | Solid (70), BAC (30) | + |
| 6 | 652 | Adenocarcinoma | Papillary (60), | + |
| | | | Micropapillary (40) | |
| 7 | 663 | Adenocarcinoma | Papillary (50) BAC (50) | + |
| 8 | 664 | Adenocarcinoma | Acinar | + |
| 9 | 666 | Adenocarcinoma | Solid (90), Papillary | + |
| | | | (10) | |
| 10 | 670 | Adenocarcinoma | Solid (60), Papillary | + |
| | | | (40) | |
| 11 | 680 | Adenocarcinoma | Solid (70) and acinar | + |
| | | | (30) with signet ring | |
| | | | cell features | |
| 12 | 759 | Adenocarcinoma | Solid with signet ring | + |
| | | | cells | |
| 13 | 580 | Adenocarcinoma | | + |
| | | (uncertain) | | |
| 14 | 70 | Adenocarcinoma | Solid | + |
| 15 | 383 | Adenocarcinoma | BAC (40), papillary | + |
| | | | (30), Acinar (30) | |
| 16 | 395 | Adenocarcinoma | Solid | + |
| 17 | 278 | Squamous; large | | + |
| | | cell carcinoma | | |
| | | (uncertain) | | |
| 18 | 330 | Large cell | | + |
| | | neuroendocrine | | |
| | | carcinoma | | |
| 19 | 503 | Squamous | | + |
| 20 | 615 | Squamous | | + |
| 21 | 644 | Squamous | | + |
| 22 | 691 | Squamous | | + |

Based on this screening of human NSCLC by both IHC and by FISH, it was found that ALK and ROS expression in these tumors is mutually exclusive. In other words, if an NSCLC tumor is driven by ALK, it will not express ROS. Likewise, if an NSCLC tumor is driven by ROS, it will not express ALK. Thus, a therapeutic such as crizotinib or TAE-684 that inhibits both ROS activity and ALK activity will be particularly effective in treating NSCLC.

### EQUIVALENTS

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a patient for cancer, comprising:
detecting the presence in a biological sample from a patient having or suspected of having cancer of a polypeptide selected from the group consisting of a polypeptide having ROS kinase activity and a polypeptide having ALK kinase activity;
and administering a therapeutically effective amount of an ALK- and/or ROS-inhibiting therapeutic to the patient, thereby treating the patient for cancer.

2. The method of claim 1, wherein the detecting step is performed by using a reagent that specifically binds to a polynucleotide encoding either a polypeptide having ROS kinase activity or a polypeptide having ALK kinase activity.

3. The method of claim 1, wherein the detecting step is performed by using a reagent that specifically binds to either a polypeptide having ROS kinase activity or a polypeptide having ALK kinase activity.

4. A method for identifying a patient with cancer or suspected of having cancer as a patient likely to respond to an ALK-inhibiting and/or a ROS-inhibiting therapeutic, comprising:
contacting a biological sample from a patient with a first reagent that specifically binds a polypeptide having ROS kinase activity and a second reagent that specifically binds to a polypeptide having ALK knase activity, and
detecting whether the first reagent or the second reagent specifically binds to the biological sample, wherein detection of binding of either the first reagent or the second reagent to the biological sample identifies the patient as a patient likely to respond to an ALK- and/or ROS-inhibiting therapeutic.

5. A method for identifying a patient with cancer or suspected of having cancer as a patient likely to respond to an ALK- and/or ROS-inhibiting therapeutic, comprising:
contacting a biological sample from a patient with a first reagent that specifically binds a polypeptide having ROS kinase activity or specifically binds to a polynucleotide encoding a polypeptide having ROS kinase activity and a second reagent that specifically binds to a polypeptide having ALK knase activity or specifically binds to a polynucleotide encoding a polypeptide having ALK kinase activity, and
detecting whether the first reagent or the second reagent specifically binds to the biological sample, wherein detection of binding of either the first reagent or the second reagent to the biological sample identifies the patient as a patient likely to respond to an ALK- and/or ROS-inhibiting therapeutic.

6. The method of claim 4 or 5, wherein the first reagent specifically binds to full length ROS kinase protein.

7. The method of claim 4 or 5, wherein the second reagent specifically binds to full length ALK kinase protein.

8. The method of claim 4 or 5, wherein the first reagent specifically binds to the kinase domain of ROS kinase protein.

9. The method of claim 4 or 5 wherein the second reagent specifically binds to the kinase domain of ALK kinase protein.

10. The method of claim 4 or 5 wherein the first reagent is an antibody.

11. The method of claim 4 or 5 wherein the second reagent is an antibody.

12. The method of claim 3, wherein the reagent is an antibody.

13. The method of claim 2, wherein the reagent is a nucleic acid probe.

14. The method of claim 4 or 5, wherein the reagent is a nucleic acid probe.

15. The method of claim 4 or 5, wherein the reagent is a nucleic acid probe.

16. The method of claim 1, 4, or 5, wherein the patient is a human patient.

17. The method of claim 1, 4, or 5, wherein the cancer or suspected cancer is from a human.

18. The method of claim 1, 4, or 5, wherein the ROS-inhibiting therapeutic or the ALK-inhibiting therapeutic is PF-02341066, NVT TAE-684, AP26113, CEP-14083, CEP-14513, CEP11988, CH5424802, WHI-P131 and WHI-P154.

19. The method of claim 1, 4, or 5, wherein the biological sample is from the cancer or suspected cancer of the patient.

20. The method of claim 1, 4, or 5, wherein the cancer is a solid tumor cancer.

21. The method of claim 1, 4, or 5, wherein the cancer is leukemia or lymphoma.

22. The method of claim 1, 4, or 5, wherein the cancer is lung cancer, brain cancer, liver cancer, colon cancer, kidney cancer, breast cancer or ovarian cancer.

23. The method of claim 1, 4, or 5, wherein the biological sample is selected from the group consisting of a tumor biopsy, a bronchoalveolar lavage, a circulating tumor cell, a tumor resection, a fine needle aspirate, a lymph node, a bone marrow sample, and an effusion (e.g., pleural effusion).

24. The method of claim 1, 4, or 5, wherein the reagents are detectably labeled.

25. The method of claim 1, 4, or 5, wherein the polypeptide having ROS kinase activity is a full-length ROS polypeptide or is a ROS fusion polypeptide.

26. The method of claim 1, 4, or 5, wherein the polypeptide having ALK kinase activity is full length ALK polypeptide or is an ALK fusion polypeptide.

27. The method of claim 1, 4, or 5, wherein the method is implemented in a format selected from the group consisting of a flow cytometry assay, an in vitro kinase assay, an immunohistochemistry (IHC) assay, an immunofluorescence (IF) assay, an Enzyme-linked immunosorbent assay (ELISA) assay, and a Western blotting analysis assay.

28. The method of claim 2 or 5, wherein the reagent is detectably labeled.

29. The method of claim 2 or 5, wherein the reagent is a fluorescence in-situ hybridization (FISH) probe and said method is implemented in a FISH assay.

30. The method of claim 2 or 5, wherein the reagent is a polymerase chain reaction (PCR) probe and said method is implemented in a PCR assay.

31. A method for inhibiting the progression of a mammalian cancer or suspected mammalian cancer that expresses either a first polypeptide having ROS kinase activity or a second polypeptide having ALK kinase activity, said method comprising the step of inhibiting the expression and/or activity of said first or said second polypeptide in said mammalian cancer or suspected mammalian cancer.

32. A method for inhibiting the progression of a mammalian cancer or suspected mammalian cancer comprising a first polynucleotide encoding a polypeptide having ROS kinase activity or a second polynucleotide encoding a polypeptide having ALK kinase activity, said method comprising the step of inhibiting the expression of said first or said second polynucleotide in said mammalian cancer or suspected mammalian cancer.

33. The method of claim 31 or 32, wherein the mammalian cancer or suspected mammalian cancer is from a human.
